(19) United States
(12) Patent Application Publication
(10) Pub. No.: US 2020/0020420 A1
(43) Pub. Date: Jan. 16, 2020

(54) Method for Establishing Machine Learning Model for Predicting Toxicity of siRNA to Certain Type of Cells and Application Thereof

(71) Applicant: Hangzhou Converd Co., Ltd., Hangzhou (CN)

(72) Inventors: Jinlu Cai, Hangzhou (CN); Nan Zhong, Hangzhou (CN); Qingyong Zhang, Hangzhou (CN); Ying Jin, Hangzhou (CN); Xiuqin Zhang, Hangzhou (CN)

(21) Appl. No.: 16/465,303

(22) PCT Filed: Dec. 7, 2017
(86) PCT No.: PCT/CN2017/114938

§ 371 Date: May 30, 2019

(30) Foreign Application Priority Data
Dec. 8, 2016 (CN) 201611121663.4

(51) Int. Cl. G16B 40/20; G16B 30/10

(52) U.S. Cl. CPC G16B 40/20; G16B 30/10

(57) ABSTRACT

Provided is a method of establishing a machine learning model for predicting toxicity of siRNA to certain type of cells and application thereof. The method includes A) providing n siRNAs of 19-29 bp, wherein n≥2; B) obtaining input and output values for establishing the model from each siRNA, the input values being obtained by i) aligning each siRNA with genomic mRNAs and selecting complementary off-target genes having no more than 7 mismatched bases; ii) obtaining off-target weights according to mismatched bases' characteristic and mRNA's secondary structure in complementary region; iii) obtaining omic weights of the off-target genes using databases; iv) calculating omic eigenvalues as the input values, based on omic and off-target weights of all the off-target genes; the output values being obtained by conducting experiments with the siRNAs to obtain cell survival indexes; and C) calculating the input and output values of the n siRNAs through machine learning algorithm.

### FIELD OF THE INVENTION

The invention belongs to the field of biotechnology, and particularly relates to a method for establishing a machine learning model for predicting toxicity of siRNA to a certain type of cells and its application, a computer readable medium, and an apparatus/method using this model.

### BACKGROUND OF THE INVENTION

RNA inference (RNAi) technology is a breakthrough in the field of biomedicine in the past decade. RNAi refers to a phenomenon of gene silencing induced by double-stranded RNA in molecular biology. When a double-stranded RNA homologous to the endogenous mRNA coding region is introduced into a cell, the mRNA is degraded or the translation is inhibited to cause the silencing of the gene expression. RNAi technology can shut down the expression of specific genes and it is a rapid and effective tool for inhibiting gene expression. It has been widely used in the field of gene therapy for viral related diseases (mainly AIDS and hepatitis) and malignant tumors. On the one hand, RNAi is the touchstone for testing gene function. RNAi technology can greatly shorten the time of human cognition of gene function. On the other hand, RNAi technology can be used to develop new drugs that inhibit pathogenic genes, namely small interfering nucleic acids (small inference RNA, siRNA) drugs. RNAi can effectively silence the expression of the target gene and reduce the level of related proteins to amplify the inhibitory effect, which is more thorough than the effect of the inhibition of protein activity by traditional small-molecule or antibody drugs.

The core mechanism of siRNA action is the principle of nucleotide complementary pairing, so the off-target effect is inevitably generated. There occurs non-specificity during the action of siRNA, which may interact with other non-target genes rather than specifically block the expression of the target gene, thereby producing the unexpected side effects. Currently, the siRNA is designed first, then a simple homology alignment is performed to avoid the serious off-target effect of the designed siRNA. For example, when siRNA is used as a human anti-viral drug candidate, if the sequence of the candidate siRNA and the sequence of the human gene substantially match, with only 1-2 base mismatches, this candidate siRNA is no longer considered. However, in fact, when the sequence of the candidate siRNA and the sequence of the human gene have 3 or more base mismatches, the siRNA may still have a certain interference effect on the corresponding human gene, and the synthesis of the corresponding protein may be reduced/inhibited, leading to the production of cytotoxicity. At present, in practice, the cytotoxicity of siRNA is often screened in vitro by a large number of biological experiments. In the development of emergency drugs for viral infectious diseases, it is impossible to solve the problem of quickly providing safe and effective drugs.

### SUMMARY OF THE INVENTION

In order to solve the above-mentioned problems in the prior art, the present invention provides a method of establishing a machine learning model for predicting the toxicity of siRNA to a certain type of cells and its application, a computer readable medium, and an apparatus/method using this model.

In particular, the present invention provides:

(1) A method of establishing a machine learning model for predicting toxicity of an siRNA to a certain type of cells, comprising the following steps:

A) providing n siRNAs, wherein n≥2, and wherein the siRNAs are 19-29 bp in length;

B) separately obtaining an input value and an output value for establishing a machine learning model from each of the siRNAs;

wherein, the input value of any one of the n siRNAs is obtained as follows:
i) aligning a sequence of the siRNA with sequences of genomic mRNAs, respectively, and selecting one or more off-target genes located in the genomic mRNAs, which are complementary to the siRNA and the number of mismatched bases therebetween is less than or equal to 7;
ii) obtaining an off-target weight of each of the selected off-target genes regarding each complementary region of the off-target gene's mRNA to the siRNA sequence, independently, according to characteristic of the mismatched bases and secondary structure characteristic of the off-target gene's mRNA sequence;
iii) independently of ii) and unsequentially with ii), annotating each of the selected off-target genes using bioinformatics databases, and therefore obtaining omic weights of the off-target gene, including at least one selected from the group consisting of: protein interaction weight, signal pathway weight and core gene weight of the off-target gene; and
iv) calculating each omic eigenvalue based on the respective omic weights and the off-target weights of all the selected off-target genes, and using each of the eigenvalues as the input value;

and wherein, the output value of the siRNA is obtained as follows:
using the siRNA to conduct experiments in a certain type of cells to obtain a cell survival index in the presence of the siRNA, and using the cell survival index as the output value; and

C) establishing the machine learning model by calculating all the input values and the output values of the n siRNAs through a machine learning algorithm.

(2) The method according to item (1), wherein the characteristic of the mismatched bases comprises the number of the mismatched bases, and optionally, the position of the mismatched bases.

(3) The method according to item (1) or (2), wherein the secondary structural characteristic of the off-target gene's mRNA sequence is a probability of the mRNA itself not forming a secondary structure in the complementary region.

(4) The method according to item (3), wherein for each of the selected off-target genes, an interference rate of the siRNA on the expression level of the off-target gene's mRNA is calculated according to characteristic of the mismatched bases, and then, a product of the interference rate and the probability of not forming the secondary structure is calculated to obtain the off-target weight of the off-target gene.

(5) The method according to item (3), wherein the probability of the mRNA of each off-target gene not forming a secondary structure is predicted using a software selected from the group consisting of: RNAPLFOLD, mfold or RNAstructure.

(6) The method according to item (1), wherein the omic eigenvalues include at least one selected from the group consisting of: a proteomic eigenvalue, a signal pathwayomic eigenvalue, and a core genomic eigenvalue; and wherein the proteomic eigenvalue, the signal pathwayomic eigenvalue and the core genomic eigenvalue are calculated according to the following a) to c), respectively:

a) calculating a product a′ of the off-target weight of each of the selected off-target genes and its protein interaction weight, and then calculating a sum of all the products a′ obtained for each of the selected off-target genes to generate a proteomic eigenvalue;

b) calculating a product b′ of the off-target weight of each of the selected off-target genes and its signal pathway weight, and then calculating a sum of all the products b′ obtained for each of the selected off-target genes to generate a signal pathwayomic eigenvalue;

c) calculating a product c′ of the off-target weight of each of the selected off-target genes and its core gene weight, and then calculating a sum of all the products c′ obtained for each of the selected off-target genes to generate a core genomic eigenvalue.

(7) The method according to item (1), wherein all the input values are normalized prior to establishing the machine learning model.

(8) The method according to item (1), wherein the machine learning algorithm comprises: a support vector machine, an artificial neural network, a decision tree, or a regression model.

(9) The method according to item (1), wherein in the step i), the selected off-target gene does not comprise such an off-target gene that a complementary region of its mRNA to the siRNA sequence is located only in its 5′ UTR.

(10) The method according to item (1), wherein in the step i), the selected off-target gene does not include a gene which is not expressed in the certain type of cells in a normal state.

(11) Use of the method according to any one of items (1) to (10) for predicting toxicity of an siRNA to a certain type of cells.

(12) A computer readable medium, wherein the computer readable medium can be used to establish the machine learning model on the basis of the method according to any one of items (1) to (10), and the computer readable medium comprises the following modules:

a sequence alignment module for performing the step i) in the method according to any one of items (1) to (10);

an off-target weight calculation module for performing the step ii) in the method according to any one of items (1) to (10);

an omic annotation module for performing the step iii) in the method according to any one of items (1) to (10);

an omic eigenvalue calculation module for performing the step iv) in the method according to any one of items (1) to (10); and

a machine learning algorithm calculation module for performing the step C) in the method according to any one of items (1) to (10).

(13) A device for predicting toxicity of an siRNA to a certain type of cells, comprising:

1) an input unit for inputting a sequence of the siRNA to be tested;

2) a storage unit for storing a machine learning model established for a certain type of cells using the method according to any one of items (1) to (10);

3) an execution unit for executing the machine learning model on the sequence of the siRNA; and

4) an output unit for displaying a predicted result of the toxicity of the siRNA to the certain type of cells.

(14) A method of predicting toxicity of an siRNA to a certain type of cells, comprising:

providing a sequence of the siRNA to be tested;

inputting the sequence of the siRNA to the device according to item (13), and allowing the device to execute the machine learning model established for the certain type of cells using the method according to any one of items (1) to (10), thereby obtaining result of the prediction of the toxicity of the siRNA to the certain type of cells.

Compared with the current techniques, the invention has the following advantages and positive effects: based on big data in bioinformatics and using a bioinformatics analysis method, the invention establishes a machine learning model for predicting the toxicity of siRNA to a certain type of cells, which comprehensively determines the off-target genes of the siRNA to be tested and gives the corresponding weight coefficient. By combining large data such as proteomic data, pathwayomic data and core genomic data, the model can be used to quickly predict the cytotoxicity caused by the off-target effect of the siRNA to be tested, especially in the case of emergency, and therefore, can effectively assist the design of siRNA and shorten the screening time, improve screening efficiency and facilitate the drug development in an emergency.

### BRIEF DESCRIPTION OF THE DRAWINGS

FIG. 1 shows the result of screening for effective siRNA sequences for the MGMT gene, wherein the horizontal axis represents different siRNA groups; and the vertical axis represents the ratio of the mRNA expression level of MGMT of each group to the blank control group.

FIG. 2 shows the result of screening for interference concentrations of effective siRNA sequences for the MGMT genes, wherein the horizontal axis represents the different transfection concentrations of siRNA4; and the vertical axis represents the ratio of the mRNA expression level of MGMT at each transfection concentration to the blank control group.

FIG. 3 shows the relative expression levels of MGMT mRNA in the presence of different mismatched siRNAs, wherein the horizontal axis represents different siRNA groups, and the vertical axis represents the ratio of the mRNA expression level of MGMT of each group to the blank control group.

FIG. 4 is a graph showing the relationship between the interference rate of siRNAs mismatched at the 3′ end of the sense strand and the number of mismatched bases, wherein the horizontal axis represents the number of mismatched bases of the siRNA with mismatches located at the 3′ end of the sense strand, and the vertical axis represents the interference rate of the corresponding siRNA. The solid line connecting the dots represents the actual curve, and the broken line represents the fitting result.

FIG. 5 is a graph showing the relationship between the interference rate of siRNAs mismatched at the 5′ end of the sense strand and the number of mismatched bases, wherein the horizontal axis represents the number of mismatched bases of the siRNA with mismatches located at the 5′ end of the sense strand, and the vertical axis represents the interference rate of the corresponding siRNA. The solid line connecting the dots represents the actual curve, and the broken line represents the fitting result.

FIG. 6 is a schematic view showing a method of calculating proteomic eigenvalues.

FIG. 7 shows the results of cell survival index of A549 cells in the presence of different mismatched siRNAs, wherein the horizontal axis represents different siRNA groups, and the vertical axis represents the cell survival index of each group.

FIG. 8 shows a flow diagram of one embodiment of the process of the invention.

FIG. 9 shows a schematic diagram of one embodiment of a computer readable medium of the present invention.

FIG. 10 is a schematic diagram showing a node connection using 10-fold cross-validation when a machine learning model is established by a machine learning algorithm PNN in an embodiment of the present invention.

FIG. 11 is a schematic diagram showing a node connection using 10-fold cross-validation when a machine learning model is established by a machine learning algorithm SVM in an embodiment of the present invention.

### DETAILED DESCRIPTION OF THE INVENTION

The invention is further described by the following description of the embodiments and with reference to the accompanied drawings, but it is not intended to limit the invention, and those skilled in the art can make various modifications or improvements according to the spirit of the invention. The modifications and improvements are within the scope of the invention, without departing from the spirit of the invention.

siRNA drugs have advantages over other traditional drugs in responding to new viral disease outbreaks. After preliminary acquisition of the sequence of the burst virus, the design, preliminary screening and validation of the siRNA drug for virus inhibition can be completed in a relatively short period of time. However, the siRNA thus obtained usually has an off-target effect and causes cytotoxicity. In an emergent situation, there is an urgent need for a method that can shorten the screening time, improve the screening efficiency, and facilitate the drug development in an emergency to predict the cytotoxicity of siRNA, thereby effectively assisting the design of siRNA.

As used herein, the term “sudden virus” or “burst virus” includes: respiratory virus, Ebola virus, Zika virus, and so on.

As used herein, the term “respiratory virus” is known in the art and refers to a large class of viruses that can invade the respiratory tract causing localized lesions in the respiratory tract, or only invade the respiratory tract while primarily causing lesions in the tissues outside the respiratory tract. Respiratory viruses include influenza viruses in the Orthomyxoviridae family, parainfluenza virus in the Paramyxoviridae family, respiratory syncytial virus, measles virus, mumps virus, and other viruses such as the gland virus, rubella virus, rhinovirus, coronavirus and reovirus. According to statistics, more than 90% of acute respiratory infections are caused by viruses.

As used herein, the term “influenza virus” is known in the art and has three types A, B and C, which will cause influenza (abbreviated as “flu”) in humans and animals (e.g., pigs, horses, marine mammals and poultry, etc.). Influenza A virus is the most important cause of human influenza epidemics, and it is the most frequent and important epidemic pathogens. In taxonomy, influenza viruses belong to the family of Orthomyxoviridae, which will cause acute upper respiratory tract infections and rapidly spread by air, and therefore there are often periodic pandemics around the world. Influenza viruses can cause more serious symptoms, such as pneumonia or cardiopulmonary failure, in elderly or children with weak immunity and in some patients with immune disorders.

Respiratory viruses also include coronaviruses, and a previously unknown coronavirus has caused a global SARS disaster. SARS was launched in 2002 in Guangdong, China, and spreaded to Southeast Asia and even the whole world. Till the mid-2003, this global epidemic was gradually eliminated. Research reports indicate that SARS Coronavirus (SARS-CoV) is the causative agent of severe acute respiratory syndrome (SARS).

As used herein, the term “Ebolavirus” (EBOV) is known in the art and belongs to the family Filofiridae. The virion is filamentous or rod-shaped, having a diameter of about 100 nm and a length of 300 to 1500 nm. The virus particles have a helical nucleocapsid with an outer envelope. Its genome is a single-stranded negative-strand RNA with a total length of about 19 kb, which encodes a total of seven proteins. At present, Ebola virus can be divided into five subtypes: Zaire Ebolavirus (ZE-BOV), Cote d'lvoire Ebolavirus (CE-BOV), Sudan Ebolavirus (SEBOV), Lai Reston Ebolavirus (REBOV) and Bundibugyo Ebolavirus (BEBOV). Ebola hemorrhagic fever (EHF) is an acute hemorrhagic infection caused by the Ebola virus. It first occurred in Zaire (now the Democratic Republic of the Congo) in the Ebola River Basin in 1976. It causes symptoms of systemic bleeding in infected people, so it is named Ebola hemorrhagic fever. Since the outbreak in Zaire (now the Democratic Republic of the Congo) and the Sudan in 1976, a local epidemic has taken place in central Africa, mainly in countries such as Uganda, Congo, Gabon, Sudan, Cote d'lvoire, Liberia, South Africa, etc. It is super contagious, and the mortality rate is as high as 50% to 88%. People are mainly infected by contact with the body fluid, excretions, secretions, etc. of the patients or infected animals. The main clinical manifestations are fever, hemorrhage and multiple organ damage.

As used herein, the term “off-target effect” is known in the art and means that there is non-specific binding during siRNA action, possibly with other genes than the target genes, thus non-specifically blocking gene expression and producing unexpected effects. The off-target effects associated with siRNA fall into three broad categories: microRNA (miRNA)-like off-target effects, immune stimulation, and saturation of RNAi elements.

An object of the present invention is to provide a method of establishing a machine learning model for predicting the toxicity of an siRNA to a certain type of cells. Another object of this invention is to provide use of the method for predicting the toxicity of an siRNA to such cells. The third object of the present invention is to provide a computer readable medium. The fourth object of the present invention is to provide a device for predicting the toxicity of an siRNA to a certain type of cells. The fifth object of the invention is to provide a method of predicting the toxicity of an siRNA to a certain type of cells.

I. Method of Establishing a Machine Learning Model for Predicting Toxicity of an siRNA to a Certain Type of Cells

The first aspect of the invention provides a method of establishing a machine learning model for predicting the toxicity of an siRNA to a certain type of cells, comprising the steps of:

A) providing n siRNAs, wherein n≥2, and wherein the siRNAs are 19-29 bp in length;

B) separately obtaining an input value and an output value for establishing a machine learning model from each of the siRNAs;

wherein, the input value of any one of the n siRNAs is obtained as follows:
i) aligning a sequence of the siRNA with sequences of genomic mRNAs, respectively, and selecting one or more off-target genes located in the genomic mRNAs, which are complementary to the siRNA and the number of mismatched bases therebetween is less than or equal to 7;
ii) obtaining an off-target weight of each of the selected off-target genes regarding each complementary region of the off-target gene's mRNA to the siRNA sequence, independently, according to characteristic of the mismatched bases and secondary structure characteristic of the off-target gene's mRNA sequence;
iii) independently of ii) and unsequentially with ii), annotating each of the selected off-target genes using bioinformatics databases, and therefore obtaining omic weights of the off-target gene, including at least one selected from the group consisting of: protein interaction weight, signal pathway weight and core gene weight of the off-target gene; and
iv) calculating each omic eigenvalue based on the respective omic weights and the off-target weights of all the selected off-target genes, and using each of the eigenvalues as the input value;

and wherein, the output value of the siRNA is obtained as follows:
using the siRNA to conduct experiments in a certain type of cells to obtain a cell survival index in the presence of the siRNA, and using the cell survival index as the output value; and

C) establishing the machine learning model by calculating all the input values and the output values of the n siRNAs through a machine learning algorithm.

The method of establishing a machine learning model of the present invention utilizes bioinformatics in combination with biological experimental data and is calculated by a machine learning algorithm.

As used herein, the term “bioinformatics” is known in the art and refers to the science of storing, retrieving and analyzing biological information using a computer as a tool in life science research. In general, bioinformatics combines molecular biology with information technology, especially Internet technology. Research materials and results of bioinformatics include a wide variety of biological data, with research tools including computers and by research methods including searching (collecting and screening), processing (editing, organizing, managing, and displaying) and using (calculation and simulation) of biological data.

As used herein, the term “machine learning” is known in the art, which is a multi-disciplinary subject involving multiple principles such as probability theory, statistics, approximation theory, convex analysis, computational complexity theory and so on. Machine learning theory is primarily about designing and analyzing algorithms that allow computers to automatically “learn”. The machine learning algorithm belongs to the artificial intelligence algorithm. It is a kind of algorithm that automatically analyzes and obtains the law from the data and predicts the unknown data by using the law. Because learning algorithms involve a large number of statistical theories, machine learning is particularly closely related to inferential statistics, and also known as statistical learning theory. Machine learning can be divided into the following categories: supervised learning, unsupervised learning, semi-supervised learning, and enhanced learning, etc. Supervised learning learns a function from a given set of training data, and when new data arrive, it can predict the outcome based on this function. The training set requirements for supervised learning include input and output, or in other words, characteristics and goals. The goal of the training set is marked by people. Common supervised learning algorithms include regression analysis and statistical classification. Unsupervised learning has no artificially labeled results compared to supervised learning. Common unsupervised learning algorithms have clusters. Semi-supervised learning is between supervised learning and unsupervised learning. Enhanced learning is a process of learning what action to be made through observation. Each action has an impact on the environment, and the learning object makes a judgment based on feedback from the observed surrounding environment.

In one embodiment of the invention, the machine learning algorithm is preferably a supervised learning algorithm.

The machine learning model of the present invention is a machine learning model for predicting the toxicity of siRNA to a certain type of cells.

The cells in the term “a certain type of cells” as used herein in reference to predicting cytotoxicity may be human cells or other mammalian cells. When the cells are human cells, the genomic mRNA is human genomic mRNA. When the cells are other mammalian cells, the genomic mRNA is the genomic mRNA of the specific mammal. In addition, the term “a certain type of cells” refers to one or more types of cells that are functionally identical or related. For example, “a certain type of cells” may be such cells that the virus can contact or infect, such as respiratory epithelial cells, gastrointestinal epithelial cells, skin cells, liver cells, nerve cells, lymphocytes, ocular cells, urethral cells, reproductive tract cells, and the like. When the term “a certain type of cells” refers to a plurality of types of cells, a machine learning model for predicting the toxicity of siRNA to such cells can be established separately for each type of the cells.

As used herein, the term “siRNA (small interfering nucleic acid, also abbreviated as small nucleic acid)” is known in the art and refers to a double-stranded short nucleic acid with a specific gene code, which may be 19-29 bp (base pair) in length. (See the literature: “McIntyre G J, Yu Y H, Lomas M, Fanning G C. The effects of stem length and core placement on shRNA activity. BMC Mol Biol. 2011 Aug. 8; 12:34.”) The strand of the siRNA with the same sequence as the targeting sequence of messenger RNA (mRNA) is called the sense strand, and the other complementary strand is the antisense strand. The siRNA includes a 5′-phosphate terminus, a 19 nt double-stranded region, a 3′-hydroxy terminus, and two unpaired 3′-terminal nucleotide knobs, which can direct cleavage of mRNA. In general, a gene usually contains thousands of bps, and siRNA is a specific sequence of 21 to 23 bp in length. siRNA can be cloned into an siRNA expression vector, which functions to bind to the messenger ribonucleic acid (mRNA) of a specific target gene in a mammalian cell such that the mRNA is degraded and lose the target gene expression to become “silent”, that is, “close” the function of the gene. The mechanism by which the siRNA degrades mRNA to block the synthesis of a specific protein is called nucleic acid interference (RNAi).

As used herein, the term “RNA interference (RNAi)” is known in the art and refers to the phenomenon of efficient and specific degradation of mRNA induced by homologous double-stranded RNA (dsRNA), which is highly conserved during evolution. Once discovered, RNAi quickly became one of the most active and hot topics in the field of biological research. “Science” listed it as one of the top ten scientific achievements in 2001, and in 2002 further ranked it as the first of the top ten technologies. “Nature” also named siRNA one of the most important scientific discoveries of 2002. Two American scientists, Farr and Melo, who discovered the RNAi mechanism in 2006, won the Nobel Prize in Medicine. RNAi technology can specifically eliminate or turn off the expression of specific genes. It is a rapid, effective and specific tool for inhibiting gene expression. It has been widely used to explore gene function, viral diseases (mainly AIDS and hepatitis) and malignant tumors in the field of gene therapy. On one hand, RNAi is the touchstone for testing gene function. RNAi technology can greatly shorten the time for understanding of human gene functions. On the other hand, RNAi technology can be used to obtain novel gene drugs that inactivate disease-causing genes, i.e., siRNA drug.

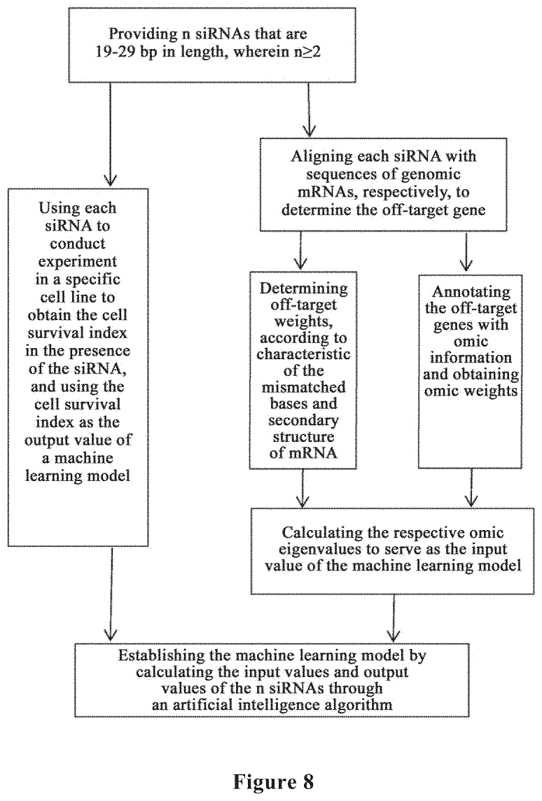

For example, FIG. 8 shows a flow diagram of one embodiment of the method of the present invention. In the method of the invention, n siRNAs are first provided and each siRNA comprises a sense strand sequence and an antisense strand sequence in a pair. The value of n is greater than or equal to 2, for example, greater than or equal to 10, greater than or equal to 15, greater than or equal to 20, greater than or equal to 100, and the like. Those skilled in the art can select suitable value of n, based on actual conditions (e.g., balancing between the demand for model accuracy or other requirements and the demand for time and economic cost control or other requirements).

The n siRNAs may be specifically designed to carry out the method of the invention to establish a machine learning model for predicting the toxicity of siRNA to a certain type of cells, such as those shown in Tables 1 and 2 of Experimental Example 1 of the present specification. The n siRNAs may also be anti-viral candidate siRNA drugs designed for a certain virus, which may be a sudden virus. For example, the n siRNAs can be designed for a specific virus in respiratory viruses or designed for a particular virus in the Ebola viruses.

The method of the present invention further comprises obtaining the input values for establishing the machine learning model by using bioinformatics for each siRNA, and obtaining the output values for establishing the machine learning model by using biological experiments, independently of and unsequentially with the process of obtaining the input value.

In the process of obtaining the input values for each siRNA for establishing the machine learning model according to the method of the invention, in order to initially determine the off-target genes of each siRNA, comprehensive alignment of the sequence of the siRNA to the sequences of the genomic mRNAs is performed, with the number of mismatched bases therebetween being set to be less than or equal to 7, thereby comprehensively select a series of off-target genes.

The genomic mRNA can be human genomic mRNA or other mammalian genomic mRNA. Other mammals include, but are not limited to, for example, chimpanzees, gorillas, bonobos, guinea pigs, pikas, rabbits, squirrels, dogs, cats, mice, rats, and the like.

As used herein, the term “human genome” is known in the art and refers to the genome of human (Homo sapiens), consisting of 23 pairs of chromosomes, containing approximately 3.16 billion DNA base pairs. Some of the base pairs make up about 20,000 to 25,000 genes. All human genome sequencing work was completed in 2006 and the human genome sequence is publicly available.

The siRNA is complementary to mRNA to a different extent and the secondary structure of the mRNA in the complementary region varies, leading to different off-target effects. According to the present invention, the off-target weight of the selected off-target gene regarding each complementary region of the off-target gene's mRNA to the siRNA sequence is determined by the characteristic of the mismatched bases and the secondary structural characteristic of the off-target gene's mRNA sequence.

In addition, the process from genetic influence to cytotoxicity is a complex biological subject, like a black box. The off-target effect of siRNA is mainly embodied in the degradation of mRNA or the inhibition of further translation of mRNA into protein, so the off-target effect at the protein level is the most direct. Proteins are not isolated, and in various signaling pathways in the cells, upstream proteins tend to regulate (including activation or inhibition) the activity of downstream proteins, mainly by adding or removing phosphate groups and changing the stereology of downstream proteins. In addition, among all genes in the human genome, some genes are essential for human living, called core genes, and more than 1,500 core genes are currently known. In order to more scientifically and accurately predict the toxicity of siRNA to a certain type of cells by a machine learning model, the method of the present invention integrates information from big data such as proteomic, signal pathwayomic and/or core geneomic data, followed by annotating the selected off-target genes with these omic information to get the omic weights thereof and calculating each omic eigenvalue based on the respective omic weights and the off-target weights of all the selected off-target genes.

In the process of obtaining the output value for each siRNA for establishing a machine learning model according to the method of the present invention, the type of cells is subjected to an experiment using the siRNA to obtain a cell survival index in the presence of the siRNA, and the cell survival index is used as the output value. The term “cell survival index” as used herein refers to the state of survival of a cell, expressed as the ratio of the OD450 value of a cell in the presence of a given siRNA to the OD450 value of that cell under normal conditions.

Through the above design and concept, the method of the present invention for establishing a machine learning model for predicting the toxicity of siRNA to a certain type of cells becomes more scientific, rigorous, and accurate.

In the method, the length of the siRNA is further preferably from 19 to 25 bp, more preferably from 19 to 21 bp, and still more preferably 21 bp.

The alignment can be performed using alignment software selected from BLAST, BLAT or Wise2DBA. When using the software, one can use the default parameters as needed and adjust some of them to get a comprehensive comparison. Taking BLAST as an example (for description of the software, see the literature: “Camacho C, Coulouris G, Avagyan V, Ma N, Papadopoulos J, Bealer K, Madden TL. BLAST+: architecture and applications. BMC Bioinformatics. 2008, 10: 421.”, the entire content of which is incorporated herein by reference), in one embodiment of the present invention, the default parameters can be used, with the expected value (evalue) being set to 1000, so that the software will retain all the sequences with expected values being less than or equal to 1000.

A description of BLAT (i.e., the “BLAST-like alignment tool”) software can be found in the literature: “Kent, W James (2002). BLAT—the BLAST-like alignment tool. Genome Research. 12(4): 656-664.”, the entire content of which is incorporated herein by reference. In one embodiment of the invention, default parameters may be adopted when using the software BLAT.

A description of the Wise2DBA software can be found in the literature: “Jareborg N, Birney E, Durbin R. Comparative analysis of noncoding regions of 77 orthologous mouse and human gene pairs. Genome Research 9: 815-824, 1999, the entire content of which is incorporated herein by reference. In one embodiment of the invention, default parameters may be adopted when using the software Wise2DBA.

Preferably, for each siRNA, the sense strand and the antisense strand are aligned with the sequences of genomic mRNAs, respectively.

Preferably, the characteristic of the mismatched bases comprises the number of mismatched bases, and optionally, the location of the mismatched bases.

Preferably, the secondary structural characteristic of the off-target gene's mRNA sequence is a probability of the mRNA itself not forming a secondary structure in the complementary region. The secondary structure of the mRNA in the complementary region can affect the probability of binding of the mRNA to the complementary siRNA in that region.

Preferably, for each of the selected off-target genes, the interference rate of the siRNA on the expression level of the off-target gene's mRNA is calculated according to the characteristic of the mismatched bases, and then the product of the interference rate and the probability of not forming the secondary structure is calculated, thereby obtaining the off-target weight of the off-target gene.

If a particular off-target gene's mRNA has multiple complementary regions to the sequence of the same siRNA, then the maximum of the off-target weights calculated for individual complementary regions is taken.

Different degrees of sequence matching between siRNA and mRNA result in different interference rates. For example, as the number of mismatched bases increases, the interference rate will decrease. Generally, if the number of mismatched bases reaches 7 or more, the interference rate of siRNA on the expression level of mRNA is negligible. The interference rate of siRNA on the expression level of mRNA can be determined theoretically or by biological experiments.

For example, the following method can be used to determine the interference rate of siRNAs with different numbers of mismatched bases for a given mRNA on the expression level of the mRNA, respectively. The expression level of a given mRNA in suitable cells is detected by qRT-PCR (hereinafter referred as the natural expression amount). siRNAs having different numbers of mismatched bases with the given mRNA are respectively transfected into the cells, and the mRNA expression levels under the respective mismatching conditions are detected by qRT-PCR method (hereinafter referred as interference expression level), followed by calculating the ratio of each interference expression level to the natural expression level and subtracting this ratio from 1 to obtain the interference rate of siRNA with different number of mismatched bases.

In addition, the present invention comprises performing a curve fitting process on the interference rate of siRNAs having different numbers of mismatched bases. It have been found that a nonlinear fitting formula can be obtained, and the fitting formula can be used to calculate the interference rate of siRNA having different number of mismatched bases with a specific mRNA on the expression level of the mRNA. The interference rate calculated by the fitting formula is highly close to the actual interference rate, and the accuracy is good.

In one embodiment of the invention, the nonlinear fitting formulas are as follows: 1) for the mismatched bases at the 3′ end: y_{3′}=−0.01316x_{3′}²−0.03245x_{3′}+1.0238; where x_{3′} is the number of mismatched bases at the 3′ end, and y_{3′} is the interference rate at the 3′ end; 2) for the mismatched base at the 5′ end: y_{5′}=−0.01313x_{5′}²+0.03223x_{5′}+0.95513, where x_{5′} is the number of mismatched bases at the 5′ end, and y_{5′} is the interference rate at the 5′ end. The method for obtaining the nonlinear fitting formula of the present invention may be, for example, as described in the Experimental Example 1 hereinafter. Although the nonlinear formula in Experimental Example 1 is obtained using the human MGMT gene (O-6-Methylguanine-DNA Methyltransferase) as the off-target gene, the linear fitting formula of the present invention is not limited to this gene and can be applied to other off-target genes.

Further, the nonlinear fitting formula of the present invention can be further optimized according to, for example, the method described in Experimental Example 1 hereinafter to improve the accuracy of the coefficient of the nonlinear formula.

The phrase “calculating an interference rate of the siRNA on the expression level of the off-target gene's mRNA according to the characteristic of the mismatched bases” in the present invention means the overall interference rate of the siRNA on the off-target gene, that is, y=y_{3′}×y_{5′}. For example, if a specific off-target gene has 2 mismatches at the 3′ end of the sense strand and 3 mismatches at the 5′ end of the sense strand in the region matching the siRNA, then the overall interference rate of the siRNA on the off-target gene is the product of the interference rates at both ends, i.e., 0.9060 times 0.9337 equals 0.8459.

In the method of the invention, the probability of the mRNA of each off-target gene not forming a secondary structure can be predicted using a software selected from the group consisting of: RNAPLFOLD, mfold and RNAstructure. When using these softwares, one can set the parameters as needed. A description of the RNAPLFOLD software can be found in the literature: “Lewis B P, Burge C B, Bartel D P. Conserved seed pairing, often flanked by adenosines, indicates that thousands of human genes are microRNA targets. Cell. 2005, 120(1): 15-20.”, the entire content of which is incorporated herein by reference. In one embodiment of the present invention, RNAPLFOLD software can be used to predict the secondary structure of human whole genome mRNA, and the output results can be integrated to form a localized database for high-speed reading and calculation. The parameter design of RNAPLFOLD can be: L=40, W=80, and u=25. Thereby, the probability of the off-target gene not forming a secondary structure is obtained.

In combination with the above-mentioned overall interference rate of the siRNA on the off-target gene, the off-target weight of the off-target gene is a product obtained by multiplying the probability of not forming the secondary structure and the overall interference rate.

In the step iii), the omic weight may be one, two or all selected from the group consisting of protein interaction weight, signal pathway weight, and core gene weight of the off-target gene.

Protein interaction weight can be obtained by omic annotation with respect to each of the selected off-target genes using the protein interaction network database “STRING”. “STRING” is one of the most authoritative databases of protein interaction networks in the world, covering the interaction data of known and predicted proteins (see “Szklarczyk D, Franceschini A, Kuhn M, Simonovic M, Roth A, Minguez P, Doerks T, Stark M, Muller J, Bork P, Jensen LJ, von Mering C. The STRING database in 2011: functional interaction networks of proteins, globally integrated and scored. Nucleic Acids Res. 2011, 39 (Database issue): D561-8.”, the entire content of which is incorporated herein by reference). These interactions include both physically direct effects and functionally indirect effects. These data are derived from genomic information, high-throughput biological experiments, conservative co-expression characteristics and literature disclosures. STRING organically quantifies and integrates the above-mentioned basic data. In a particular species, each pair of interacting proteins is weighted (weights ranging from 0 to 1000) to show the closeness of the association. If a protein participates in multiple pairs of interactions, then the protein's interaction weight is the sum of the weights of the interactions it participates in.

Signal pathway weight can be obtained by omic annotation with regard to each of the selected off-target genes using, for example, the human pathwayomic database “ConsensusPathDB-human” (see the literature: “Kamburov A, Pentchev K, Galicka H, Wierling C, Lehrach H, Herwig R. ConsensusPathDB: toward a more complete picture of cell biology. Nucleic Acids Res. 2011, 39 (Database issue): D712-7.”, the entire content of which is incorporated herein by reference). The database involves gene regulation, protein action, signal transduction, metabolism, drug targeting, biochemical reactions, etc. It is by far the most complete public pathwayomic database. For any one of the selected off-target genes, the number of pathways in which it participates can be extracted according to the database as the signal pathway weight.

As to core gene weights, it is known that the research team at the Department of Molecular Genetics at the University of Toronto used the latest gene editing technology, CRISPR, to shut down 18,000 genes (90% of the human genome) and found that more than 1,500 genes are essential for human (see literature: “Hart T, Chandrashekhar M, Aregger M, Steinhart Z, Brown KR, MacLeod G, Mis M, Zimmermann M, Fradet-Turcotte A, Sun S, Mero P, Dirks P, Sidhu S, Roth F P, Rissland O S, Durocher D, Angers S, Moffat J. High-Resolution CRISPR Screens Reveal Fitness Genes and Genotype-Specific Cancer Liabilities. Cell. 2015, 163(6): 1515-26.”, the entire content of which is incorporated herein by reference). Herein, the genes necessary for human are called “core genes.” If the selected off-target gene is a core gene, the toxic effect of the siRNA on the cells may be greater. For any one of the selected off-target genes, if it is a core gene, its core gene weight can be set to 1; otherwise, its core gene weight can be set to zero.

In the step iv), the omic eigenvalue may be one, two or all selected from the group consisting of proteomic eigenvalue, signal pathwayomic eigenvalue, and core genomic eigenvalue. The proteomic eigenvalue, the signal pathwayomic eigenvalue and the core genomic eigenvalue may be calculated according to the following a) to c), respectively:

a) calculating a product a′ of the off-target weight of each of the selected off-target genes and its protein interaction weight, and then calculating a sum of all the products a′ obtained for each of the selected off-target genes to generate a proteomic eigenvalue;

b) calculating a product b′ of the off-target weight of each of the selected off-target genes and its signal pathway weight, and then calculating a sum of all the products b′ obtained for each of the selected off-target genes to generate a signal pathwayomic eigenvalue;

c) calculating a product c′ of the off-target weight of each of the selected off-target genes and its core gene weight, and then calculating a sum of all the products c′ obtained for each of the selected off-target genes to generate a core genomic eigenvalue.

Preferably, the input values are normalized prior to establishing the machine learning model. The normalization process is to avoid the impact of a certain type of data on the establishment of the model in case the absolute value is too large. Usually, the formula, (a value-minimum)/(maximum-minimum), is used to map data to the interval 0-1, which is one of the commonly used classical methods.

The output values can also be binarized before the machine learning model is established, but this is not required. A certain cell survival index can be used as the boundary value. If a survival index is higher than or equal to this boundary value, it can be set to 1, and the rest can be set to zero. The cell survival index as a boundary value may be greater than or equal to 0.75. For example, when a cell survival index of 0.9 is used as a boundary value, a value higher than or equal to 0.9 is set to 1, and the rest is set to zero.

Preferably, the machine learning algorithm includes a support vector machine, an artificial neural network, a decision tree and a regression model. These machine learning algorithms can be implemented on the basis of integrated development softwares such as languages C, Perl, Python, R, and KNIME, and parameters can be set as needed. For example, when using the support vector machine algorithm to establish a machine learning model, the library function “svm” of R can be used, and the main parameter, kernel (function mapping mode for determining the data space), is set to linear, polynomial, radial, or sigmoid, with the linear being preferred. When the artificial neural network algorithm is used to establish the machine learning model, the library function “neuralnet” of R can be used to debug the main parameter, hidden (i.e., the number of hidden neurons/layers), which is preferably set to 1.

The established machine learning model can be evaluated using known evaluation methods. The most common method is cross validation. For example, it can be a 8-fold cross-validation, a 9-fold cross-validation, a 10-fold cross-validation, and the like.

Preferably, based on the principle of action of the siRNA, the selected off-target gene does not include such an off-target gene that a complementary region of its mRNA to the siRNA sequence is located only in the 5′ untranslated region (UTR).

The interference effect of siRNA is embodied in the silencing effect on the target gene. If, in a certain type of cells, a particular gene is not expressed by itself in a natural state, the interference of siRNA to this gene can be neglected. Therefore, preferably, based on the expression profile database of a known cell line, the selected off-target gene does not include a gene that is not expressed in a natural state (or in a normal state) in the certain type of cells. The expression profile database of the cell line is, for example, “THE HUMAN PROTEIN ATLAS” database (see the literature: “Uhlen M, Oksvold P, Fagerberg L, Lundberg E, Jonasson K, Forsberg M, Zwahlen M, Kampf C, Wester K, Hober S, Wernerus H, Bjorling L, Ponten F. Towards a knowledge-based Human Protein Atlas. Nat Biotechnol. 2010, 28(12): 1248-50.”, the entire content of which is hereby incorporated by reference). The database contains expression data for protein-coding genes from common cell lines, which are double validated at the RNA and protein levels, respectively.

In the method of the present invention, siRNA for performing experiments on cells can be prepared by a conventional method in the art, including, for example, chemical synthesis, in vitro transcription, siRNA expression vector, siRNA framework, and the like.

II. Application of the Method of the Invention in Predicting the Toxicity of siRNA to a Type of Cells

Another aspect of the invention also provides the use of the method of the invention for predicting the toxicity of siRNA to a certain type of cells.

III. Computer Readable Medium

Another aspect of the present invention also provides a computer readable medium useful for establishing the machine learning model in accordance with the method of the present invention, the computer readable medium comprising the following modules:

a sequence alignment module for performing the step i) in the method of the present invention;

an off-target weight calculation module for performing the step ii) in the method of the present invention;

an omic annotation module for performing the step iii) in the method of the present invention;

an omic eigenvalue calculation module for performing the steps iv) in the method of the present invention; and

a machine learning algorithm calculation module for performing the step C) in the method of the present invention.

The computer readable medium can include an external data input module for inputting n siRNA sequences and the corresponding cell survival indices, respectively.

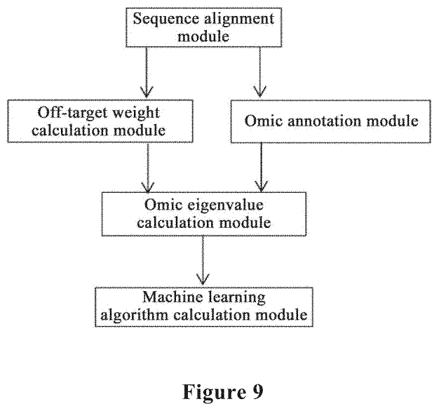

By way of example, FIG. 9 shows a schematic diagram of one embodiment of a computer readable medium of the present invention.

IV. Device for Predicting the Toxicity of siRNA to a Certain Type of Cells

Another aspect of the invention also provides a device for predicting the toxicity of an siRNA to a certain type of cells, comprising:

1) an input unit for inputting a sequence of the siRNA to be tested;

2) a storage unit for storing a machine learning model established for the type of cells using the method of the present invention;

3) an execution unit for executing the machine learning model on the sequence of the siRNA; and

4) an output unit for displaying a predicted result of the toxicity of the siRNA to the type of cells.

The device may be a device specially constructed for the purpose of the present invention, or may be a computer.

The input unit is, for example, but not limited to, a keyboard, a mouse, a scanner, or a touch screen, as is known in the art.

In one aspect of the invention, the storage unit can be any type of memory for storing data and/or software, including electrically programmable read only memory (EPROM), electrically erasable programmable read only memory (EEPROM), a virtual storage location on a network, a memory device, a computer readable medium, a computer disk, and a storage device that can transmit information, or any other type of media suitable for storing the machine learning model.

The output unit includes, but is not limited to, any type of displays and printers.

V. Method for Predicting the Toxicity of siRNA to a Certain Type of Cells

Another aspect of the invention also provides a method of predicting the toxicity of an siRNA to a certain type of cells, comprising:

providing a sequence of the siRNA to be tested;

inputting the sequence of the siRNA to the device of the invention, and allowing the device to execute the machine learning model established for the certain type of cells using the method according to the method of the invention, thereby obtaining result of the prediction of the toxicity of the siRNA to the certain type of cells.

The siRNA to be tested may be a drug candidate for antiviral (including respiratory virus, Ebola virus, etc.) infection. Generally, such siRNA sequences can be obtained by any means commonly used in the art. For example, the siRNA sequences to be tested are designed using known public or commercial siRNA design tools (e.g., Invitrogen, GenScript, Dharmacon, and/or siDirect, etc.) according to siRNA design principles well-known in the art.

An example of the siRNA design principles is to start at 50-100 bases after the gene promoter in the conserved region of the whole gene sequence of human respiratory virus, for example, to find a 19-21 bp (e.g., 19 bp) nucleotide sequence in the gene sequence that meets the following conditions: (1) starting with G or C, and ending with A or T; (2) at least 5 of the last 7 bases of the end are A or T; (3) avoiding 4 consecutive bases like AAAA or CCCC, thereby increasing the complexity of the bases; and/or (4) GC content between 30% and 52%.

The whole gene sequence of human respiratory virus includes a whole gene sequence of a known human respiratory virus or a new human respiratory virus. The whole gene sequence of a known human respiratory virus can be directly obtained from the public database Genebank, and the whole gene sequence of a new human respiratory virus can be obtained by isolating and extracting RNA, for example, and determining the sequence, and optionally, further genotyping by any known methods.

Preferably, in the method of the present invention, the respiratory virus includes an influenza virus, parainfluenza virus, respiratory syncytial virus, measles virus, mumps virus, adenovirus, rubella virus, rhinovirus, coronavirus and/or reovirus; more preferably an influenza virus; further preferably an influenza A virus; still more preferably an H1, H3, H5, H7 or H9 influenza A virus; and still more preferably H1N1, H3N2, H5N1, H7N7, H7N9 influenza A virus.

The present invention will be further explained or illustrated by way of examples, but the examples are not to be construed as limiting the scope of the invention.

### EXAMPLES

An embodiment of the invention is described below by referring to an example in which a machine learning model for predicting the toxicity of siRNA to human respiratory cells was established.

[Materials Used in the Experiment]

1) Materials for cell cultivation

A conventional culture solution was DMEM medium (Gibco, USA) supplemented with 10% (v/v) fetal bovine serum (Hyclone, USA). DMSO was purchased from Sigma-Aldrich, USA.

2) qRT-PCR detection related reagents

The total RNA extraction kit, reverse transcription kit and fluorescent quantitative PCR kit were purchased from Promega Company, USA.

Transfection reagent liposome, lipo2000, was purchased from Invitrogen, USA; and all the siRNA sequences were synthesized in Invitrogen, USA.

3) Cell survival index related reagent

The CCK-8 kit (containing CCK-8 solution) was purchased from DOJINDO, Japan.

4) Experimental consumables

The disposable experimental consumables used in the experiment were purchased from Corning, USA.

Unless otherwise stated, the following biological experiments were carried out using conventional methods, materials, conditions and equipment known in the art.

### Experimental Example 1: Interference Rate of siRNA on the Expression Level of Off-Target Gene's mRNA

Different levels of sequence matching between siRNA and mRNA would lead to different interference effects, and the specific weights were set according to biological experimental data. The non-small cell lung cancer cell line A549 and the human gene MGMT (O-6-Methylguanine-DNA Methyltransferase), which would weakly expressed in the A549 cell line as known in the art, were selected. The weakly expressed gene was chosen because in the case of a strongly expressed gene, large doses of siRNA may be required to detect interference, and large doses of exogenous siRNA may cause other immune stimuli and element saturation effect.

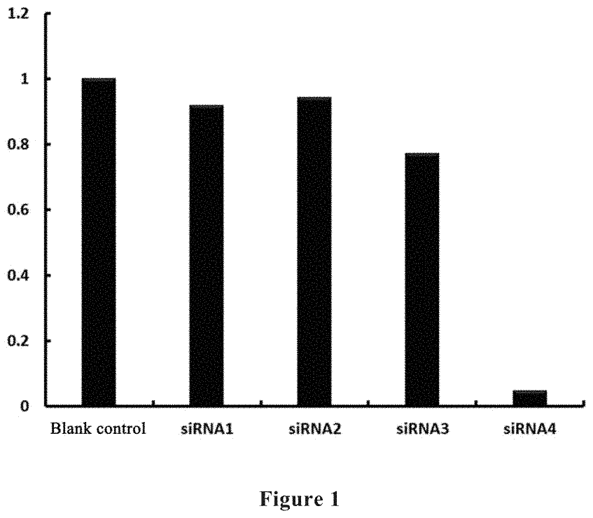

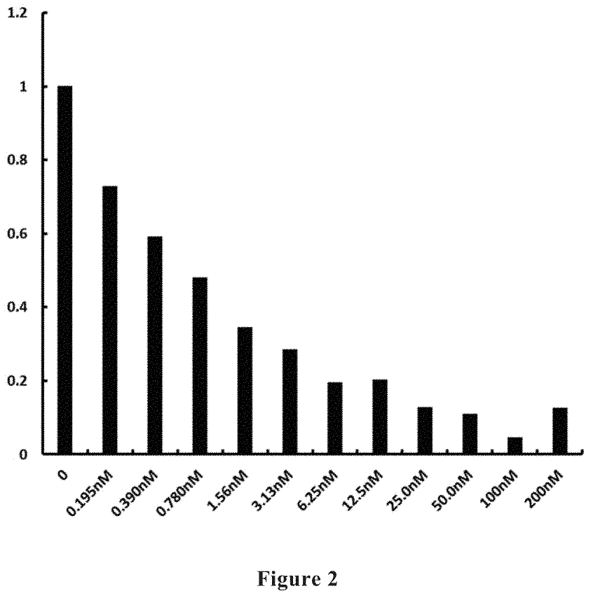

For MGMT, four siRNA sequences were designed (each siRNA consisting of a sense strand sequence and an antisense strand sequence in a pair), as shown in Table 1. The A549 cells were transfected with siRNA at a concentration of 50 nM, and the untransfected blank group was used as a control. The cells were cultured in a complete medium (10% FBS+90% DMEM: F12 (1:1)) at 37° C. in a 5% CO₂ incubator for 48 hours, and then detected by qRT-PCR method to determine the mRNA expression level of MGMT. The results are shown in FIG. 1, wherein the mRNA expression level of MGMT in the blank group was set to 1, and the mRNA expression levels in other transfected groups were relative percentages. The mRNA expression level of MGMT in the siRNA4 group was <10%, that is, the siRNA4 interference effect was >90%, which was determined to be an effective interference sequence. Thereafter, the effective interference sequence was used to explore the optimal siRNA transfection concentration, and FIG. 2 shows the respective transfection concentrations tested. As shown in FIG. 2, the transfection concentration was almost saturated at 25 nM, and thus the transfection concentrations of the subsequent experiments were selected to be 25 nM.

TABLE 1
siRNA designed for MGMT gene

| siRNA | Sense sequence | Anti-sense sequence |
|---|---|---|
| name | (SEQ ID) | (SEQ ID) |
| siRNA1 | GGAAGCCUAUUUCCGUGAATT | UUCACGGAAAUAGGCUUCCTT |
| | (SEQ ID NO: 1) | (SEQ ID NO: 2) |
| siRNA2 | GACAAGGAUUGUGAAAUGATT | UCAUUUCACAAUCCUUGUCTT |
| | (SEQ ID NO: 3) | (SEQ ID NO: 4) |
| siRNA3 | AUGGCUUCUGGCCCAUGAATT | UUCAUGGGCCAGAAGCCAUTT |
| | (SEQ ID NO: 5) | (SEQ ID NO: 6) |
| siRNA4 | CCAGACAGGUGUUAUGGAATT | UUCCAUAACACCUGUCUGGTT |
| | (SEQ ID NO: 7) | (SEQ ID NO: 8) |

Based on the selected effective interference sequences, 15 mismatched sequences were synthesized, as shown in Table 2, wherein the underlined portions were mismatched bases.

TABLE 2
Sequence design of mismatched siRNA for MGMT gene

| | Sense sequence | Anti-sense sequence |
|---|---|---|
| siRNA name | (SEQ ID) | (SEQ ID) |
| siRNA5 | CCAGACAGGUGUUAUGGAUTT | AUCCAUAACACCUGUCUGGTT |
| | (SEQ ID NO: 9) | (SEQ ID NO: 10) |
| siRNA6 | CCAGACAGGUGUUAUGGUUTT | AACCAUAACACCUGUCUGGTT |
| | (SEQ ID NO: 11) | (SEQ ID NO: 12) |
| siRNA7 | CCAGACAGGUGUUAUGCUUTT | AAGCAUAACACCUGUCUGGTT |
| | (SEQ ID NO: 13) | (SEQ ID NO: 14) |
| siRNA8 | CCAGACAGGUGUUAUCCUUTT | AAGGAUAACACCUGUCUGGTT |
| | (SEQ ID NO: 15) | (SEQ ID NO: 16) |
| siRNA9 | CCAGACAGGUGUUAACCUUTT | AAGGUUAACACCUGUCUGGTT |
| | (SEQ ID NO: 17) | (SEQ ID NO: 18) |
| siRNA10 | CCAGACAGGUGUUUACCUUTT | AAGGUAAACACCUGUCUGGTT |
| | (SEQ ID NO: 19) | (SEQ ID NO: 20) |
| siRNA11 | CCAGACAGGUGUAUACCUUTT | AAGGUAUACACCUGUCUGGTT |
| | (SEQ ID NO: 21) | (SEQ ID NO: 22) |
| siRNA12 | GCAGACAGGUGUUAUGGAATT | UUCCAUAACACCUGUCUGCTT |
| | (SEQ ID NO: 23) | (SEQ ID NO: 24) |
| siRNA13 | GGAGACAGGUGUUAUGGAATT | UUCCAUAACACCUGUCUCCTT |
| | (SEQ ID NO: 25) | (SEQ ID NO: 26) |
| siRNA14 | GGUGACAGGUGUUAUGGAATT | UUCCAUAACACCUGUCACCTT |
| | (SEQ ID NO: 27) | (SEQ ID NO: 28) |
| siRNA15 | GGUCACAGGUGUUAUGGAATT | UUCCAUAACACCUGUGACCTT |
| | (SEQ ID NO: 29) | (SEQ ID NO: 30) |
| siRNA16 | GGUCUCAGGUGUUAUGGAATT | UUCCAUAACACCUGAGACCTT |
| | (SEQ ID NO: 31) | (SEQ ID NO: 32) |
| siRNA17 | GGUCUGAGGUGUUAUGGAATT | UUCCAUAACACCUCAGACCTT |
| | (SEQ ID NO: 33) | (SEQ ID NO: 34) |
| siRNA18 | GGUCUGUGGUGUUAUGGAATT | UUCCAUAACACCACAGACCTT |
| | (SEQ ID NO: 35) | (SEQ ID NO: 36) |
| siRNA19 | CCAGACAGCACUUAUGGAATT | UUCCAUAAGUGCUGUCUGGTT |
| | (SEQ ID NO: 37) | (SEQ ID NO: 38) |

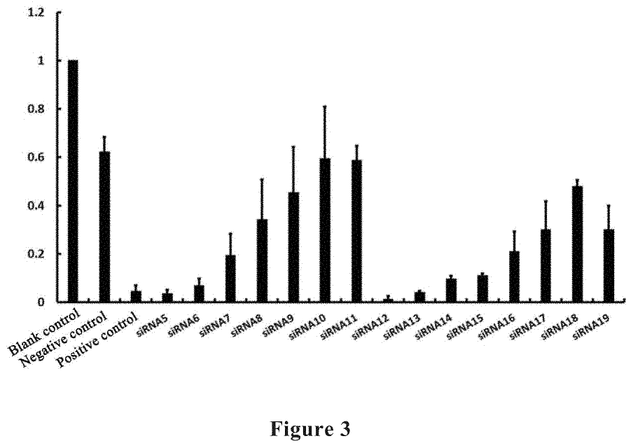

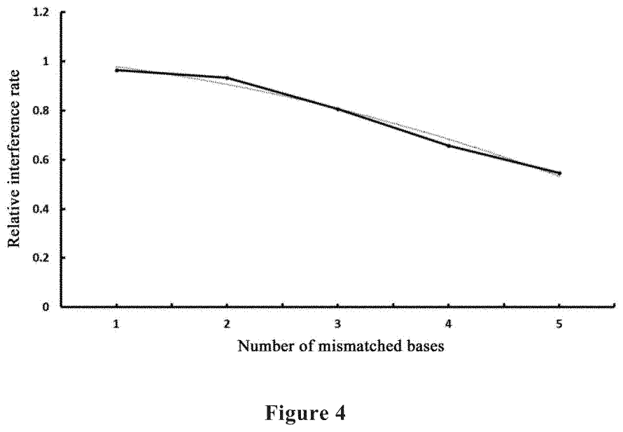

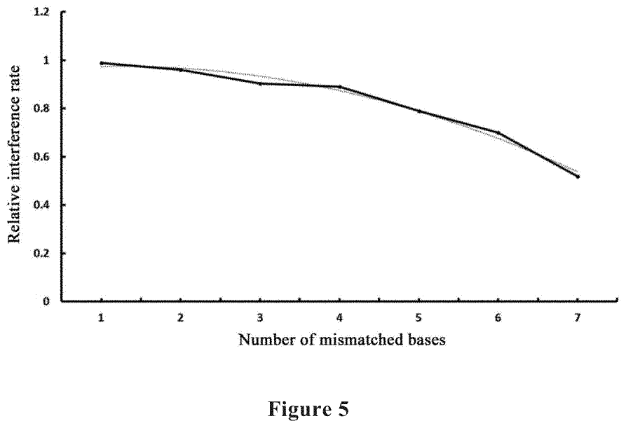

A549 cells were transfected with these siRNAs. There were also a blank group (untransfected), a negative control group (transfected with a random siRNA sequence (synthesized by Invitrogen), i.e., an siRNA not targeting at MGMT gene), and a positive control group (transfected with an siRNA capable of efficiently knocking out the MGMT, i.e., siRNA4). After cultured for 48 hours under the culture conditions as described above, the effect of siRNA of each mismatched sequence on the mRNA level of MGMT was examined by qRT-PCR. The results are shown in FIG. 3. All the expression levels of mRNA are relative to the blank control group. It shows that, as the number of mismatched bases increases, the expression level of mRNA also increases, that is, the interference effect of siRNA is reduced. This applies no matter whether the mismatched bases are located at the 5′ or 3′ end, differing only in the weight coefficient (interference rate). Based on the mRNA expression data, the interference rate of the siRNA were obtained by calculating the ratio of the expression level of each experimental group to that of the blank control group, and subtracting the ratio from 1. The interference rates of these siRNAs were subjected to curve fitting processing. Since the expression level of mRNA in the negative control group was about 0.6, and the expression levels of mRNA in the siRNA10 group and the siRNA11 group were also close to 0.6, they were not included in the curve fitting process. The fitting curves are shown in FIGS. 4 and 5. The nonlinear fitting formulas for the mismatches at the 3′ end (FIG. 4) and the 5′ end (FIG. 5) are respectively as follows:

1) for the mismatched bases at the 3′ end: y_{3′}=−0.01316x_{3′}²−0.03245x_{3′}+1.0238; where x_{3′} is the number of mismatched bases at the 3′ end, and y_{3′} is the interference rate at the 3′ end;

2) for the mismatched base at the 5′ end: y_{5′}=−0.01313x_{5′}²+0.03223x_{5′}+0.95513, where x_{5′} is the number of mismatched bases at the 5′ end, and y_{5′} is the interference rate at the 5′ end.

The overall interference rate of the siRNA on the off-target gene is expressed by y=y_{3′}×y_{5′}.

### Example 1: Procedure for Establishing a Machine Learning Model for Predicting the Toxicity of siRNA to Human Respiratory Cells

A. Providing siRNAs for Establishing a Machine Learning Model

The above 16 siRNAs (siRNA4 in Table 1 and 15 mismatched sequences, siRNA5-siRNA 19, in Table 2) were used to establish a machine learning model.

B. Obtaining Input and Output Values for Establishing a Machine Learning Model

Among them, the input values of any of the 16 siRNAs were obtained as follows:

i) aligning siRNA sequences with human genomic mRNA sequences, and further screening off-target genes based on functional annotation and expression profile database.

In order to preliminarily determine the off-target gene of a certain siRNA, a localized mRNA sequence database of the human genome (that is, downloading the mRNA sequences to a hard disk, such that subsequent work could be done independently of the network) was established by BLAST (version number 2.2.31) software (see the literature: “Camacho C, Coulouris G, Avagyan V, Ma N, Papadopoulos J, Bealer K, Madden TL. BLAST+: architecture and applications. BMC Bioinformatics. 2008, 10:421.”). The sequence of the siRNA and the mRNA sequence data of the human genome were comprehensively aligned. In order to obtain comprehensive alignment results, but not just highly similar alignment, in the BLAST software the blastn mode was chosen. Most of the parameter settings of the BLAST software adopted the default parameters, as follows: evalue=1000, word_size=7, gapopen=5, gapextend=2, penalty=3, reward=2. During alignment, the sense and antisense strands of the siRNA were aligned, respectively.

By alignment, a complete preliminary off-target gene list was obtained, and then the region where the siRNA and each off-target gene's mRNA match was functionally annotated as to whether the action region of the siRNA was distributed in the 5′ UTR, 3′ UTR, or coding region of the mRNA. Based on the principle of siRNA's action, only such an off-target gene that the siRNA matching site was located in the 3′ UTR and/or coding region of its mRNA was concerned in the subsequent analysis.

The off-target gene that was not expressed by itself in human respiratory cells (for example, non-small cell lung cancer cell line A549) was deleted from the off-target gene list, using the expression profile database of the known cell line. The expression profile data for the cell line was derived from the “THE HUMAN PROTEIN ATLAS” database.

A series of off-target genes were thus selected. For each of the 16 siRNAs, hundreds of off-target genes were obtained. The specific statistical results of the number of off-target genes are shown in Table 3.

TABLE 3
Statistics on the number of off-target genes of siRNAs

| siRNA name | Number of off-target genes |
|---|---|
| siRNA4 | 138 |
| siRNA5 | 131 |
| siRNA6 | 140 |
| siRNA7 | 124 |
| siRNA8 | 131 |
| siRNA9 | 120 |
| siRNA10 | 134 |
| siRNA11 | 101 |
| siRNA12 | 132 |
| siRNA13 | 136 |
| siRNA14 | 121 |
| siRNA15 | 127 |
| siRNA16 | 129 |
| siRNA17 | 121 |
| siRNA18 | 151 |
| siRNA19 | 151 |

ii) Determining the off-target weights of the selected off-target genes

The interference rate of the curve fitting obtained in Experimental Example 1 was used as a standard, and weights were set for the respective off-target genes.

For example, if the matched region of a specific off-target gene, human ERCC6 (Excision Repair Cross-Complementation 6), with a specific siRNA, e.g., siRNA4 (sense strand sequence CCAGACAGGUGUUAUGGAATT (SEQ ID NO: 7)), has 1 mismatch at the 3′ end of the sense strand and 5 mismatches at the 5′ end of the sense strand, then the overall interference rate of the siRNA on the off-target gene is the product of interference rates at both ends, i.e., 0.9782 times 0.7880 is equal to 0.7708.

For the complementary region, the software RNAPLFOLD (version 2.2.4) (see the literature: “Lewis B P, Burge C B, Bartel D P. Conserved seed pairing, often flanked by adenosines, indicates that thousands of human genes are microRNA targets. Cell. 2005, 120(1): 15-20.”) was used to determine the probability of the off-target gene's mRNA itself not forming a secondary structure. Specifically, the software was used to predict the secondary structure of human whole genome mRNA and extract the relevant text and numerical information from the output to form a localized database for high-speed reading and improvement of calculation speed. The parameter design of RNAPLFOLD includes: L=40, W=80, u=25. For example, in the region where the mRNA of the off-target gene was complementary to the siRNA sequence, the probability of the off-target gene not forming a secondary structure was 0.5425, and the overall interference rate, obtained based on the interference rates at both ends, was 0.7708, such that the off-target weight of the off-target gene was 0.5425×0.7708=0.4182.

iii)-vi) obtaining omic weights based on omic annotation of the selected off-target genes; and calculating omic eigenvalues from the omic weights and off-target weights

(1) Calculating Proteomic Eigenvalues Based on the Protein Interaction Weights and Off-Target Weights of all the Selected Off-Target Genes

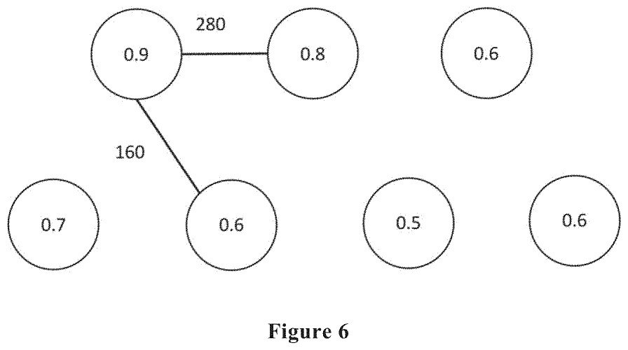

The human LINKS table in the STRING database was localized (that is, downloaded to the hard disk of a local computer), and the names of proteins were converted into common gene names for calculation operations. Cells were treated with a specific siRNA, and the possible off-target genes and their weights were determined by the methods described above. FIG. 6 illustratively shows a simplified example of a certain siRNA having seven off-target genes (represented by circles), each of which has an exemplary off-target weight (the number in the circle). Based on the information in the STRING database, those genes having interactions and the protein interaction weights thereof were determined. FIG. 6 exemplarily shows an example in which there are interactions among three genes that are connected by lines, with the numbers on the lines showing the weights of the interaction. Thus, the proteomic eigenvalue was calculated as follows: proteomic eigenvalue=0.9×(280+160)+0.8×280+0.6×160. That is, for each off-target gene, if it participates in an interaction, its off-target weight is multiplied by the protein interaction weight; if it participates in multiple interactions, its off-target weight is multiplied by the sum of the respective protein interaction weights; if the off-target gene is isolated, its effect is ignored. The results of calculation of proteomic eigenvalues of the off-target genes of the respective siRNAs are shown in Table 4.

TABLE 4
Results of calculation of proteomic eigenvalues
of off-target genes of siRNAs

| siRNA name | Proteomic eigenvalues |
|---|---|
| siRNA4 | 150.0129 |
| siRNA5 | 135.2095 |
| siRNA6 | 182.5355 |
| siRNA7 | 97.8546 |
| siRNA8 | 102.6913 |
| siRNA9 | 88.8456 |
| siRNA10 | 106.3368 |
| siRNA11 | 65.3091 |
| siRNA12 | 141.7128 |
| siRNA13 | 101.5539 |
| siRNA14 | 82.2402 |
| siRNA15 | 107.9213 |
| siRNA16 | 107.9795 |
| siRNA17 | 122.7014 |
| siRNA18 | 182.3832 |
| siRNA19 | 134.8214 |

(2) Calculating Signal Pathwayomic Eigenvalues Based on Signal Pathway Weights and Off-Target Weights of all the Selected Off-Target Genes.

The human pathway database ConsensusPathDB-human (version number 31) was localized. By multiplying the determined off-target weight of each off-target gene and the number of pathways involved, and then calculating the sum, the signal pathwayomic eigenvalue was obtained. If the off-target gene was isolated, its effect was ignored. For example, three off-target genes A, B, and C were identified. According to the database, A was involved in 3 known pathways, B was involved in 2 known pathways, and C was isolated. Then, their signal pathwayomic eigenvalue was calculated as follows: (the off-target weight of A multiplied by 3) plus (the off-target weight of B multiplied by 2). The calculation results of the signal pathwayomic eigenvalues of the off-target genes of the respective siRNAs are shown in Table 5.

TABLE 5
Calculation results of signal pathwayomic
eigenvalues of off-target genes of siRNAs

| siRNA name | Signal pathwayomic eigenvalues |
|---|---|
| siRNA4 | 653.2424 |
| siRNA5 | 585.7767 |
| siRNA6 | 742.5516 |
| siRNA7 | 372.6335 |
| siRNA8 | 404.0694 |
| siRNA9 | 416.7108 |
| siRNA10 | 419.1286 |
| siRNA11 | 318.9158 |
| siRNA12 | 717.8643 |
| siRNA13 | 476.5563 |
| siRNA14 | 362.0600 |
| siRNA15 | 368.6291 |
| siRNA16 | 440.0923 |
| siRNA17 | 551.1228 |
| siRNA18 | 837.8167 |
| siRNA19 | 258.3346 |

(3) Calculating Core Genomic Eigenvalues Based on Core Gene Weights and Off-Target Weights of all the Selected Off-Target Genes

Currently, it is known that more than 1,500 core genes have been discovered. For example, if four off-target genes A′, B′, C′, and D′ were identified, among which B′ and C′ were determined as core genes based on the known core genes, then their core genomic eigenvalue was counted as the sum of off-target weights of B′ and C′. The calculation results of the core genomic eigenvalues of the off-target genes of the respective siRNAs are shown in Table 6.

TABLE 6
Calculation results of core genomic eigenvalues
of off-target genes of siRNAs

| siRNA name | Core genomic eigenvalues |
|---|---|
| siRNA4 | 7.6147 |
| siRNA5 | 6.6085 |
| siRNA6 | 8.0534 |
| siRNA7 | 5.7126 |
| siRNA8 | 8.5514 |
| siRNA9 | 5.3999 |
| siRNA10 | 5.8094 |
| siRNA11 | 4.6920 |
| siRNA12 | 7.3661 |
| siRNA13 | 5.4217 |
| siRNA14 | 5.7174 |
| siRNA15 | 4.9374 |
| siRNA16 | 6.1381 |
| siRNA17 | 4.1732 |
| siRNA18 | 7.2726 |
| siRNA19 | 4.0797 |

The output value of any of the 16 siRNAs was obtained as follows:

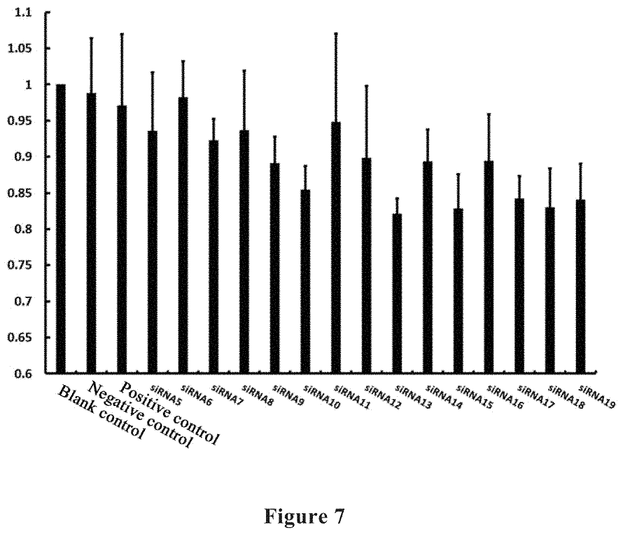

A549 cells were transfected with the above 16 siRNAs (siRNA4 in Table 1 and 15 mismatched sequences, siRNA5-siRNA19, in Table 2). There were also a blank group (untransfected), and a negative control group (transfected with a random siRNA sequence (synthesized by Invitrogen), i.e., an siRNA not targeting at MGMT gene). After cultured for 48 hours under the culture conditions as described above, the cells were treated with CCK-8 solution by adding 10 μL of CCK-8 solution to each well, and the plate was incubated in an incubator for 0.5-1 hour. The absorbance at 450 nm was measured by a microplate reader, and the OD450 data was collected. The ratio of the OD450 value of each experimental group to the OD450 value of the blank group was calculated, and thus the cell survival index of each group was obtained. The results are shown in FIG. 7.

By comparing FIG. 7 with FIG. 3, there is no significant correlation between the survival indexes of the cells transfected with siRNAs and the mRNA expression levels of MGMT, and there is no regular relationship between the survival indexes and the mismatched numbers or sites. This indicates that the difference in cell survival indexes is caused by the off-target effect of siRNAs. In addition to the off-target genes, siRNA also has a certain effect on other genes, because each off-target gene has complex network interaction effects at various levels such as RNAome, proteome, and pathwayome.

C. Establishing a Machine Learning Model Through Machine Learning Algorithm

(1) Establishing a Machine Learning Model Through the Machine Learning Algorithm ANN

As described above, the proteomic eigenvalue, the signal pathwayomic eigenvalue, and the core genomic eigenvalue were obtained for a specific siRNA. These data need to be normalized before being used as input values for machine learning algorithms. The data were mapped one-to-one to the interval 0-1 using the formula: (a value-minimum)/(maximum-minimum). The results of the normalized proteomic eigenvalues, signal pathwayomic eigenvalues, and core genomic eigenvalues are shown in Table 7.

TABLE 7
Results of proteomic eigenvalues, signal pathwayomic eigenvalues,
and core genomic eigenvalues after normalization

| | Normalized | Normalized | Normalized |
|---|---|---|---|
| | proteomic | signal pathwayomic | core genomic |
| siRNA name | eigenvalues | eigenvalues | eigenvalues |
| siRNA4 | 0.7226 | 0.6815 | 0.7905 |
| siRNA5 | 0.5963 | 0.5651 | 0.5655 |
| siRNA6 | 1.0000 | 0.8356 | 0.8886 |
| siRNA7 | 0.2776 | 0.1972 | 0.3652 |
| siRNA8 | 0.3189 | 0.2515 | 1.0000 |
| siRNA9 | 0.2008 | 0.2733 | 0.2952 |
| siRNA10 | 0.3500 | 0.2775 | 0.3868 |
| siRNA11 | 0.0000 | 0.1045 | 0.1369 |
| siRNA12 | 0.6518 | 0.7930 | 0.7349 |
| siRNA13 | 0.3092 | 0.3766 | 0.3001 |
| siRNA14 | 0.1444 | 0.1790 | 0.3662 |
| siRNA15 | 0.3635 | 0.1903 | 0.1918 |
| siRNA16 | 0.3640 | 0.3137 | 0.4603 |
| siRNA17 | 0.4896 | 0.5053 | 0.0209 |
| siRNA18 | 0.9987 | 1.0000 | 0.7140 |
| siRNA19 | 0.5930 | 0.0000 | 0.0000 |

For the output value data of the machine learning algorithm, that is, the survival indexes of the cells in the presence of siRNA, they were binarized before being used as the output value data (for example, with a survival index of 0.9 as the boundary value, those higher than or equal to 0.9 being set to 1, and the rest being set to 0). The cell survival index results after the binarization treatment are shown in Table 8.

TABLE 8
Cell survival index results after binarization

| siRNA name | Cell survival index results after binarization |
|---|---|
| siRNA4 | 1 |
| siRNA5 | 1 |
| siRNA6 | 1 |
| siRNA7 | 1 |
| siRNA8 | 1 |
| siRNA9 | 0 |
| siRNA10 | 0 |
| siRNA11 | 1 |
| siRNA12 | 0 |
| siRNA13 | 0 |
| siRNA14 | 0 |
| siRNA15 | 0 |
| siRNA16 | 0 |
| siRNA17 | 0 |
| siRNA18 | 0 |
| siRNA19 | 0 |

The normalized proteomic eigenvalues, signal pathwayomic eigenvalues and core genomic eigenvalues were taken as input values and the binarized cell survival indexes were taken as output values into an artificial network algorithm (ANN) The R library function, neuralnet, was used, wherein the main adjustable parameter was “hidden”, and the preferred setting thereof was 1.

The model was evaluated by 8-fold cross validation. The data set was divided into 8 parts, 7 of which were used for training and 1 for verifying in turn, and the average of 8 results was used as an estimate of the accuracy of the algorithm. The accuracy of the above algorithm can reach 56.25%.

(2) Establishing a Machine Learning Model Through the Machine Learning Algorithm SVM

As described above, proteomic eigenvalues, signal pathwayomic eigenvalues, and core genomic eigenvalues were obtained for a specific siRNA. These data need to be normalized before being used as input values for machine learning algorithms. The data were mapped one-to-one to the interval 0-1 using the formula: (a value-minimum)/(maximum-minimum). The results are identical to those reported in Table 7.

For the output value data of the machine learning algorithm, that is, the survival index of the cells in the presence of siRNA, it was binarized before being used as the output value data (for example, with a survival index of 0.9 as the boundary value, those higher than or equal to 0.9 being set to 1, and the rest being set to 0). The results are identical to those reported in Table 8.

The normalized proteomic eigenvalues, signal pathwayomic eigenvalues and core genomic eigenvalues were taken as input values and the binarized cell survival indexes were taken as output values into a support vector machine algorithm (SVM). The R library function, svm, was used, wherein the main adjustable parameter was “hidden”, and the preferred setting thereof was linear.

The model was evaluated by 8-fold cross-validation. The data set was divided into 8 parts. 7 of which were used for training and 1 for verifying in turn, and the average of 8 results was used as an estimate of the accuracy of the algorithm. The accuracy of the above algorithm can reach 62.5%.

In the present example, 16 siRNAs (i.e., n=16) were employed. It is to be understood that the accuracy of the above algorithms could be further improved when the sample size of the above siRNAs was increased.

### Example 2: Prediction of Toxicity of siRNA to Human Respiratory Cells Using the Machine Learning Model

As an example, the machine learning model obtained in Example 1 (specifically, the machine learning model established by the machine learning algorithm SVM) was used to predict the toxic effects of the above 16 siRNAs on the human respiratory cells. The results are shown in Table 9, wherein the values obtained by the experiment (the experimental values after binarization, that is, the cell survival index results after binarization as shown in Table 8) and the values predicted by the machine learning model (predicted values) are listed separately, and those predicted values that differ from the experimental values are underlined. The meanings of the numerical values in Table 9 are as follows: a cell survival rate of 0.9 is used as a boundary value, a value greater than 0.9 is set to 1, and a value less than 0.9 is set to 0, that is, 1 indicates no cytotoxicity, and 0 indicates cytotoxicity.

TABLE 9
Toxic effect of siRNA on human respiratory cells

| siRNA name | Binarized experimental value | Predicted value |
|---|---|---|
| siRNA4 | 1 | 0 |
| siRNA5 | 1 | 0 |
| siRNA6 | 1 | 1 |
| siRNA7 | 1 | 0 |
| siRNA8 | 1 | 1 |
| siRNA9 | 0 | 0 |
| siRNA10 | 0 | 0 |
| siRNA11 | 1 | 0 |
| siRNA12 | 0 | 0 |
| siRNA13 | 0 | 0 |
| siRNA14 | 0 | 0 |
| siRNA15 | 0 | 0 |
| siRNA16 | 0 | 0 |
| siRNA17 | 0 | 0 |
| siRNA18 | 0 | 0 |
| siRNA19 | 0 | 0 |

From the results shown in Table 9, it is known that the model established by the method of the present invention can more accurately predict those siRNAs which are relatively cytotoxic. In practical applications, those siRNAs with a predicted value of 1 (no cytotoxicity) can be selected as further drug candidates.

### Example 3: Procedure for Establishing a Machine Learning Model for Predicting the Toxicity of siRNA to Human Respiratory Cells

A. Providing siRNAs for Establishing a Machine Learning Model

The 180 siRNAs shown in Table 10 were used to establish a machine learning model.

TABLE 10
siRNA sequence information

| siRNA name | Sense strand sequence | Anti-sense strand sequence |
|---|---|---|
| siRNA_b2_1 | AUAUUCCUUAAGGGCUUCG | CGAAGCCCUUAAGGAAUAU |
| | (SEQ ID NO: 39) | (SEQ ID NO: 40) |
| siRNA_b2_2 | AUGAUCCAGACUGCAAUGC | GCAUUGCAGUCUGGAUCAU |
| | (SEQ ID NO: 41) | (SEQ ID NO: 42) |
| siRNA_b2_3 | AGUACAACCAAGGGUUUCC | GGAAACCCUUGGUUGUACU |
| | (SEQ ID NO: 43) | (SEQ ID NO: 44) |
| siRNA_b2_4 | AGAAAGACCCUUCAAUUCG | CGAAUUGAAGGGUCUUUCU |
| | (SEQ ID NO: 45) | (SEQ ID NO: 46) |
| siRNA_b2_5 | AAUAAAGUUGGCAGAGUCC | GGACUCUGCCAACUUUAUU |
| | (SEQ ID NO: 47) | (SEQ ID NO: 48) |
| siRNA_b2_6 | UCUGAAGGGAGAGAAAGAG | CUCUUUCUCUCCCUUCAGA |
| | (SEQ ID NO: 49) | (SEQ ID NO: 50) |
| siRNA_b2_7 | UAAGAUUCUGAAGGGAGAG | CUCUCCCUUCAGAAUCUUA |
| | (SEQ ID NO: 51) | (SEQ ID NO: 52) |
| siRNA_b2_8 | UCUUCUAAGAUCCAAAGCC | GGCUUUGGAUCUUAGAAGA |
| | (SEQ ID NO: 53) | (SEQ ID NO: 54) |
| siRNA_b2_9 | UAAUAGGGAUGGGCUCAAC | GUUGAGCCCAUCCCUAUUA |
| | (SEQ ID NO: 55) | (SEQ ID NO: 56) |
| siRNA_b2_10 | UUUCUGGGAAAGCUUGUAG | CUACAAGCUUUCCCAGAAA |
| | (SEQ ID NO: 57) | (SEQ ID NO: 58) |
| siRNA_b2_11 | UAUACUUGAGGCCACAGUC | GACUGUGGCCUCAAGUAUA |
| | (SEQ ID NO: 59) | (SEQ ID NO: 60) |
| siRNA_b2_12 | UCAAAUGAACGCCCAAUGC | GCAUUGGGCGUUCAUUUGA |
| | (SEQ ID NO: 61) | (SEQ ID NO: 62) |
| siRNA_b2_13 | UAACUUUCAGCUGGUCAUC | GAUGACCAGCUGAAAGUUA |
| | (SEQ ID NO: 63) | (SEQ ID NO: 64) |
| siRNA_b2_14 | UCAGUGUAGAAGUCAGCUG | CAGCUGACUUCUACACUGA |
| | (SEQ ID NO: 65 | (SEQ ID NO: 66) |
| siRNA_b2_15 | UGAGACAUCUGAUCCUUGG | CCAAGGAUCAGAUGUCUCA |
| | (SEQ ID NO: 67) | (SEQ ID NO: 68) |
| siRNA_b2_16 | AUUUUGGUCUGACUGCUUG | CAAGCAGUCAGACCAAAAU |
| | (SEQ ID NO: 69) | (SEQ ID NO: 70) |
| siRNA_b2_17 | AAUGGAGACAGUCAUGUAC | GUACAUGACUGUCUCCAUU |
| | (SEQ ID NO: 71) | (SEQ ID NO: 72) |
| siRNA_b2_18 | AUAAACAUGGCAGUGACAC | GUGUCACUGCCAUGUUUAU |
| | (SEQ ID NO: 73) | (SEQ ID NO: 74) |
| siRNA_b2_19 | UUUCUGGAGGGUACAUUUC | GAAAUGUACCCUCCAGAAA |
| | (SEQ ID NO: 75) | (SEQ ID NO: 76) |
| siRNA_b2_20 | UGUCCAUUCACCAUUAUCC | GGAUAAUGGUGAAUGGACA |
| | (SEQ ID NO: 77) | (SEQ ID NO: 78) |
| siRNA_b2_21 | UUUGAAGUAGGACACCGAG | CUCGGUGUCCUACUUCAAA |
| | (SEQ ID NO: 79) | (SEQ ID NO: 80) |
| siRNA_b2_22 | UGUAGAUGCACAGCUUCUC | GAGAAGCUGUGCAUCUACA |
| | (SEQ ID NO: 81) | (SEQ ID NO: 82) |
| siRNA_b2_23 | UGUUCAAUGAAAUCGUGCG | CGCACGAUUUCAUUGAACA |
| | (SEQ ID NO: 83) | (SEQ ID NO: 84) |
| siRNA_b2_24 | UCACACUUGAUCACUCUGG | CCAGAGUGAUCAAGUGUGA |
| | (SEQ ID NO: 85) | (SEQ ID NO: 86) |
| siRNA_b2_25 | UCUGGUAUCAAAAUGCUCC | GGAGCAUUUUGAUACCAGA |
| | (SEQ ID NO: 87) | (SEQ ID NO: 88) |
| siRNA_b2_26 | AUUAGGAUGGUUAAGCUCC | GGAGCUUAACCAUCCUAAU |
| | (SEQ ID NO: 89) | (SEQ ID NO: 90) |
| siRNA_b2_27 | UGUAAGUACGAACAGGGAC | GUCCCUGUUCGUACUUACA |
| | (SEQ ID NO: 91) | (SEQ ID NO: 92) |
| siRNA_b2_28 | AAUAUUUGCAGCCCAGGAG | CUCCUGGGCUGCAAAUAUU |
| | (SEQ ID NO: 93) | (SEQ ID NO: 94) |
| siRNA_b2_29 | AAUCUCAGAAUCUCCAGGG | CCCUGGAGAUUCUGAGAUU |
| | (SEQ ID NO: 95) | (SEQ ID NO: 96) |
| siRNA_b2_30 | UUACUAAAAUCUUGCCGGG | CCCGGCAAGAUUUUAGUAA |
| | (SEQ ID NO: 97) | (SEQ ID NO: 98) |
| siRNA_b2_31 | UUAGAAGGAGGAACUCCAG | CUGGAGUUCCUCCUUCUAA |
| | (SEQ ID NO: 99) | (SEQ ID NO: 100) |
| siRNA_b2_32 | UAAUUCCAGGCCAACAAAC | GUUUGUUGGCCUGGAAUUA |
| | (SEQ ID NO: 101) | (SEQ ID NO: 102) |
| siRNA_b2_33 | AUUCCAUUCAGCACUUUGC | GCAAAGUGCUGAAUGGAAU |
| | (SEQ ID NO: 103) | (SEQ ID NO: 104) |
| siRNA_b2_34 | UACCUGUUUAUUCAGUGGC | GCCACUGAAUAAACAGGUA |
| | (SEQ ID NO: 105) | (SEQ ID NO: 106) |
| siRNA_b2_35 | AAUUCAGUACUCUCUCUGG | CCAGAGAGAGUACUGAAUU |
| | (SEQ ID NO: 107) | (SEQ ID NO: 108) |
| siRNA_b2_36 | UAGUUCUUGGGAAUGAAGC | GCUUCAUUCCCAAGAACUA |
| | (SEQ ID NO: 109) | (SEQ ID NO: 110) |
| siRNA_b2_37 | UUUUGCCAAAAAACCACGG | CCGUGGUUUUUUGGCAAAA |
| | (SEQ ID NO: 111) | (SEQ ID NO: 112) |
| siRNA_b2_38 | AAACUUGACAGAGAGGGAG | CUCCCUCUCUGUCAAGUUU |
| | (SEQ ID NO: 113) | (SEQ ID NO: 114) |
| siRNA_b2_39 | AAUAUCUGCUGGUUUCUGG | CCAGAAACCAGCAGAUAUU |
| | (SEQ ID NO: 115) | (SEQ ID NO: 116) |
| siRNA_b2_40 | UGAGUUAUCCAUGACAUGG | CCAUGUCAUGGAUAACUCA |
| | (SEQ ID NO: 117) | (SEQ ID NO: 118) |
| siRNA_b2_41 | AAAGAAGGGUUGCACUUGC | GCAAGUGCAACCCUUCUUU |
| | (SEQ ID NO: 119) | (SEQ ID NO: 120) |
| siRNA_b2_42 | UAAGGAUCAACAAGGCUCC | GGAGCCUUGUUGAUCCUUA |
| | (SEQ ID NO: 121) | (SEQ ID NO: 122) |
| siRNA_b2_43 | UUUUGUUCCGAAGCCCAUG | CAUGGGCUUCGGAACAAAA |
| | (SEQ ID NO: 123) | (SEQ ID NO: 124) |
| siRNA_b2_44 | UAUCUGUGAAGGCAGAAGG | CCUUCUGCCUUCACAGAUA |
| | (SEQ ID NO: 125) | (SEQ ID NO: 126) |
| siRNA_b2_45 | UUAUGGGCGAAGUCCUUUG | CAAAGGACUUCGCCCAUAA |
| | (SEQ ID NO: 127) | (SEQ ID NO: 128) |
| siRNA_b2_46 | AAAUUCACCAGAAGGCAUC | GAUGCCUUCUGGUGAAUUU |
| | (SEQ ID NO: 129) | (SEQ ID NO: 130) |
| siRNA_b2_47 | UUUCCAAGUUCUCCACUUG | CAAGUGGAGAACUUGGAAA |
| | (SEQ ID NO: 131) | (SEQ ID NO: 132) |
| siRNA_b2_48 | UAUGGUAACAGCUUCCUCC | GGAGGAAGCUGUUACCAUA |
| | (SEQ ID NO: 133) | (SEQ ID NO: 134) |
| siRNA_b2_49 | AUACUGAGUGUCACCGUUG | CAACGGUGACACUCAGUAU |
| | (SEQ ID NO: 135) | (SEQ ID NO: 136) |
| siRNA_b2_50 | UCUUCAUCCUCGAUCUUGG | CCAAGAUCGAGGAUGAAGA |
| | (SEQ ID NO: 137) | (SEQ ID NO: 138) |
| siRNA_b2_51 | UGUUUCCUGCACAUGUUUG | CAAACAUGUGCAGGAAACA |
| | (SEQ ID NO: 139) | (SEQ ID NO: 140) |
| siRNA_b2_52 | UUCCACACCGAACUUGUUG | CAACAAGUUCGGUGUGGAA |
| | (SEQ ID NO: 141) | (SEQ ID NO: 142) |
| siRNA_b2_53 | UUAACGUGCUUCCAUUCCG | CGGAAUGGAAGCACGUUAA |
| | (SEQ ID NO: 143) | (SEQ ID NO: 144) |
| siRNA_b2_54 | UAGUAUGACCCUCGAUGAG | CUCAUCGAGGGUCAUACUA |
| | (SEQ ID NO: 145) | (SEQ ID NO: 146) |
| siRNA_b2_55 | UCAUAGUAGACAUUCACCC | GGGUGAAUGUCUACUAUGA |
| | (SEQ ID NO: 147) | (SEQ ID NO: 148) |
| siRNA_b2_56 | AGUAACUGGACAUCGAACC | GGUUCGAUGUCCAGUUACU |
| | (SEQ ID NO: 149) | (SEQ ID NO: 150) |
| siRNA_b2_57 | AGAAUGGUGAUGCGUUCAC | GUGAACGCAUCACCAUUCU |
| | (SEQ ID NO: 151) | (SEQ ID NO: 152) |
| siRNA_b2_58 | UGUAUCUAUAGAUGGCGAG | CUCGCCAUCUAUAGAUACA |
| | (SEQ ID NO: 153) | (SEQ ID NO: 154) |
| siRNA_b2_59 | UUUGGAGCACUGAAAAUCG | CGAUUUUCAGUGCUCCAAA |
| | (SEQ ID NO: 155) | (SEQ ID NO: 156) |
| siRNA_b2_60 | UAGAGUAUCGUCAAGUUCC | GGAACUUGACGAUACUCUA |
| | (SEQ ID NO: 157) | (SEQ ID NO: 158) |
| siRNA_b2_61 | UAAAGCGGCCAUUGUCUUG | CAAGACAAUGGCCGCUUUA |
| | (SEQ ID NO: 159) | (SEQ ID NO: 160) |
| siRNA_b2_62 | UGAAUCACAGUCUCUCCUG | CAGGAGAGACUGUGAUUCA |
| | (SEQ ID NO: 161) | (SEQ ID NO: 162) |
| siRNA_b2_63 | UUCUUCUAUAGCUGUCUCG | CGAGACAGCUAUAGAAGAA |
| | (SEQ ID NO: 163) | (SEQ ID NO: 164) |
| siRNA_b2_64 | UAAGACGUUCCCACUUGUC | GACAAGUGGGAACGUCUUA |
| | (SEQ ID NO: 165) | (SEQ ID NO: 166) |
| siRNA_b2_65 | AAAACUGUUGUACUGCUGG | CCAGCAGUACAACAGUUUU |
| | (SEQ ID NO: 167) | (SEQ ID NO: 168) |
| siRNA_b2_66 | UUACUUUGUGACUGUCCAC | GUGGACAGUCACAAAGUAA |
| | (SEQ ID NO: 169) | (SEQ ID NO: 170) |
| siRNA_b2_67 | UAUAAUCGCUCUUCACCUG | CAGGUGAAGAGCGAUUAUA |
| | (SEQ ID NO: 171) | (SEQ ID NO: 172) |
| siRNA_b2_68 | UUAGUGUUUUGGCCUUGAC | GUCAAGGCCAAAACACUAA |
| | (SEQ ID NO: 173) | (SEQ ID NO: 174) |
| siRNA_b2_69 | UUGGUAUUGAUGGCAAAGC | GCUUUGCCAUCAAUACCAA |
| | (SEQ ID NO: 175) | (SEQ ID NO: 176) |
| siRNA_b2_70 | AAUCAUUUGAGGACACCAG | CUGGUGUCCUCAAAUGAUU |
| | (SEQ ID NO: 177) | (SEQ ID NO: 178) |
| siRNA_b2_71 | UGUAAUACUGGACCAACUC | GAGUUGGUCCAGUAUUACATT |
| | (SEQ ID NO: 179) | (SEQ ID NO: 180) |
| siRNA_b2_72 | AAGAAUCAAACCGUUCUCC | GGAGAACGGUUUGAUUCUU |
| | (SEQ ID NO: 181) | (SEQ ID NO: 182) |
| siRNA_b2_73 | UGUAAUCUGAAACAGGCUC | GAGCCUGUUUCAGAUUACA |
| | (SEQ ID NO: 183) | (SEQ ID NO: 184) |
| siRNA_b2_74 | UUGUGUGGCAAUGUAACUC | GAGUUACAUUGCCACACAA |
| | (SEQ ID NO: 185) | (SEQ ID NO: 186) |
| siRNA_b2_75 | UUUCUUGGAACACCAUCCG | CGGAUGGUGUUCCAAGAAA |
| | (SEQ ID NO: 187) | (SEQ ID NO: 188) |
| siRNA_b2_76 | UUGUUCGGCAAGAAAACAC | GUGUUUUCUUGCCGAACAA |
| | (SEQ ID NO: 189) | (SEQ ID NO: 190) |
| siRNA_b2_77 | UUUCAUAAGGCAGUCAUGC | GCAUGACUGCCUUAUGAAA |
| | (SEQ ID NO: 191) | (SEQ ID NO: 192) |
| siRNA_b2_78 | UUUACCUUUGUGUUCGUGG | CCACGAACACAAAGGUAAA |
| | (SEQ ID NO: 193) | (SEQ ID NO: 194) |
| siRNA_b2_79 | UUGAGCAGGAAUUUCUGAC | GUCAGAAAUUCCUGCUCAA |
| | (SEQ ID NO: 195) | (SEQ ID NO: 196) |
| siRNA_b2_80 | UCUGAUGUUACUCCAGUCC | GGACUGGAGUAACAUCAGA |
| | (SEQ ID NO: 197) | (SEQ ID NO: 198) |
| siRNA_b2_81 | AAAGUUUGGCUGCUCUUUC | GAAAGAGCAGCCAAACUUU |
| | (SEQ ID NO: 199) | (SEQ ID NO: 200) |
| siRNA_b2_82 | AUUACUACUAUGCUGACCC | GGGUCAGCAUAGUAGUAAU |
| | (SEQ ID NO: 201) | (SEQ ID NO: 202) |
| siRNA_b2_83 | UUUACAUUGCCAAUCCCAC | GUGGGAUUGGCAAUGUAAA |
| | (SEQ ID NO: 203) | (SEQ ID NO: 204) |
| siRNA_b2_84 | ACUUAAAAGAGGCAGGAGC | GCUCCUGCCUCUUUUAAGU |
| | (SEQ ID NO: 205) | (SEQ ID NO: 206) |
| siRNA_b2_85 | UUUAGAGGCAUCACAAGCC | GGCUUGUGAUGCCUCUAAA |
| | (SEQ ID NO: 207) | (SEQ ID NO: 208) |
| siRNA_b2_86 | UUUAUAACCUAGGACCUCC | GGAGGUCCUAGGUUAUAAA |
| | (SEQ ID NO: 209) | (SEQ ID NO: 210) |
| siRNA_b2_87 | UAAGUUUGUUCUCCUGAGG | CCUCAGGAGAACAAACUUA |
| | (SEQ ID NO: 211) | (SEQ ID NO: 212) |
| siRNA_b2_88 | UAUUCUGCAUUGCUAGCAC | GUGCUAGCAAUGCAGAAUA |
| | (SEQ ID NO: 213) | (SEQ ID NO: 214) |
| siRNA_b2_89 | AUUUUCUUCUGGCGACUUG | CAAGUCGCCAGAAGAAAAU |
| | (SEQ ID NO: 215) | (SEQ ID NO: 216 |
| siRNA_b2_90 | UUCUGUUUCACUUUCAGGG | CCCUGAAAGUGAAACAGAA |
| | (SEQ ID NO: 217) | (SEQ ID NO: 218) |
| siRNA_b2_91 | UUAUAUUCGGCGUUUCGGG | CCCGAAACGCCGAAUAUAA |
| | (SEQ ID NO: 219) | (SEQ ID NO: 220) |
| siRNA_b2_92 | AAAAUCAGUGCCGUGGUUC | GAACCACGGCACUGAUUUU |
| | (SEQ ID NO: 221) | (SEQ ID NO: 222) |
| siRNA_b2_93 | AAAUUGUUGGUGGGUGAGC | GCUCACCCACCAACAAUUU |
| | (SEQ ID NO: 223) | (SEQ ID NO: 224) |
| siRNA_b2_94 | UCAACAUCCAUCUUCUCAC | GUGAGAAGAUGGAUGUUGA |
| | (SEQ ID NO: 225) | (SEQ ID NO: 226) |
| siRNA_b2_95 | AUAAAUAAAUGGGCAGCGC | GCGCUGCCCAUUUAUUUAU |
| | (SEQ ID NO: 227) | (SEQ ID NO: 228) |
| siRNA_b2_96 | AGCCUCUGUCCCAGUGCCC | GGGCACUGGGACAGAGGCU |
| | (SEQ ID NO: 229) | (SEQ ID NO: 230) |
| siRNA_b2_97 | UCAGCCUCUGUCCCAGUGC | GCACUGGGACAGAGGCUGA |
| | (SEQ ID NO: 231) | (SEQ ID NO: 232) |
| siRNA_b2_98 | UUUCUCAAACUCAGCCUCU | AGAGGCUGAGUUUGAGAAA |
| | (SEQ ID NO: 233) | (SEQ ID NO: 234) |
| siRNA_b2_99 | AGCUUUCUCAAACUCAGCC | GGCUGAGUUUGAGAAAGCU |
| | (SEQ ID NO: 235) | (SEQ ID NO: 236) |
| siRNA_b2_100 | UCCUCAUCCGAUGGCUUGG | CCAAGCCAUCGGAUGAGGA |
| | (SEQ ID NO: 237) | (SEQ ID NO: 238) |
| siRNA_b2_101 | UCAAUCUUGCUUGUUUGAC | GUCAAACAAGCAAGAUUGA |
| | (SEQ ID NO: 239) | (SEQ ID NO: 240) |
| siRNA_b2_102 | UCUCAAUCUUGCUUGUUUG | CAAACAAGCAAGAUUGAGA |
| | (SEQ ID NO: 241) | (SEQ ID NO: 242) |
| siRNA_b2_103 | UAAUCCAUGUCAGAUUCAG | CUGAAUCUGACAUGGAUUA |
| | (SEQ ID NO: 243) | (SEQ ID NO: 244) |
| siRNA_b2_104 | AAUUUCGGAAGGAAUAGAC | GUCUAUUCCUUCCGAAAUU |
| | (SEQ ID NO: 245) | (SEQ ID NO: 246) |
| siRNA_b2_105 | UUGAAUUUGCCUUUGAACC | GGUUCAAAGGCAAAUUCAA |
| | (SEQ ID NO: 247) | (SEQ ID NO: 248) |
| siRNA_b2_106 | UGAAAUCACAGCAUCGUUG | CAACGAUGCUGUGAUUUCA |
| | (SEQ ID NO: 249) | (SEQ ID NO: 250) |
| siRNA_b2_107 | AUUUACUCCAGAAAGGUUC | GAACCUUUCUGGAGUAAAU |
| | (SEQ ID NO: 251) | (SEQ ID NO: 252) |
| siRNA_b2_108 | UUACCAUAGCGUUUGUUUG | CAAACAAACGCUAUGGUAA |
| | (SEQ ID NO: 253) | (SEQ ID NO: 254) |
| siRNA_b2_109 | AUUUCUUCUGUCAUUGUCC | GGACAAUGACAGAAGAAAU |
| | (SEQ ID NO: 255) | (SEQ ID NO: 256) |
| siRNA_b2_110 | UAGAAUGUGGCGAUACAUC | GAUGUAUCGCCACAUUCUA |
| | (SEQ ID NO: 257) | (SEQ ID NO: 258) |
| siRNA_b2_111 | UGAAUCAUCCCAUUGUUCC | GGAACAAUGGGAUGAUUCA |
| | (SEQ ID NO: 259) | (SEQ ID NO: 260) |
| siRNA_b2_112 | UAUAACUGUGGCUUAACGC | GCGUUAAGCCACAGUUAUA |
| | (SEQ ID NO: 261) | (SEQ ID NO: 262) |
| siRNA_b2_113 | AUUCUGAUGCGAUGGUUUG | CAAACCAUCGCAUCAGAAU |
| | (SEQ ID NO: 263) | (SEQ ID NO: 264) |
| siRNA_b2_114 | AUUCUCAAGACUCGUAAUG | CAUUACGAGUCUUGAGAAU |
| | (SEQ ID NO: 265) | (SEQ ID NO: 266) |
| siRNA_b2_115 | UCAUAAACUGGCUUUAGAC | GUCUAAAGCCAGUUUAUGA |
| | (SEQ ID NO: 267) | (SEQ ID NO: 268) |
| siRNA_b2_116 | AAUGAUGUCCAAUGAGUUG | CAACUCAUUGGACAUCAUU |
| | (SEQ ID NO: 269) | (SEQ ID NO: 270) |
| siRNA_b2_117 | UGAAUUAGGGCACAUUGAG | CUCAAUGUGCCCUAAUUCA |
| | (SEQ ID NO: 271) | (SEQ ID NO: 272) |
| siRNA_b2_118 | UAUUAUUCGCCUCUUUCGG | CCGAAAGAGGCGAAUAAUA |
| | (SEQ ID NO: 273) | (SEQ ID NO: 274) |
| siRNA_b2_119 | UAUAGUUCAGCAGUUGAAG | CUUCAACUGCUGAACUAUA |
| | (SEQ ID NO: 275) | (SEQ ID NO: 276) |
| siRNA_b2_120 | UCACUAACCUGUAAUGUGC | GCACAUUACAGGUUAGUGA |
| | (SEQ ID NO: 277) | (SEQ ID NO: 278) |
| siRNA_b2_121 | UUAUGGAAGGCAAAGUCUC | GAGACUUUGCCUUCCAUAA |
| | (SEQ ID NO: 279) | (SEQ ID NO: 280) |
| siRNA_b2_122 | UGAUACAACUGUGAAAGAC | GUCUUUCACAGUUGUAUCA |
| | (SEQ ID NO: 281) | (SEQ ID NO: 282) |
| siRNA_b2_123 | AAAUUAGGGUUGCAUUUGG | CCAAAUGCAACCCUAAUUU |
| | (SEQ ID NO: 283) | (SEQ ID NO: 284) |
| siRNA_b2_124 | UAAACCAUCUUGAUUGUGC | GCACAAUCAAGAUGGUUUA |
| | (SEQ ID NO: 285) | (SEQ ID NO: 286) |
| siRNA_b2_125 | UUAUAACGCCUGUAACUCC | GGAGUUACAGGCGUUAUAA |
| | (SEQ ID NO: 287) | (SEQ ID NO: 288) |
| siRNA_b2_126 | AUUAUAACGCCUGUAACUC | GAGUUACAGGCGUUAUAAU |
| | (SEQ ID NO: 289) | (SEQ ID NO: 290) |
| siRNA_b2_127 | AGAAUAAAGCGAUAACUGC | GCAGUUAUCGCUUUAUUCU |
| | (SEQ ID NO: 291) | (SEQ ID NO: 292) |
| siRNA_b2_128 | AUUAGUAGGAGUAAUUCCC | GGGAAUUACUCCUACUAAU |
| | (SEQ ID NO: 293) | (SEQ ID NO: 294) |
| siRNA_b2_129 | ACUUUCACACGGUAACUGG | CCAGUUACCGUGUGAAAGU |
| | (SEQ ID NO: 295) | (SEQ ID NO: 296) |
| siRNA_b2_130 | AUUGUGAUCAAGUAGAAGG | CCUUCUACUUGAUCACAAU |
| | (SEQ ID NO: 297) | (SEQ ID NO: 298) |
| siRNA_b2_131 | UAUAUUAGGGCAAUCAUGC | GCAUGAUUGCCCUAAUAUA |
| | (SEQ ID NO: 299) | (SEQ ID NO: 300) |
| siRNA_b2_132 | UACAAGAAUCACUUUGUGC | GCACAAAGUGAUUCUUGUA |
| | (SEQ ID NO: 301) | (SEQ ID NO: 302) |
| siRNA_b2_133 | AAAGUGAUGUUCGUUGUAG | CUACAACGAACAUCACUUU |
| | (SEQ ID NO: 303) | (SEQ ID NO: 304) |
| siRNA_b2_134 | AUAUUGGAUCGAAUCAACG | CGUUGAUUCGAUCCAAUAU |
| | (SEQ ID NO: 305) | (SEQ ID NO: 306) |
| siRNA_b2_135 | UUGUUUGAGGGAUUCUGAG | CUCAGAAUCCCUCAAACAA |
| | (SEQ ID NO: 307) | (SEQ ID NO: 308) |
| siRNA_b2_136 | UUUACAGUUGCGUAGUUGC | GCAACUACGCAACUGUAAA |
| | (SEQ ID NO: 309) | (SEQ ID NO: 310) |
| siRNA_b2_137 | AUAUGUUUCGGGAGUUUAC | GUAAACUCCCGAAACAUAU |
| | (SEQ ID NO: 311) | (SEQ ID NO: 312) |
| siRNA_b2_138 | AAAUCUAUGGGCAAUGUCG | CGACAUUGCCCAUAGAUUU |
| | (SEQ ID NO: 313) | (SEQ ID NO: 314) |
| siRNA_b2_139 | UGAAAGUGUUCAUCAACAC | GUGUUGAUGAACACUUUCA |
| | (SEQ ID NO: 315) | (SEQ ID NO: 316) |
| siRNA_b2_140 | AUGUUGAUCUAGAGUUUCC | GGAAACUCUAGAUCAACAU |
| | (SEQ ID NO: 317) | (SEQ ID NO: 318) |
| siRNA_b2_141 | AGAAACAAUGUGUGUAUGC | GCAUACACACAUUGUUUCU |
| | (SEQ ID NO: 319) | (SEQ ID NO: 320) |
| siRNA_b2_142 | UUAGAGUUGUGUUGAAUCG | CGAUUCAACACAACUCUAA |
| | (SEQ ID NO: 321) | (SEQ ID NO: 322) |
| siRNA_b2_143 | UCACUAUAGGGCGUAAUGC | GCAUUACGCCCUAUAGUGA |
| | (SEQ ID NO: 323) | (SEQ ID NO: 324) |
| siRNA_b2_144 | AUAUCCUAGAACAUUAGGC | GCCUAAUGUUCUAGGAUAU |
| | (SEQ ID NO: 325) | (SEQ ID NO: 326) |
| siRNA_b2_145 | UCAUAUUGCUAGGAAAUGC | GCAUUUCCUAGCAAUAUGA |
| | (SEQ ID NO: 327) | (SEQ ID NO: 328) |
| siRNA_b2_146 | UAUUGAACCCGAGAUGAUG | CAUCAUCUCGGGUUCAAUA |
| | (SEQ ID NO: 329) | (SEQ ID NO: 330) |
| siRNA_b2_147 | AUAUUGUUCCGAAAUCCAG | CUGGAUUUCGGAACAAUAU |
| | (SEQ ID NO: 331) | (SEQ ID NO: 332) |
| siRNA_b2_148 | UGAUAUUGUUCCGAAAUCC | GGAUUUCGGAACAAUAUCA |
| | (SEQ ID NO: 333) | (SEQ ID NO: 334) |
| siRNA_b2_149 | UUGUUGAUGAUCUUAGAGG | CCUCUAAGAUCAUCAACAA |
| | (SEQ ID NO: 335) | (SEQ ID NO: 336) |
| siRNA_b2_150 | UAUCUUCUGUCCUUGAUCC | GGAUCAAGGACAGAAGAUA |
| | (SEQ ID NO: 337) | (SEQ ID NO: 338) |
| siRNA_b2_151 | GCCAGAAUGCUACGGAGAU | AUCUCCGUAGCAUUCUGGC |
| | (SEQ ID NO: 339) | (SEQ ID NO: 340) |
| siRNA_b2_152 | GCUGAUUCAGAACAGUAUA | UAUACUGUUCUGAAUCAGC |
| | (SEQ ID NO: 341) | (SEQ ID NO: 342) |
| siRNA_b2_153 | GCCUUACUCAUCUGAUGAU | AUCAUCAGAUGAGUAAGGC |
| | (SEQ ID NO: 343) | (SEQ ID NO: 344) |
| siRNA_b2_154 | CCUGAAUGAUGCCACAUAU | AUAUGUGGCAUCAUUCAGG |
| | (SEQ ID NO: 345) | (SEQ ID NO: 346) |
| siRNA_b2_155 | GAGAAGGGUACUCCCUGGU | ACCAGGGAGUACCCUUCUC |
| | (SEQ ID NO: 347) | (SEQ ID NO: 348) |
| siRNA_b2_156 | GCAGAGGAGUAUGACAAUU | AAUUGUCAUACUCCUCUGC |
| | (SEQ ID NO: 349) | (SEQ ID NO: 350) |
| siRNA_b2_157 | GCACUUACAUUGAACACAA | UUGUGUUCAAUGUAAGUGC |
| | (SEQ ID NO: 351) | (SEQ ID NO: 352) |
| siRNA_b2_158 | GGAUGUUUCUAGCAAUGAU | AUCAUUGCUAGAAACAUCC |
| | (SEQ ID NO: 353) | (SEQ ID NO: 354) |
| siRNA_b2_159 | GCGGAAAUGCUCGCAAAUA | UAUUUGCGAGCAUUUCCGC |
| | (SEQ ID NO: 355) | (SEQ ID NO: 356) |
| siRNA_b2_160 | GGUUCUAUAGAACCUGCAA | UUGCAGGUUCUAUAGAACC |
| | (SEQ ID NO: 357) | (SEQ ID NO: 358) |
| siRNA_b2_161 | GGAGUUUCAAUUCUGAAUC | GAUUCAGAAUUGAAACUCC |
| | (SEQ ID NO: 359) | (SEQ ID NO: 360) |
| siRNA_b2_162 | GACUCCAAUCCUCAGAUGA | UCAUCUGAGGAUUGGAGUC |
| | (SEQ ID NO: 361) | (SEQ ID NO: 362) |
| siRNA_b2_163 | GGAGAAGAAUCCUGCCCUU | AAGGGCAGGAUUCUUCUCC |
| | (SEQ ID NO: 363) | (SEQ ID NO: 364) |
| siRNA_b2_164 | GGUGUUGCAUUUGACCCAA | UUGGGUCAAAUGCAACACC |
| | (SEQ ID NO: 365) | (SEQ ID NO: 366) |
| siRNA_b2_165 | GCGAAGAGCAACAGCCAUU | AAUGGCUGUUGCUCUUCGC |
| | (SEQ ID NO: 367) | (SEQ ID NO: 368) |
| siRNA_b2_166 | GGGAAAGACGAGCAAUCAA | UUGAUUGCUCGUCUUUCCC |
| | (SEQ ID NO: 369) | (SEQ ID NO: 370) |
| siRNA_b2_167 | GCAUCAACUCCUGAGGCAU | AUGCCUCAGGAGUUGAUGCTT |
| | (SEQ ID NO: 371) | (SEQ ID NO: 372) |
| siRNA_b2_168 | GGAGUAGAUGAAUAUUCCA | UGGAAUAUUCAUCUACUCC |
| | (SEQ ID NO: 373) | (SEQ ID NO: 374) |
| siRNA_b2_169 | GCUCUCAGCGGUCGAAAUU | AAUUUCGACCGCUGAGAGC |
| | (SEQ ID NO: 375) | (SEQ ID NO: 376) |
| siRNA_b2_170 | GCAGGUGCUAGCAGAACUU | AAGUUCUGCUAGCACCUGC |
| | (SEQ ID NO: 377) | (SEQ ID NO: 378) |
| siRNA_b2_171 | GCAGGGCUACUGAAUAUAU | AUAUAUUCAGUAGCCCUGC |
| | (SEQ ID NO: 379) | (SEQ ID NO: 380) |
| siRNA_b2_172 | GCAGUAGGCCAAGUGUCAA | UUGACACUUGGCCUACUGC |
| | (SEQ ID NO: 381) | (SEQ ID NO: 382) |
| siRNA_b2_173 | CAACUCCUUCCUCACACAU | AUGUGUGAGGAAGGAGUUG |
| | (SEQ ID NO: 383) | (SEQ ID NO: 384) |
| siRNA_b2_174 | AGGGAGUGUACAUAAAUAC | GUAUUUAUGUACACUCCCU |
| | (SEQ ID NO: 385) | (SEQ ID NO: 386) |
| siRNA_b2_175 | GCUCUCAUGGAGUGGAUAA | UUAUCCACUCCAUGAGAGC |
| | (SEQ ID NO: 387) | (SEQ ID NO: 388) |
| siRNA_b2_176 | GGACUAGUAUGUGCCACUU | AAGUGGCACAUACUAGUCC |
| | (SEQ ID NO: 389) | (SEQ ID NO: 390) |
| siRNA_b2_177 | GCACUACGGCUAAGGCUAU | AUAGCCUUAGCCGUAGUGC |
| | (SEQ ID NO: 391) | (SEQ ID NO: 392) |
| siRNA_b2_178 | GGAAGAAUAUCGGCAGGAA | UUCCUGCCGAUAUUCUUCC |
| | (SEQ ID NO: 393) | (SEQ ID NO: 394) |
| siRNA_b2_179 | GGAGUGGAUAAAGACAAGA | UCUUGUCUUUAUCCACACC |
| | (SEQ ID NO: 395) | (SEQ ID NO: 396) |
| siRNA_b2_180 | CUAACUCCAGUACAGGUCU | AGACCUGUACUGGAGUUAG |
| | (SEQ ID NO: 397) | (SEQ ID NO: 398) |

B. Obtaining Input and Output Values for Establishing a Machine Learning Model

Among them, the input values of any one of the 180 siRNAs were obtained as follows:

i) aligning siRNA sequences with human genomic mRNA sequences, and further screening off-target genes based on functional annotation and expression profile database

In order to preliminarily determine the off-target gene of a certain siRNA, a localized mRNA sequence database of the human genome (that is, downloading the mRNA sequences to a hard disk, such that subsequent work can be done independently of the network) was established by BLAST (version number 2.2.31) software (see the literature: “Camacho C, Coulouris G, Avagyan V, Ma N, Papadopoulos J, Bealer K, Madden TL. BLAST+: architecture and applications. BMC Bioinformatics. 2008, 10:421.”). The sequence of the siRNA and the mRNA sequence data of the human genome were comprehensively aligned. In order to obtain comprehensive alignment results, but not just highly similar alignment, in the BLAST software the blastn mode was chosen. Most of the parameter settings of the BLAST software adopted the default parameters, as follows: evalue=1000, word_size=7, gapopen=5, gapextend=2, penalty=3, reward=2. During alignment, the sense and antisense strands of the siRNA were aligned, respectively.

By alignment, a complete preliminary off-target gene list was obtained, and then the region where the siRNA and each off-target gene's mRNA match was functionally annotated as to whether the action region of the siRNA was distributed in the 5′ UTR, 3′ UTR or coding region of the mRNA. Based on the principle of action of siRNA, only such an off-target gene that the siRNA matching site was located in the 3′ UTR and/or coding region of its mRNA was concerned in the subsequent analysis.

The off-target gene that was not expressed by itself in human respiratory cells (for example, non-small cell lung cancer cell line A549) was deleted from the off-target gene list, using the expression profile database of the known cell line. The expression profile data for the cell line was derived from the “THE HUMAN PROTEIN ATLAS” database.

A series of off-target genes were thus selected. For each of the 180 siRNAs, hundreds of off-target genes were obtained. The specific statistical results of the number of off-target genes are shown in Table 11.

TABLE 11
Statistics on the number of off-target genes of siRNAs

| siRNA name | Number of off-target genes |
|---|---|
| siRNA_b2_1 | 156 |
| siRNA_b2_2 | 216 |
| siRNA_b2_3 | 103 |
| siRNA_b2_4 | 173 |
| siRNA_b2_5 | 220 |
| siRNA_b2_6 | 635 |
| siRNA_b2_7 | 366 |
| siRNA_b2_8 | 162 |
| siRNA_b2_9 | 150 |
| siRNA_b2_10 | 315 |
| siRNA_b2_11 | 271 |
| siRNA_b2_12 | 43 |
| siRNA_b2_13 | 310 |
| siRNA_b2_14 | 193 |
| siRNA_b2_15 | 205 |
| siRNA_b2_16 | 161 |
| siRNA_b2_17 | 219 |
| siRNA_b2_18 | 257 |
| siRNA_b2_19 | 169 |
| siRNA_b2_20 | 152 |
| siRNA_b2_21 | 120 |
| siRNA_b2_22 | 269 |
| siRNA_b2_23 | 126 |
| siRNA_b2_24 | 108 |
| siRNA_b2_25 | 193 |
| siRNA_b2_26 | 98 |
| siRNA_b2_27 | 31 |
| siRNA_b2_28 | 292 |
| siRNA_b2_29 | 357 |
| siRNA_b2_30 | 125 |
| siRNA_b2_31 | 367 |
| siRNA_b2_32 | 225 |
| siRNA_b2_33 | 307 |
| siRNA_b2_34 | 283 |
| siRNA_b2_35 | 164 |
| siRNA_b2_36 | 280 |
| siRNA_b2_37 | 272 |
| siRNA_b2_38 | 246 |
| siRNA_b2_39 | 307 |
| siRNA_b2_40 | 146 |
| siRNA_b2_41 | 111 |
| siRNA_b2_42 | 131 |
| siRNA_b2_43 | 102 |
| siRNA_b2_44 | 358 |
| siRNA_b2_45 | 76 |
| siRNA_b2_46 | 273 |
| siRNA_b2_47 | 370 |
| siRNA_b2_48 | 216 |
| siRNA_b2_49 | 83 |
| siRNA_b2_50 | 183 |
| siRNA_b2_51 | 271 |
| siRNA_b2_52 | 70 |
| siRNA_b2_53 | 91 |
| siRNA_b2_54 | 52 |
| siRNA_b2_55 | 132 |
| siRNA_b2_56 | 81 |
| siRNA_b2_57 | 99 |
| siRNA_b2_58 | 59 |
| siRNA_b2_59 | 271 |
| siRNA_b2_60 | 47 |
| siRNA_b2_61 | 110 |
| siRNA_b2_62 | 286 |
| siRNA_b2_63 | 143 |
| siRNA_b2_64 | 74 |
| siRNA_b2_65 | 214 |
| siRNA_b2_66 | 217 |
| siRNA_b2_67 | 112 |
| siRNA_b2_68 | 211 |
| siRNA_b2_69 | 222 |
| siRNA_b2_70 | 265 |
| siRNA_b2_71 | 132 |
| siRNA_b2_72 | 86 |
| siRNA_b2_73 | 232 |
| siRNA_b2_74 | 150 |
| siRNA_b2_75 | 213 |
| siRNA_b2_76 | 118 |
| siRNA_b2_77 | 155 |
| siRNA_b2_78 | 173 |
| siRNA_b2_79 | 316 |
| siRNA_b2_80 | 157 |
| siRNA_b2_81 | 281 |
| siRNA_b2_82 | 111 |
| siRNA_b2_83 | 156 |
| siRNA_b2_84 | 394 |
| siRNA_b2_85 | 176 |
| siRNA_b2_86 | 58 |
| siRNA_b2_87 | 317 |
| siRNA_b2_88 | 146 |
| siRNA_b2_89 | 179 |
| siRNA_b2_90 | 477 |
| siRNA_b2_91 | 20 |
| siRNA_b2_92 | 107 |
| siRNA_b2_93 | 217 |
| siRNA_b2_94 | 405 |
| siRNA_b2_95 | 201 |
| siRNA_b2_96 | 373 |
| siRNA_b2_97 | 429 |
| siRNA_b2_98 | 347 |
| siRNA_b2_99 | 388 |
| siRNA_b2_100 | 87 |
| siRNA_b2_101 | 225 |
| siRNA_b2_102 | 203 |
| siRNA_b2_103 | 167 |
| siRNA_b2_104 | 110 |
| siRNA_b2_105 | 314 |
| siRNA_b2_106 | 188 |
| siRNA_b2_107 | 297 |
| siRNA_b2_108 | 54 |
| siRNA_b2_109 | 397 |
| siRNA_b2_110 | 75 |
| siRNA_b2_111 | 152 |
| siRNA_b2_112 | 78 |
| siRNA_b2_113 | 65 |
| siRNA_b2_114 | 57 |
| siRNA_b2_115 | 214 |
| siRNA_b2_116 | 177 |
| siRNA_b2_117 | 91 |
| siRNA_b2_118 | 77 |
| siRNA_b2_119 | 222 |
| siRNA_b2_120 | 93 |
| siRNA_b2_121 | 317 |
| siRNA_b2_122 | 250 |
| siRNA_b2_123 | 139 |
| siRNA_b2_124 | 185 |
| siRNA_b2_125 | 78 |
| siRNA_b2_126 | 65 |
| siRNA_b2_127 | 52 |
| siRNA_b2_128 | 84 |
| siRNA_b2_129 | 60 |
| siRNA_b2_130 | 159 |
| siRNA_b2_131 | 76 |
| siRNA_b2_132 | 269 |
| siRNA_b2_133 | 73 |
| siRNA_b2_134 | 41 |
| siRNA_b2_135 | 193 |
| siRNA_b2_136 | 27 |
| siRNA_b2_137 | 67 |
| siRNA_b2_138 | 115 |
| siRNA_b2_139 | 264 |
| siRNA_b2_140 | 115 |
| siRNA_b2_141 | 216 |
| siRNA_b2_142 | 119 |
| siRNA_b2_143 | 21 |
| siRNA_b2_144 | 120 |
| siRNA_b2_145 | 126 |
| siRNA_b2_146 | 63 |
| siRNA_b2_147 | 75 |
| siRNA_b2_148 | 61 |
| siRNA_b2_149 | 197 |
| siRNA_b2_150 | 310 |
| siRNA_b2_151 | 66 |
| siRNA_b2_152 | 279 |
| siRNA_b2_153 | 192 |
| siRNA_b2_154 | 219 |
| siRNA_b2_155 | 100 |
| siRNA_b2_156 | 210 |
| siRNA_b2_157 | 147 |
| siRNA_b2_158 | 181 |
| siRNA_b2_159 | 56 |
| siRNA_b2_160 | 96 |
| siRNA_b2_161 | 286 |
| siRNA_b2_162 | 159 |
| siRNA_b2_163 | 266 |
| siRNA_b2_164 | 154 |
| siRNA_b2_165 | 273 |
| siRNA_b2_166 | 74 |
| siRNA_b2_167 | 262 |
| siRNA_b2_168 | 162 |
| siRNA_b2_169 | 25 |
| siRNA_b2_170 | 157 |
| siRNA_b2_171 | 132 |
| siRNA_b2_172 | 104 |
| siRNA_b2_173 | 404 |
| siRNA_b2_174 | 154 |
| siRNA_b2_175 | 185 |
| siRNA_b2_176 | 85 |
| siRNA_b2_177 | 33 |
| siRNA_b2_178 | 97 |
| siRNA_b2_179 | 230 |
| siRNA_b2_180 | 136 |

ii) Determining the off-target weights of the selected off-target genes

The interference rate of the curve fitting obtained in Experimental Example 1 was used as a standard, and weights were set for the respective off-target genes.

For example, if the matched region of a specific off-target gene, human ERCC6 (Excision Repair Cross-Complementation 6), with a specific siRNA, e.g., siRNA4 (sense strand sequence CCAGACAGGUGUUAUGGAATT (SEQ ID NO: 7)), has 1 mismatch at the 3′ end of the sense strand and 5 mismatches at the 5′ end of the sense strand, then the overall interference rate of the siRNA on the off-target gene is the product of interference rates at both ends, i.e., 0.9782 timed 0.7880 is equal to 0.7708.

For the complementary region, the software RNAPLFOLD (version 2.2.4) (see the literature: “Lewis B P, Burge C B, Bartel D P. Conserved seed pairing, often flanked by adenosines, indicates that thousands of human genes are microRNA targets. Cell. 2005, 120(1): 15-20.”) was used to determine the probability of the off-target gene's mRNA itself not forming a secondary structure. Specifically, the software was used to predict the secondary structure of human whole genome mRNA and extract the relevant text and numerical information from the output to form a localized database for high-speed reading and improvement of calculation speed. The parameter design of RNAPLFOLD includes: L=40, W=80, u=25. For example, in the region where the mRNA of the off-target gene was complementary to the siRNA sequence, the probability of the off-target gene not forming a secondary structure was 0.5425, and the overall interference rate, obtained based on the interference rates at both ends, was 0.7708, such that the off-target weight of the off-target gene was 0.5425×0.7708=0.4182.

iii)-vi) obtaining omic weights based on omic annotation of the selected off-target genes; and calculating omic eigenvalues from the omic weights and off-target weights

(1) Calculating Proteomic Eigenvalues Based on the Protein Interaction Weights and Off-Target Weights of all the Selected Off-Target Genes

The human LINKS table in the STRING database was localized (that is, downloaded to the hard disk of a local computer), and the names of proteins were converted into common gene names for calculation operations. Cells were treated with a specific siRNA, and the possible off-target genes and their weights were determined by the methods described above. FIG. 6 illustratively shows a simplified example of a certain siRNA having seven off-target genes (represented by circles), each of which has an exemplary off-target weight (the number in the circle). Based on the information in the STRING database, those genes having interactions and the protein interaction weights thereof were determined. FIG. 6 exemplarily shows an example in which there are interactions among three genes that are connected by lines, with the numbers on the lines showing the weights of the interaction. Thus, the proteomic eigenvalue was calculated as follows: proteomic eigenvalue=0.9×(280+160)+0.8×280+0.6×160. That is, for each off-target gene, if it participates in an interaction, its off-target weight is multiplied by the protein interaction weight; if it participates in multiple interactions, its off-target weight is multiplied by the sum of the respective protein interaction weights; if the off-target gene is isolated, its effect is ignored. The results of calculation of proteomic eigenvalues of the off-target genes of the respective siRNAs are shown in Table 12.

TABLE 12
Calculation results of proteomic eigenvalues
of off-target genes of siRNAs

| siRNA name | Proteomic eigenvalue |
|---|---|
| siRNA_b2_1 | 175.5887 |
| siRNA_b2_2 | 321.0164 |
| siRNA_b2_3 | 51.6283 |
| siRNA_b2_4 | 276.2194 |
| siRNA_b2_5 | 407.8385 |
| siRNA_b2_6 | 3289.9944 |
| siRNA_b2_7 | 1056.2965 |
| siRNA_b2_8 | 179.5769 |
| siRNA_b2_9 | 98.1833 |
| siRNA_b2_10 | 574.1895 |
| siRNA_b2_11 | 379.3374 |
| siRNA_b2_12 | 21.9041 |
| siRNA_b2_13 | 896.0896 |
| siRNA_b2_14 | 253.1492 |
| siRNA_b2_15 | 337.5291 |
| siRNA_b2_16 | 192.7000 |
| siRNA_b2_17 | 336.4984 |
| siRNA_b2_18 | 499.3138 |
| siRNA_b2_19 | 255.5070 |
| siRNA_b2_20 | 188.5599 |
| siRNA_b2_21 | 129.1371 |
| siRNA_b2_22 | 568.5941 |
| siRNA_b2_23 | 150.8121 |
| siRNA_b2_24 | 87.7340 |
| siRNA_b2_25 | 193.3580 |
| siRNA_b2_26 | 73.2463 |
| siRNA_b2_27 | 8.2256 |
| siRNA_b2_28 | 503.0079 |
| siRNA_b2_29 | 762.3794 |
| siRNA_b2_30 | 117.0764 |
| siRNA_b2_31 | 966.1627 |
| siRNA_b2_32 | 264.5283 |
| siRNA_b2_33 | 561.0375 |
| siRNA_b2_34 | 549.5241 |
| siRNA_b2_35 | 242.2719 |
| siRNA_b2_36 | 610.1818 |
| siRNA_b2_37 | 695.6369 |
| siRNA_b2_38 | 317.6930 |
| siRNA_b2_39 | 666.4679 |
| siRNA_b2_40 | 195.9740 |
| siRNA_b2_41 | 121.8773 |
| siRNA_b2_42 | 126.4742 |
| siRNA_b2_43 | 72.4057 |
| siRNA_b2_44 | 820.6616 |
| siRNA_b2_45 | 22.4243 |
| siRNA_b2_46 | 446.0596 |
| siRNA_b2_47 | 892.2427 |
| siRNA_b2_48 | 362.8043 |
| siRNA_b2_49 | 38.4520 |
| siRNA_b2_50 | 454.7835 |
| siRNA_b2_51 | 367.9656 |
| siRNA_b2_52 | 29.8225 |
| siRNA_b2_53 | 52.7708 |
| siRNA_b2_54 | 27.9961 |
| siRNA_b2_55 | 111.9463 |
| siRNA_b2_56 | 80.4676 |
| siRNA_b2_57 | 78.4298 |
| siRNA_b2_58 | 26.5934 |
| siRNA_b2_59 | 433.9341 |
| siRNA_b2_60 | 38.5980 |
| siRNA_b2_61 | 113.3335 |
| siRNA_b2_62 | 531.1094 |
| siRNA_b2_63 | 161.4363 |
| siRNA_b2_64 | 42.4727 |
| siRNA_b2_65 | 374.4091 |
| siRNA_b2_66 | 305.4954 |
| siRNA_b2_67 | 103.9040 |
| siRNA_b2_68 | 341.6267 |
| siRNA_b2_69 | 487.1351 |
| siRNA_b2_70 | 438.4692 |
| siRNA_b2_71 | 109.9296 |
| siRNA_b2_72 | 61.5458 |
| siRNA_b2_73 | 345.6349 |
| siRNA_b2_74 | 116.8233 |
| siRNA_b2_75 | 330.4105 |
| siRNA_b2_76 | 108.5917 |
| siRNA_b2_77 | 110.3793 |
| siRNA_b2_78 | 181.9659 |
| siRNA_b2_79 | 662.6912 |
| siRNA_b2_80 | 194.2331 |
| siRNA_b2_81 | 452.0449 |
| siRNA_b2_82 | 112.2582 |
| siRNA_b2_83 | 220.5832 |
| siRNA_b2_84 | 873.0626 |
| siRNA_b2_85 | 154.6988 |
| siRNA_b2_86 | 21.2524 |
| siRNA_b2_87 | 822.1400 |
| siRNA_b2_88 | 146.0774 |
| siRNA_b2_89 | 209.7736 |
| siRNA_b2_90 | 1729.7501 |
| siRNA_b2_91 | 6.4001 |
| siRNA_b2_92 | 82.4334 |
| siRNA_b2_93 | 367.7844 |
| siRNA_b2_94 | 1579.4935 |
| siRNA_b2_95 | 292.6193 |
| siRNA_b2_96 | 1033.7495 |
| siRNA_b2_97 | 1329.0463 |
| siRNA_b2_98 | 837.8304 |
| siRNA_b2_99 | 1037.4298 |
| siRNA_b2_100 | 38.6853 |
| siRNA_b2_101 | 510.8298 |
| siRNA_b2_102 | 405.1226 |
| siRNA_b2_103 | 205.4264 |
| siRNA_b2_104 | 73.6244 |
| siRNA_b2_105 | 698.0153 |
| siRNA_b2_106 | 283.6909 |
| siRNA_b2_107 | 477.5030 |
| siRNA_b2_108 | 20.8498 |
| siRNA_b2_109 | 1186.9354 |
| siRNA_b2_110 | 31.1868 |
| siRNA_b2_111 | 133.5328 |
| siRNA_b2_112 | 26.1322 |
| siRNA_b2_113 | 38.1404 |
| siRNA_b2_114 | 23.6209 |
| siRNA_b2_115 | 431.0983 |
| siRNA_b2_116 | 300.4721 |
| siRNA_b2_117 | 53.7620 |
| siRNA_b2_118 | 57.4256 |
| siRNA_b2_119 | 298.4330 |
| siRNA_b2_120 | 26.0209 |
| siRNA_b2_121 | 582.7406 |
| siRNA_b2_122 | 527.2167 |
| siRNA_b2_123 | 155.8656 |
| siRNA_b2_124 | 224.1450 |
| siRNA_b2_125 | 38.8873 |
| siRNA_b2_126 | 48.8930 |
| siRNA_b2_127 | 13.7740 |
| siRNA_b2_128 | 55.0052 |
| siRNA_b2_129 | 15.5937 |
| siRNA_b2_130 | 288.2561 |
| siRNA_b2_131 | 31.2892 |
| siRNA_b2_132 | 310.9737 |
| siRNA_b2_133 | 31.8999 |
| siRNA_b2_134 | 13.6192 |
| siRNA_b2_135 | 254.1458 |
| siRNA_b2_136 | 8.2666 |
| siRNA_b2_137 | 29.7246 |
| siRNA_b2_138 | 69.2110 |
| siRNA_b2_139 | 385.2521 |
| siRNA_b2_140 | 79.0030 |
| siRNA_b2_141 | 285.3561 |
| siRNA_b2_142 | 123.6581 |
| siRNA_b2_143 | 2.1368 |
| siRNA_b2_144 | 90.1713 |
| siRNA_b2_145 | 66.2560 |
| siRNA_b2_146 | 19.6399 |
| siRNA_b2_147 | 26.7247 |
| siRNA_b2_148 | 26.9580 |
| siRNA_b2_149 | 167.7961 |
| siRNA_b2_150 | 733.3309 |
| siRNA_b2_151 | 23.4647 |
| siRNA_b2_152 | 572.3678 |
| siRNA_b2_153 | 176.2201 |
| siRNA_b2_154 | 227.4718 |
| siRNA_b2_155 | 36.4215 |
| siRNA_b2_156 | 342.8953 |
| siRNA_b2_157 | 174.2424 |
| siRNA_b2_158 | 193.8570 |
| siRNA_b2_159 | 11.6380 |
| siRNA_b2_160 | 43.4791 |
| siRNA_b2_161 | 583.5688 |
| siRNA_b2_162 | 144.8794 |
| siRNA_b2_163 | 406.3685 |
| siRNA_b2_164 | 183.2606 |
| siRNA_b2_165 | 498.1787 |
| siRNA_b2_166 | 41.6769 |
| siRNA_b2_167 | 372.0305 |
| siRNA_b2_168 | 240.7660 |
| siRNA_b2_169 | 6.8420 |
| siRNA_b2_170 | 159.0141 |
| siRNA_b2_171 | 79.5501 |
| siRNA_b2_172 | 64.6843 |
| siRNA_b2_173 | 1269.9319 |
| siRNA_b2_174 | 138.9317 |
| siRNA_b2_175 | 222.3294 |
| siRNA_b2_176 | 34.2568 |
| siRNA_b2_177 | 8.4215 |
| siRNA_b2_178 | 87.2334 |
| siRNA_b2_179 | 342.5200 |
| siRNA_b2_180 | 85.7611 |

(2) Calculating Signal Pathwayomic Eigenvalues Based on Signal Pathway Weights and Off-Target Weights of all the Selected Off-Target Genes

The human pathway database ConsensusPathDB-human (version number 31) was localized. By multiplying the determined off-target weight of each off-target gene and the number of pathways involved, and then calculating the sum, the signal pathwayomic eigenvalue was obtained. If the off-target gene was isolated, its effect was ignored. For example, three off-target genes A, B, and C were identified. According to the database, A was involved in 3 known pathways, B was involved in 2 known pathways, and C was isolated. Then, their signal pathwayomic eigenvalue was calculated as follows: (the off-target weight of A multiplied by 3) plus (the off-target weight of B multiplied by 2). The calculation results of the signal pathwayomic eigenvalues of the off-target genes of the respective siRNAs are shown in Table 13.

TABLE 13
Calculation results of signal pathwayomic
eigenvalues of off-target genes of siRNAs

| siRNA name | Signal pathwayomic eigenvalue |
|---|---|
| siRNA_b2_1 | 555.4674 |
| siRNA_b2_2 | 773.6962 |
| siRNA_b2_3 | 325.1144 |
| siRNA_b2_4 | 835.8499 |
| siRNA_b2_5 | 685.8466 |
| siRNA_b2_6 | 3844.7439 |
| siRNA_b2_7 | 1434.8959 |
| siRNA_b2_8 | 507.3778 |
| siRNA_b2_9 | 512.8768 |
| siRNA_b2_10 | 1153.7760 |
| siRNA_b2_11 | 830.6612 |
| siRNA_b2_12 | 253.1262 |
| siRNA_b2_13 | 1736.1900 |
| siRNA_b2_14 | 701.2423 |
| siRNA_b2_15 | 768.4803 |
| siRNA_b2_16 | 741.8769 |
| siRNA_b2_17 | 779.3116 |
| siRNA_b2_18 | 1458.9198 |
| siRNA_b2_19 | 808.6327 |
| siRNA_b2_20 | 637.2207 |
| siRNA_b2_21 | 689.9126 |
| siRNA_b2_22 | 1409.2702 |
| siRNA_b2_23 | 599.3609 |
| siRNA_b2_24 | 345.6021 |
| siRNA_b2_25 | 552.8839 |
| siRNA_b2_26 | 412.1261 |
| siRNA_b2_27 | 256.2023 |
| siRNA_b2_28 | 974.8122 |
| siRNA_b2_29 | 1079.8441 |
| siRNA_b2_30 | 528.5595 |
| siRNA_b2_31 | 1199.7935 |
| siRNA_b2_32 | 740.5331 |
| siRNA_b2_33 | 1001.1549 |
| siRNA_b2_34 | 1151.0407 |
| siRNA_b2_35 | 985.2212 |
| siRNA_b2_36 | 1447.5522 |
| siRNA_b2_37 | 1698.7274 |
| siRNA_b2_38 | 1466.0324 |
| siRNA_b2_39 | 1388.7937 |
| siRNA_b2_40 | 652.4307 |
| siRNA_b2_41 | 827.1734 |
| siRNA_b2_42 | 602.3023 |
| siRNA_b2_43 | 488.7201 |
| siRNA_b2_44 | 1438.0614 |
| siRNA_b2_45 | 327.1525 |
| siRNA_b2_46 | 732.9698 |
| siRNA_b2_47 | 1865.2309 |
| siRNA_b2_48 | 773.2206 |
| siRNA_b2_49 | 123.3686 |
| siRNA_b2_50 | 1259.1094 |
| siRNA_b2_51 | 917.8688 |
| siRNA_b2_52 | 284.2059 |
| siRNA_b2_53 | 465.5981 |
| siRNA_b2_54 | 283.2909 |
| siRNA_b2_55 | 357.9118 |
| siRNA_b2_56 | 740.2323 |
| siRNA_b2_57 | 350.6548 |
| siRNA_b2_58 | 276.4135 |
| siRNA_b2_59 | 900.4722 |
| siRNA_b2_60 | 485.4475 |
| siRNA_b2_61 | 424.6945 |
| siRNA_b2_62 | 1131.6280 |
| siRNA_b2_63 | 891.2678 |
| siRNA_b2_64 | 327.6220 |
| siRNA_b2_65 | 929.5330 |
| siRNA_b2_66 | 797.6966 |
| siRNA_b2_67 | 435.9446 |
| siRNA_b2_68 | 892.4617 |
| siRNA_b2_69 | 1302.5718 |
| siRNA_b2_70 | 919.2225 |
| siRNA_b2_71 | 503.8014 |
| siRNA_b2_72 | 332.7904 |
| siRNA_b2_73 | 1293.8742 |
| siRNA_b2_74 | 806.7452 |
| siRNA_b2_75 | 1641.1826 |
| siRNA_b2_76 | 614.5103 |
| siRNA_b2_77 | 548.7087 |
| siRNA_b2_78 | 830.5986 |
| siRNA_b2_79 | 1265.4671 |
| siRNA_b2_80 | 629.4345 |
| siRNA_b2_81 | 1017.5456 |
| siRNA_b2_82 | 596.5543 |
| siRNA_b2_83 | 655.9227 |
| siRNA_b2_84 | 1470.9432 |
| siRNA_b2_85 | 600.5681 |
| siRNA_b2_86 | 339.2501 |
| siRNA_b2_87 | 1337.8793 |
| siRNA_b2_88 | 852.5768 |
| siRNA_b2_89 | 704.5336 |
| siRNA_b2_90 | 2035.4359 |
| siRNA_b2_91 | 102.3794 |
| siRNA_b2_92 | 339.5151 |
| siRNA_b2_93 | 1021.5631 |
| siRNA_b2_94 | 1728.1956 |
| siRNA_b2_95 | 1245.0449 |
| siRNA_b2_96 | 1776.5912 |
| siRNA_b2_97 | 1879.9393 |
| siRNA_b2_98 | 1266.3998 |
| siRNA_b2_99 | 1686.5519 |
| siRNA_b2_100 | 369.6992 |
| siRNA_b2_101 | 1066.0329 |
| siRNA_b2_102 | 793.4938 |
| siRNA_b2_103 | 594.3273 |
| siRNA_b2_104 | 301.6089 |
| siRNA_b2_105 | 1329.1188 |
| siRNA_b2_106 | 777.4824 |
| siRNA_b2_107 | 937.9952 |
| siRNA_b2_108 | 113.5990 |
| siRNA_b2_109 | 1894.6330 |
| siRNA_b2_110 | 359.1919 |
| siRNA_b2_111 | 283.6114 |
| siRNA_b2_112 | 228.9139 |
| siRNA_b2_113 | 310.3502 |
| siRNA_b2_114 | 257.0341 |
| siRNA_b2_115 | 947.8835 |
| siRNA_b2_116 | 450.6404 |
| siRNA_b2_117 | 220.8653 |
| siRNA_b2_118 | 871.6906 |
| siRNA_b2_119 | 1016.9447 |
| siRNA_b2_120 | 238.6049 |
| siRNA_b2_121 | 1771.9314 |
| siRNA_b2_122 | 1218.0035 |
| siRNA_b2_123 | 614.7613 |
| siRNA_b2_124 | 801.3260 |
| siRNA_b2_125 | 199.9094 |
| siRNA_b2_126 | 226.2623 |
| siRNA_b2_127 | 173.8639 |
| siRNA_b2_128 | 335.3858 |
| siRNA_b2_129 | 184.2492 |
| siRNA_b2_130 | 613.3839 |
| siRNA_b2_131 | 145.1472 |
| siRNA_b2_132 | 854.7058 |
| siRNA_b2_133 | 217.1070 |
| siRNA_b2_134 | 143.1031 |
| siRNA_b2_135 | 873.5196 |
| siRNA_b2_136 | 94.2909 |
| siRNA_b2_137 | 251.1246 |
| siRNA_b2_138 | 976.1043 |
| siRNA_b2_139 | 684.5368 |
| siRNA_b2_140 | 359.8554 |
| siRNA_b2_141 | 1239.9926 |
| siRNA_b2_142 | 375.0238 |
| siRNA_b2_143 | 79.1307 |
| siRNA_b2_144 | 509.0688 |
| siRNA_b2_145 | 440.2141 |
| siRNA_b2_146 | 216.4884 |
| siRNA_b2_147 | 623.5351 |
| siRNA_b2_148 | 524.3706 |
| siRNA_b2_149 | 861.0373 |
| siRNA_b2_150 | 1250.1026 |
| siRNA_b2_151 | 249.7756 |
| siRNA_b2_152 | 1383.8970 |
| siRNA_b2_153 | 565.2681 |
| siRNA_b2_154 | 681.0833 |
| siRNA_b2_155 | 218.6192 |
| siRNA_b2_156 | 1177.5091 |
| siRNA_b2_157 | 647.2324 |
| siRNA_b2_158 | 562.3809 |
| siRNA_b2_159 | 97.5779 |
| siRNA_b2_160 | 264.6341 |
| siRNA_b2_161 | 978.4678 |
| siRNA_b2_162 | 602.8606 |
| siRNA_b2_163 | 992.2884 |
| siRNA_b2_164 | 525.3733 |
| siRNA_b2_165 | 1216.0120 |
| siRNA_b2_166 | 192.7664 |
| siRNA_b2_167 | 888.8690 |
| siRNA_b2_168 | 839.1989 |
| siRNA_b2_169 | 117.0361 |
| siRNA_b2_170 | 418.6384 |
| siRNA_b2_171 | 434.0471 |
| siRNA_b2_172 | 195.7624 |
| siRNA_b2_173 | 1959.7217 |
| siRNA_b2_174 | 841.2358 |
| siRNA_b2_175 | 544.5883 |
| siRNA_b2_176 | 322.7122 |
| siRNA_b2_177 | 57.1711 |
| siRNA_b2_178 | 516.6958 |
| siRNA_b2_179 | 873.1867 |
| siRNA_b2_180 | 439.2984 |

(3) Calculating Core Genomic Eigenvalues Based on Core Gene Weights and Off-Target Weights of all the Selected Off-Target Genes

Currently, it is known that more than 1,500 core genes have been discovered. For example, if four off-target genes A′, B′, C′, and D′ were identified, among which B′ and C′ were determined as core genes based on the known core genes, then their core genomic eigenvalue was counted as the sum of off-target weights of B′ and C′. The calculation results of the core genomic eigenvalues of the off-target genes of the respective siRNAs are shown in Table 14.

TABLE 14
Calculation results of core genomic eigenvalues
of off-target genes of siRNA

| siRNA name | Core genomic eigenvalue |
|---|---|
| siRNA_b2_1 | 8.1707 |
| siRNA_b2_2 | 9.5388 |
| siRNA_b2_3 | 5.1916 |
| siRNA_b2_4 | 5.9900 |
| siRNA_b2_5 | 11.4748 |
| siRNA_b2_6 | 22.5891 |
| siRNA_b2_7 | 16.1933 |
| siRNA_b2_8 | 7.2038 |
| siRNA_b2_9 | 3.9168 |
| siRNA_b2_10 | 11.9112 |
| siRNA_b2_11 | 9.5063 |
| siRNA_b2_12 | 1.2146 |
| siRNA_b2_13 | 16.0153 |
| siRNA_b2_14 | 11.0943 |
| siRNA_b2_15 | 9.6688 |
| siRNA_b2_16 | 7.5973 |
| siRNA_b2_17 | 7.4914 |
| siRNA_b2_18 | 9.8785 |
| siRNA_b2_19 | 6.1612 |
| siRNA_b2_20 | 5.2268 |
| siRNA_b2_21 | 4.5989 |
| siRNA_b2_22 | 9.1788 |
| siRNA_b2_23 | 7.7540 |
| siRNA_b2_24 | 3.7810 |
| siRNA_b2_25 | 8.7856 |
| siRNA_b2_26 | 3.5358 |
| siRNA_b2_27 | 1.8723 |
| siRNA_b2_28 | 10.8018 |
| siRNA_b2_29 | 15.0399 |
| siRNA_b2_30 | 6.8268 |
| siRNA_b2_31 | 15.2276 |
| siRNA_b2_32 | 8.3677 |
| siRNA_b2_33 | 10.2304 |
| siRNA_b2_34 | 9.9057 |
| siRNA_b2_35 | 7.4634 |
| siRNA_b2_36 | 13.1125 |
| siRNA_b2_37 | 13.1258 |
| siRNA_b2_38 | 7.8180 |
| siRNA_b2_39 | 11.6604 |
| siRNA_b2_40 | 7.4520 |
| siRNA_b2_41 | 5.5833 |
| siRNA_b2_42 | 7.0382 |
| siRNA_b2_43 | 5.5745 |
| siRNA_b2_44 | 12.6668 |
| siRNA_b2_45 | 2.7333 |
| siRNA_b2_46 | 11.2450 |
| siRNA_b2_47 | 15.3387 |
| siRNA_b2_48 | 13.0992 |
| siRNA_b2_49 | 3.2389 |
| siRNA_b2_50 | 13.4239 |
| siRNA_b2_51 | 9.5082 |
| siRNA_b2_52 | 3.4489 |
| siRNA_b2_53 | 3.2098 |
| siRNA_b2_54 | 3.8131 |
| siRNA_b2_55 | 6.4982 |
| siRNA_b2_56 | 4.0239 |
| siRNA_b2_57 | 3.4432 |
| siRNA_b2_58 | 3.7564 |
| siRNA_b2_59 | 9.9064 |
| siRNA_b2_60 | 3.2706 |
| siRNA_b2_61 | 5.6872 |
| siRNA_b2_62 | 9.0017 |
| siRNA_b2_63 | 9.2355 |
| siRNA_b2_64 | 2.2536 |
| siRNA_b2_65 | 10.3460 |
| siRNA_b2_66 | 7.3481 |
| siRNA_b2_67 | 8.0696 |
| siRNA_b2_68 | 8.0717 |
| siRNA_b2_69 | 14.2589 |
| siRNA_b2_70 | 10.5845 |
| siRNA_b2_71 | 6.6039 |
| siRNA_b2_72 | 2.0957 |
| siRNA_b2_73 | 7.4512 |
| siRNA_b2_74 | 3.8613 |
| siRNA_b2_75 | 7.7669 |
| siRNA_b2_76 | 2.7680 |
| siRNA_b2_77 | 6.4713 |
| siRNA_b2_78 | 6.9631 |
| siRNA_b2_79 | 10.2533 |
| siRNA_b2_80 | 6.7240 |
| siRNA_b2_81 | 11.5937 |
| siRNA_b2_82 | 7.2580 |
| siRNA_b2_83 | 9.5668 |
| siRNA_b2_84 | 14.3023 |
| siRNA_b2_85 | 4.0694 |
| siRNA_b2_86 | 3.5063 |
| siRNA_b2_87 | 12.4124 |
| siRNA_b2_88 | 8.0267 |
| siRNA_b2_89 | 8.6750 |
| siRNA_b2_90 | 24.8349 |
| siRNA_b2_91 | 1.7435 |
| siRNA_b2_92 | 5.3378 |
| siRNA_b2_93 | 9.4694 |
| siRNA_b2_94 | 16.8028 |
| siRNA_b2_95 | 8.4053 |
| siRNA_b2_96 | 13.7846 |
| siRNA_b2_97 | 15.5149 |
| siRNA_b2_98 | 13.7002 |
| siRNA_b2_99 | 14.4165 |
| siRNA_b2_100 | 2.7452 |
| siRNA_b2_101 | 11.7968 |
| siRNA_b2_102 | 10.3150 |
| siRNA_b2_103 | 7.7677 |
| siRNA_b2_104 | 5.9699 |
| siRNA_b2_105 | 12.0875 |
| siRNA_b2_106 | 8.0230 |
| siRNA_b2_107 | 11.5738 |
| siRNA_b2_108 | 2.7836 |
| siRNA_b2_109 | 15.7147 |
| siRNA_b2_110 | 1.1218 |
| siRNA_b2_111 | 5.7817 |
| siRNA_b2_112 | 2.9587 |
| siRNA_b2_113 | 2.9516 |
| siRNA_b2_114 | 1.3518 |
| siRNA_b2_115 | 7.8368 |
| siRNA_b2_116 | 7.8214 |
| siRNA_b2_117 | 2.7959 |
| siRNA_b2_118 | 4.0744 |
| siRNA_b2_119 | 13.9697 |
| siRNA_b2_120 | 4.1764 |
| siRNA_b2_121 | 13.9923 |
| siRNA_b2_122 | 10.7201 |
| siRNA_b2_123 | 4.4526 |
| siRNA_b2_124 | 7.7829 |
| siRNA_b2_125 | 3.4457 |
| siRNA_b2_126 | 4.1994 |
| siRNA_b2_127 | 2.5988 |
| siRNA_b2_128 | 2.7438 |
| siRNA_b2_129 | 1.4850 |
| siRNA_b2_130 | 9.2745 |
| siRNA_b2_131 | 2.9583 |
| siRNA_b2_132 | 10.8458 |
| siRNA_b2_133 | 4.2707 |
| siRNA_b2_134 | 2.0529 |
| siRNA_b2_135 | 8.7911 |
| siRNA_b2_136 | 1.0037 |
| siRNA_b2_137 | 2.0015 |
| siRNA_b2_138 | 2.6510 |
| siRNA_b2_139 | 10.9706 |
| siRNA_b2_140 | 4.7423 |
| siRNA_b2_141 | 6.1324 |
| siRNA_b2_142 | 3.7264 |
| siRNA_b2_143 | 0.7353 |
| siRNA_b2_144 | 5.6007 |
| siRNA_b2_145 | 3.7980 |
| siRNA_b2_146 | 2.5509 |
| siRNA_b2_147 | 2.7660 |
| siRNA_b2_148 | 2.6819 |
| siRNA_b2_149 | 8.3747 |
| siRNA_b2_150 | 13.7168 |
| siRNA_b2_151 | 3.4709 |
| siRNA_b2_152 | 12.0852 |
| siRNA_b2_153 | 7.8085 |
| siRNA_b2_154 | 9.8225 |
| siRNA_b2_155 | 2.5669 |
| siRNA_b2_156 | 10.0750 |
| siRNA_b2_157 | 5.8920 |
| siRNA_b2_158 | 3.7694 |
| siRNA_b2_159 | 2.5910 |
| siRNA_b2_160 | 4.5370 |
| siRNA_b2_161 | 10.3915 |
| siRNA_b2_162 | 5.7073 |
| siRNA_b2_163 | 9.1004 |
| siRNA_b2_164 | 8.2752 |
| siRNA_b2_165 | 11.0770 |
| siRNA_b2_166 | 4.6614 |
| siRNA_b2_167 | 8.6529 |
| siRNA_b2_168 | 9.5371 |
| siRNA_b2_169 | 0.6199 |
| siRNA_b2_170 | 6.8498 |
| siRNA_b2_171 | 4.1564 |
| siRNA_b2_172 | 4.4022 |
| siRNA_b2_173 | 13.4973 |
| siRNA_b2_174 | 6.2235 |
| siRNA_b2_175 | 5.2230 |
| siRNA_b2_176 | 3.0514 |
| siRNA_b2_177 | 0.4708 |
| siRNA_b2_178 | 6.0173 |
| siRNA_b2_179 | 10.1567 |
| siRNA_b2_180 | 4.6271 |

The output value of any one of the 180 siRNAs was obtained as follows:

A549 cells were transfected with the above 180 siRNAs. There were also a blank group (not transfected), and a negative control group (transfected with a random siRNA sequence (synthesized by Invitrogen), i.e an siRNA not targeting at MGMT gene). After cultured for 48 hours under the culture conditions as described above in Experimental Example 1, the cells were treated with CCK-8 solution by adding 10 μL of CCK-8 solution to each well, and the plate was incubated in an incubator for 0.5 to 1 hour. The absorbance at 450 nm was measured by a microplate reader, and the OD450 data was collected. The ratio of the OD450 value of each experimental group to the OD450 value of the blank group was calculated to obtain the cell survival index of each siRNA. The results are shown in Table 15.

TABLE 15
Cell survival index of siRNA

| siRNA name | Cell survival index |
|---|---|
| siRNA_b2_1 | 0.9651 |
| siRNA_b2_2 | 0.5121 |
| siRNA_b2_3 | 0.6545 |
| siRNA_b2_4 | 0.6960 |
| siRNA_b2_5 | 0.9323 |
| siRNA_b2_6 | 0.6971 |
| siRNA_b2_7 | 0.8690 |
| siRNA_b2_8 | 0.9401 |
| siRNA_b2_9 | 0.7266 |
| siRNA_b2_10 | 0.3181 |
| siRNA_b2_11 | 0.8974 |
| siRNA_b2_12 | 1.0489 |
| siRNA_b2_13 | 0.8483 |
| siRNA_b2_14 | 0.9171 |
| siRNA_b2_15 | 0.6493 |
| siRNA_b2_16 | 0.5979 |
| siRNA_b2_17 | 0.8658 |
| siRNA_b2_18 | 0.8921 |
| siRNA_b2_19 | 0.5199 |
| siRNA_b2_20 | 0.7665 |
| siRNA_b2_21 | 0.8083 |
| siRNA_b2_22 | 0.6618 |
| siRNA_b2_23 | 0.9264 |
| siRNA_b2_24 | 0.9427 |
| siRNA_b2_25 | 0.9014 |
| siRNA_b2_26 | 0.8753 |
| siRNA_b2_27 | 0.6930 |
| siRNA_b2_28 | 1.0059 |
| siRNA_b2_29 | 0.6766 |
| siRNA_b2_30 | 0.6723 |
| siRNA_b2_31 | 0.5876 |
| siRNA_b2_32 | 0.8167 |
| siRNA_b2_33 | 0.9485 |
| siRNA_b2_34 | 0.7121 |
| siRNA_b2_35 | 0.8459 |
| siRNA_b2_36 | 0.7075 |
| siRNA_b2_37 | 0.3777 |
| siRNA_b2_38 | 0.8259 |
| siRNA_b2_39 | 0.7885 |
| siRNA_b2_40 | 1.1294 |
| siRNA_b2_41 | 0.5305 |
| siRNA_b2_42 | 0.5773 |
| siRNA_b2_43 | 0.5900 |
| siRNA_b2_44 | 0.7570 |
| siRNA_b2_45 | 0.6268 |
| siRNA_b2_46 | 0.5179 |
| siRNA_b2_47 | 0.6508 |
| siRNA_b2_48 | 0.9132 |
| siRNA_b2_49 | 0.7076 |
| siRNA_b2_50 | 0.7623 |
| siRNA_b2_51 | 0.1220 |
| siRNA_b2_52 | 0.1323 |
| siRNA_b2_53 | 0.1372 |
| siRNA_b2_54 | 0.6113 |
| siRNA_b2_55 | 0.1144 |
| siRNA_b2_56 | 0.7744 |
| siRNA_b2_57 | 0.6590 |
| siRNA_b2_58 | 0.6338 |
| siRNA_b2_59 | 0.6339 |
| siRNA_b2_60 | 0.9612 |
| siRNA_b2_61 | 1.0054 |
| siRNA_b2_62 | 0.7382 |
| siRNA_b2_63 | 1.1462 |
| siRNA_b2_64 | 1.1465 |
| siRNA_b2_65 | 0.9148 |
| siRNA_b2_66 | 0.6560 |
| siRNA_b2_67 | 0.9966 |
| siRNA_b2_68 | 0.5344 |
| siRNA_b2_69 | 0.6196 |
| siRNA_b2_70 | 0.7628 |
| siRNA_b2_71 | 0.7538 |
| siRNA_b2_72 | 0.9272 |
| siRNA_b2_73 | 0.7951 |
| siRNA_b2_74 | 0.3550 |
| siRNA_b2_75 | 0.6819 |
| siRNA_b2_76 | 0.9848 |
| siRNA_b2_77 | 1.1466 |
| siRNA_b2_78 | 0.6932 |
| siRNA_b2_79 | 0.9368 |
| siRNA_b2_80 | 0.9515 |
| siRNA_b2_81 | 0.7092 |
| siRNA_b2_82 | 0.4686 |
| siRNA_b2_83 | 0.6074 |
| siRNA_b2_84 | 0.8450 |
| siRNA_b2_85 | 0.3932 |
| siRNA_b2_86 | 0.4748 |
| siRNA_b2_87 | 0.7675 |
| siRNA_b2_88 | 0.5628 |
| siRNA_b2_89 | 0.5706 |
| siRNA_b2_90 | 0.6352 |
| siRNA_b2_91 | 0.5105 |
| siRNA_b2_92 | 0.7243 |
| siRNA_b2_93 | 0.6796 |
| siRNA_b2_94 | 0.8912 |
| siRNA_b2_95 | 0.7852 |
| siRNA_b2_96 | 1.0930 |
| siRNA_b2_97 | 0.8300 |
| siRNA_b2_98 | 0.8045 |
| siRNA_b2_99 | 0.7286 |
| siRNA_b2_100 | 0.7888 |
| siRNA_b2_101 | 1.0038 |
| siRNA_b2_102 | 0.9658 |
| siRNA_b2_103 | 0.7990 |
| siRNA_b2_104 | 0.9091 |
| siRNA_b2_105 | 0.6238 |
| siRNA_b2_106 | 0.7229 |
| siRNA_b2_107 | 1.0478 |
| siRNA_b2_108 | 0.8193 |
| siRNA_b2_109 | 1.0269 |
| siRNA_b2_110 | 0.8075 |
| siRNA_b2_111 | 0.8996 |
| siRNA_b2_112 | 1.0291 |
| siRNA_b2_113 | 0.6584 |
| siRNA_b2_114 | 0.8459 |
| siRNA_b2_115 | 0.9868 |
| siRNA_b2_116 | 0.7741 |
| siRNA_b2_117 | 0.6537 |
| siRNA_b2_118 | 0.7232 |
| siRNA_b2_119 | 0.8188 |
| siRNA_b2_120 | 0.8483 |
| siRNA_b2_121 | 0.6629 |
| siRNA_b2_122 | 0.5854 |
| siRNA_b2_123 | 0.6220 |
| siRNA_b2_124 | 0.7292 |
| siRNA_b2_125 | 0.3776 |
| siRNA_b2_126 | 0.5967 |
| siRNA_b2_127 | 0.8062 |
| siRNA_b2_128 | 0.6491 |
| siRNA_b2_129 | 0.8902 |
| siRNA_b2_130 | 0.8176 |
| siRNA_b2_131 | 0.9055 |
| siRNA_b2_132 | 0.7840 |
| siRNA_b2_133 | 0.8136 |
| siRNA_b2_134 | 0.9283 |
| siRNA_b2_135 | 0.8564 |
| siRNA_b2_136 | 1.0407 |
| siRNA_b2_137 | 0.9099 |
| siRNA_b2_138 | 0.9448 |
| siRNA_b2_139 | 0.8918 |
| siRNA_b2_140 | 0.7869 |
| siRNA_b2_141 | 0.9723 |
| siRNA_b2_142 | 1.0374 |
| siRNA_b2_143 | 0.9474 |
| siRNA_b2_144 | 0.9039 |
| siRNA_b2_145 | 1.0779 |
| siRNA_b2_146 | 1.0119 |
| siRNA_b2_147 | 0.8232 |
| siRNA_b2_148 | 0.9235 |
| siRNA_b2_149 | 0.5952 |
| siRNA_b2_150 | 0.6710 |
| siRNA_b2_151 | 0.8793 |
| siRNA_b2_152 | 1.0398 |
| siRNA_b2_153 | 0.6989 |
| siRNA_b2_154 | 0.7716 |
| siRNA_b2_155 | 0.8018 |
| siRNA_b2_156 | 1.0453 |
| siRNA_b2_157 | 1.0031 |
| siRNA_b2_158 | 0.9172 |
| siRNA_b2_159 | 0.7935 |
| siRNA_b2_160 | 0.4756 |
| siRNA_b2_161 | 0.8171 |
| siRNA_b2_162 | 0.7625 |
| siRNA_b2_163 | 0.2820 |
| siRNA_b2_164 | 0.6544 |
| siRNA_b2_165 | 0.4089 |
| siRNA_b2_166 | 0.6017 |
| siRNA_b2_167 | 0.9457 |
| siRNA_b2_168 | 0.8091 |
| siRNA_b2_169 | 0.7952 |
| siRNA_b2_170 | 0.4399 |
| siRNA_b2_171 | 0.6030 |
| siRNA_b2_172 | 1.0037 |
| siRNA_b2_173 | 0.4540 |
| siRNA_b2_174 | 0.7301 |
| siRNA_b2_175 | 0.4972 |
| siRNA_b2_176 | 0.4539 |
| siRNA_b2_177 | 0.6081 |
| siRNA_b2_178 | 0.4329 |
| siRNA_b2_179 | 0.7494 |
| siRNA_b2_180 | 0.7833 |

C. Establishing a Machine Learning Model Through Machine Learning Algorithm

In this embodiment, the KNIME® Analytics Platform software was selected to construct a machine learning model. The KNIME® Analytics Platform is an integrated software for open operation developed by KNIME of Switzerland for data-driven innovation. A description of the KNIME® Analytics Platform software can be found in the literature: “Berthold, M. R., Cebron, N., Dill, F., Gabriel, T. R., Kotter, T., Meinl, T., Ohl, P., Sieb, C., Thiel, K., Wiswedel, B.: KNIME: The Konstanz Information Miner. In: Studies in Classification, Data Analysis, and Knowledge Organization (GfKL 2007). Springer (2007)”, the entire content of which is incorporated herein by reference. The KNIME® Analytics Platform has more than a thousand modules, hundreds of ready-to-run examples, comprehensive integration tools, and the broadest selection of advanced algorithms, integrating open source projects such as machine learning algorithms, R and chemical development kits. It is the preferred toolbox for most data scientists. Therefore, in this example the KNIME® Analytics Platform software was selected to establish a machine learning model.

(1) Establishing a Machine Learning Model Through the Machine Learning Algorithm PNN

As described above, the proteomic eigenvalues, signal pathwayomic eigenvalues, and core genomic eigenvalues were obtained for a specific siRNA. These data need to be normalized before being used as input values for machine learning algorithms. The data were mapped one-to-one to the interval 0-1 using the formula: (a value-minimum)/(maximum-minimum). The normalization of the above data can be achieved by the Normalizer node in the KNIME® Analytics Platform software. The results of the normalized proteomic eigenvalues, signal pathwayomic eigenvalues, and core genomic eigenvalues are shown in Table 16.

TABLE 16
Proteomic eigenvalues, signal pathwayomic eigenvalues, and
core genomic eigenvalues results after normalization

| | Normalized | Normalized | Normalized |
|---|---|---|---|
| | proteomic | signal pathwayomic | core genomic |
| siRNA name | eigenvalues | eigenvalues | eigenvalues |
| siRNA_b2_1 | 0.0528 | 0.1316 | 0.3160 |
| siRNA_b2_2 | 0.0970 | 0.1892 | 0.3722 |
| siRNA_b2_3 | 0.0151 | 0.0707 | 0.1938 |
| siRNA_b2_4 | 0.0834 | 0.2056 | 0.2265 |
| siRNA_b2_5 | 0.1234 | 0.1660 | 0.4516 |
| siRNA_b2_6 | 1.0000 | 1.0000 | 0.9078 |
| siRNA_b2_7 | 0.3206 | 0.3637 | 0.6453 |
| siRNA_b2_8 | 0.0540 | 0.1189 | 0.2763 |
| siRNA_b2_9 | 0.0292 | 0.1203 | 0.1414 |
| siRNA_b2_10 | 0.1740 | 0.2895 | 0.4696 |
| siRNA_b2_11 | 0.1147 | 0.2042 | 0.3709 |
| siRNA_b2_12 | 0.0060 | 0.0517 | 0.0305 |
| siRNA_b2_13 | 0.2719 | 0.4433 | 0.6380 |
| siRNA_b2_14 | 0.0763 | 0.1700 | 0.4360 |
| siRNA_b2_15 | 0.1020 | 0.1878 | 0.3775 |
| siRNA_b2_16 | 0.0580 | 0.1808 | 0.2925 |
| siRNA_b2_17 | 0.1017 | 0.1907 | 0.2882 |
| siRNA_b2_18 | 0.1512 | 0.3701 | 0.3861 |
| siRNA_b2_19 | 0.0771 | 0.1984 | 0.2336 |
| siRNA_b2_20 | 0.0567 | 0.1531 | 0.1952 |
| siRNA_b2_21 | 0.0386 | 0.1671 | 0.1694 |
| siRNA_b2_22 | 0.1723 | 0.3570 | 0.3574 |
| siRNA_b2_23 | 0.0452 | 0.1431 | 0.2989 |
| siRNA_b2_24 | 0.0260 | 0.0762 | 0.1359 |
| siRNA_b2_25 | 0.0582 | 0.1309 | 0.3413 |
| siRNA_b2_26 | 0.0216 | 0.0937 | 0.1258 |
| siRNA_b2_27 | 0.0019 | 0.0525 | 0.0575 |
| siRNA_b2_28 | 0.1523 | 0.2423 | 0.4240 |
| siRNA_b2_29 | 0.2312 | 0.2700 | 0.5980 |
| siRNA_b2_30 | 0.0350 | 0.1245 | 0.2609 |
| siRNA_b2_31 | 0.2932 | 0.3017 | 0.6057 |
| siRNA_b2_32 | 0.0798 | 0.1804 | 0.3241 |
| siRNA_b2_33 | 0.1700 | 0.2492 | 0.4006 |
| siRNA_b2_34 | 0.1665 | 0.2888 | 0.3872 |
| siRNA_b2_35 | 0.0730 | 0.2450 | 0.2870 |
| siRNA_b2_36 | 0.1849 | 0.3671 | 0.5189 |
| siRNA_b2_37 | 0.2109 | 0.4334 | 0.5194 |
| siRNA_b2_38 | 0.0960 | 0.3720 | 0.3016 |
| siRNA_b2_39 | 0.2021 | 0.3516 | 0.4593 |
| siRNA_b2_40 | 0.0590 | 0.1572 | 0.2865 |
| siRNA_b2_41 | 0.0364 | 0.2033 | 0.2098 |
| siRNA_b2_42 | 0.0378 | 0.1439 | 0.2696 |
| siRNA_b2_43 | 0.0214 | 0.1139 | 0.2095 |
| siRNA_b2_44 | 0.2490 | 0.3646 | 0.5006 |
| siRNA_b2_45 | 0.0062 | 0.0713 | 0.0929 |
| siRNA_b2_46 | 0.1350 | 0.1784 | 0.4422 |
| siRNA_b2_47 | 0.2707 | 0.4774 | 0.6102 |
| siRNA_b2_48 | 0.1097 | 0.1891 | 0.5183 |
| siRNA_b2_49 | 0.0110 | 0.0175 | 0.1136 |
| siRNA_b2_50 | 0.1377 | 0.3173 | 0.5316 |
| siRNA_b2_51 | 0.1113 | 0.2272 | 0.3709 |
| siRNA_b2_52 | 0.0084 | 0.0599 | 0.1222 |
| siRNA_b2_53 | 0.0154 | 0.1078 | 0.1124 |
| siRNA_b2_54 | 0.0079 | 0.0597 | 0.1372 |
| siRNA_b2_55 | 0.0334 | 0.0794 | 0.2474 |
| siRNA_b2_56 | 0.0238 | 0.1803 | 0.1458 |
| siRNA_b2_57 | 0.0232 | 0.0775 | 0.1220 |
| siRNA_b2_58 | 0.0074 | 0.0579 | 0.1349 |
| siRNA_b2_59 | 0.1313 | 0.2226 | 0.3873 |
| siRNA_b2_60 | 0.0111 | 0.1131 | 0.1149 |
| siRNA_b2_61 | 0.0338 | 0.0970 | 0.2141 |
| siRNA_b2_62 | 0.1609 | 0.2837 | 0.3501 |
| siRNA_b2_63 | 0.0485 | 0.2202 | 0.3597 |
| siRNA_b2_64 | 0.0123 | 0.0714 | 0.0732 |
| siRNA_b2_65 | 0.1132 | 0.2303 | 0.4053 |
| siRNA_b2_66 | 0.0923 | 0.1955 | 0.2823 |
| siRNA_b2_67 | 0.0310 | 0.1000 | 0.3119 |
| siRNA_b2_68 | 0.1033 | 0.2205 | 0.3120 |
| siRNA_b2_69 | 0.1475 | 0.3288 | 0.5659 |
| siRNA_b2_70 | 0.1327 | 0.2276 | 0.4151 |
| siRNA_b2_71 | 0.0328 | 0.1179 | 0.2517 |
| siRNA_b2_72 | 0.0181 | 0.0728 | 0.0667 |
| siRNA_b2_73 | 0.1045 | 0.3265 | 0.2865 |
| siRNA_b2_74 | 0.0349 | 0.1979 | 0.1392 |
| siRNA_b2_75 | 0.0998 | 0.4182 | 0.2995 |
| siRNA_b2_76 | 0.0324 | 0.1471 | 0.0943 |
| siRNA_b2_77 | 0.0329 | 0.1298 | 0.2463 |
| siRNA_b2_78 | 0.0547 | 0.2042 | 0.2665 |
| siRNA_b2_79 | 0.2009 | 0.3190 | 0.4015 |
| siRNA_b2_80 | 0.0584 | 0.1511 | 0.2567 |
| siRNA_b2_81 | 0.1368 | 0.2536 | 0.4565 |
| siRNA_b2_82 | 0.0335 | 0.1424 | 0.2786 |
| siRNA_b2_83 | 0.0664 | 0.1581 | 0.3733 |
| siRNA_b2_84 | 0.2649 | 0.3733 | 0.5677 |
| siRNA_b2_85 | 0.0464 | 0.1435 | 0.1477 |
| siRNA_b2_86 | 0.0058 | 0.0745 | 0.1246 |
| siRNA_b2_87 | 0.2494 | 0.3381 | 0.4901 |
| siRNA_b2_88 | 0.0438 | 0.2100 | 0.3101 |
| siRNA_b2_89 | 0.0632 | 0.1709 | 0.3367 |
| siRNA_b2_90 | 0.5255 | 0.5223 | 1.0000 |
| siRNA_b2_91 | 0.0013 | 0.0119 | 0.0522 |
| siRNA_b2_92 | 0.0244 | 0.0745 | 0.1998 |
| siRNA_b2_93 | 0.1112 | 0.2546 | 0.3693 |
| siRNA_b2_94 | 0.4798 | 0.4412 | 0.6703 |
| siRNA_b2_95 | 0.0884 | 0.3136 | 0.3257 |
| siRNA_b2_96 | 0.3138 | 0.4540 | 0.5465 |
| siRNA_b2_97 | 0.4036 | 0.4812 | 0.6175 |
| siRNA_b2_98 | 0.2542 | 0.3193 | 0.5430 |
| siRNA_b2_99 | 0.3149 | 0.4302 | 0.5724 |
| siRNA_b2_100 | 0.0111 | 0.0825 | 0.0933 |
| siRNA_b2_101 | 0.1547 | 0.2664 | 0.4649 |
| siRNA_b2_102 | 0.1226 | 0.1944 | 0.4040 |
| siRNA_b2_103 | 0.0618 | 0.1418 | 0.2995 |
| siRNA_b2_104 | 0.0217 | 0.0645 | 0.2257 |
| siRNA_b2_105 | 0.2117 | 0.3358 | 0.4768 |
| siRNA_b2_106 | 0.0856 | 0.1902 | 0.3100 |
| siRNA_b2_107 | 0.1446 | 0.2326 | 0.4557 |
| siRNA_b2_108 | 0.0057 | 0.0149 | 0.0949 |
| siRNA_b2_109 | 0.3604 | 0.4851 | 0.6257 |
| siRNA_b2_110 | 0.0088 | 0.0797 | 0.0267 |
| siRNA_b2_111 | 0.0400 | 0.0598 | 0.2180 |
| siRNA_b2_112 | 0.0073 | 0.0453 | 0.1021 |
| siRNA_b2_113 | 0.0110 | 0.0668 | 0.1018 |
| siRNA_b2_114 | 0.0065 | 0.0528 | 0.0362 |
| siRNA_b2_115 | 0.1305 | 0.2352 | 0.3023 |
| siRNA_b2_116 | 0.0907 | 0.1039 | 0.3017 |
| siRNA_b2_117 | 0.0157 | 0.0432 | 0.0954 |
| siRNA_b2_118 | 0.0168 | 0.2151 | 0.1479 |
| siRNA_b2_119 | 0.0901 | 0.2534 | 0.5540 |
| siRNA_b2_120 | 0.0073 | 0.0479 | 0.1521 |
| siRNA_b2_121 | 0.1766 | 0.4527 | 0.5550 |
| siRNA_b2_122 | 0.1597 | 0.3065 | 0.4207 |
| siRNA_b2_123 | 0.0468 | 0.1472 | 0.1634 |
| siRNA_b2_124 | 0.0675 | 0.1965 | 0.3001 |
| siRNA_b2_125 | 0.0112 | 0.0377 | 0.1221 |
| siRNA_b2_126 | 0.0142 | 0.0446 | 0.1530 |
| siRNA_b2_127 | 0.0035 | 0.0308 | 0.0873 |
| siRNA_b2_128 | 0.0161 | 0.0735 | 0.0933 |
| siRNA_b2_129 | 0.0041 | 0.0336 | 0.0416 |
| siRNA_b2_130 | 0.0870 | 0.1469 | 0.3613 |
| siRNA_b2_131 | 0.0089 | 0.0232 | 0.1021 |
| siRNA_b2_132 | 0.0939 | 0.2106 | 0.4258 |
| siRNA_b2_133 | 0.0091 | 0.0422 | 0.1560 |
| siRNA_b2_134 | 0.0035 | 0.0227 | 0.0649 |
| siRNA_b2_135 | 0.0766 | 0.2155 | 0.3415 |
| siRNA_b2_136 | 0.0019 | 0.0098 | 0.0219 |
| siRNA_b2_137 | 0.0084 | 0.0512 | 0.0628 |
| siRNA_b2_138 | 0.0204 | 0.2426 | 0.0895 |
| siRNA_b2_139 | 0.1165 | 0.1656 | 0.4310 |
| siRNA_b2_140 | 0.0234 | 0.0799 | 0.1753 |
| siRNA_b2_141 | 0.0861 | 0.3123 | 0.2324 |
| siRNA_b2_142 | 0.0370 | 0.0839 | 0.1336 |
| siRNA_b2_143 | 0.0000 | 0.0058 | 0.0109 |
| siRNA_b2_144 | 0.0268 | 0.1193 | 0.2106 |
| siRNA_b2_145 | 0.0195 | 0.1011 | 0.1366 |
| siRNA_b2_146 | 0.0053 | 0.0421 | 0.0854 |
| siRNA_b2_147 | 0.0075 | 0.1495 | 0.0942 |
| siRNA_b2_148 | 0.0075 | 0.1234 | 0.0908 |
| siRNA_b2_149 | 0.0504 | 0.2122 | 0.3244 |
| siRNA_b2_150 | 0.2224 | 0.3150 | 0.5437 |
| siRNA_b2_151 | 0.0065 | 0.0509 | 0.1231 |
| siRNA_b2_152 | 0.1734 | 0.3503 | 0.4767 |
| siRNA_b2_153 | 0.0529 | 0.1341 | 0.3012 |
| siRNA_b2_154 | 0.0685 | 0.1647 | 0.3838 |
| siRNA_b2_155 | 0.0104 | 0.0426 | 0.0860 |
| siRNA_b2_156 | 0.1036 | 0.2958 | 0.3942 |
| siRNA_b2_157 | 0.0523 | 0.1558 | 0.2225 |
| siRNA_b2_158 | 0.0583 | 0.1334 | 0.1354 |
| siRNA_b2_159 | 0.0029 | 0.0107 | 0.0870 |
| siRNA_b2_160 | 0.0126 | 0.0548 | 0.1669 |
| siRNA_b2_161 | 0.1768 | 0.2432 | 0.4072 |
| siRNA_b2_162 | 0.0434 | 0.1441 | 0.2149 |
| siRNA_b2_163 | 0.1229 | 0.2469 | 0.3542 |
| siRNA_b2_164 | 0.0551 | 0.1236 | 0.3203 |
| siRNA_b2_165 | 0.1509 | 0.3060 | 0.4353 |
| siRNA_b2_166 | 0.0120 | 0.0358 | 0.1720 |
| siRNA_b2_167 | 0.1125 | 0.2196 | 0.3358 |
| siRNA_b2_168 | 0.0726 | 0.2065 | 0.3721 |
| siRNA_b2_169 | 0.0014 | 0.0158 | 0.0061 |
| siRNA_b2_170 | 0.0477 | 0.0954 | 0.2618 |
| siRNA_b2_171 | 0.0235 | 0.0995 | 0.1513 |
| siRNA_b2_172 | 0.0190 | 0.0366 | 0.1614 |
| siRNA_b2_173 | 0.3856 | 0.5023 | 0.5347 |
| siRNA_b2_174 | 0.0416 | 0.2070 | 0.2361 |
| siRNA_b2_175 | 0.0670 | 0.1287 | 0.1950 |
| siRNA_b2_176 | 0.0098 | 0.0701 | 0.1059 |
| siRNA_b2_177 | 0.0019 | 0.0000 | 0.0000 |
| siRNA_b2_178 | 0.0259 | 0.1213 | 0.2277 |
| siRNA_b2_179 | 0.1035 | 0.2154 | 0.3975 |
| siRNA_b2_180 | 0.0254 | 0.1009 | 0.1706 |

For the output value data of the machine learning algorithm, that is, the survival indexes of the cells in the presence of the siRNA, they were binarized before being used as the output value data (for example, with a survival index of 0.75 as a boundary value, those higher than or equal to 0.75 being set to y, and the rest being set to n). The cell survival index results after the binarization treatment are shown in Table 17.

TABLE 17
Cell survival index results after binarization

| siRNA name | Binarized cell survival index |
|---|---|
| siRNA_b2_1 | y |
| siRNA_b2_2 | n |
| siRNA_b2_3 | n |
| siRNA_b2_4 | n |
| siRNA_b2_5 | y |
| siRNA_b2_6 | n |
| siRNA_b2_7 | y |
| siRNA_b2_8 | y |
| siRNA_b2_9 | n |
| siRNA_b2_10 | n |
| siRNA_b2_11 | y |
| siRNA_b2_12 | y |
| siRNA_b2_13 | y |
| siRNA_b2_14 | y |
| siRNA_b2_15 | n |
| siRNA_b2_16 | n |
| siRNA_b2_17 | y |
| siRNA_b2_18 | y |
| siRNA_b2_19 | n |
| siRNA_b2_20 | y |
| siRNA_b2_21 | y |
| siRNA_b2_22 | n |
| siRNA_b2_23 | y |
| siRNA_b2_24 | y |
| siRNA_b2_25 | y |
| siRNA_b2_26 | y |
| siRNA_b2_27 | n |
| siRNA_b2_28 | y |
| siRNA_b2_29 | n |
| siRNA_b2_30 | n |
| siRNA_b2_31 | n |
| siRNA_b2_32 | y |
| siRNA_b2_33 | y |
| siRNA_b2_34 | n |
| siRNA_b2_35 | y |
| siRNA_b2_36 | n |
| siRNA_b2_37 | n |
| siRNA_b2_38 | y |
| siRNA_b2_39 | y |
| siRNA_b2_40 | y |
| siRNA_b2_41 | n |
| siRNA_b2_42 | n |
| siRNA_b2_43 | n |
| siRNA_b2_44 | y |
| siRNA_b2_45 | n |
| siRNA_b2_46 | n |
| siRNA_b2_47 | n |
| siRNA_b2_48 | y |
| siRNA_b2_49 | n |
| siRNA_b2_50 | y |
| siRNA_b2_51 | n |
| siRNA_b2_52 | n |
| siRNA_b2_53 | n |
| siRNA_b2_54 | n |
| siRNA_b2_55 | n |
| siRNA_b2_56 | y |
| siRNA_b2_57 | n |
| siRNA_b2_58 | n |
| siRNA_b2_59 | n |
| siRNA_b2_60 | y |
| siRNA_b2_61 | y |
| siRNA_b2_62 | n |
| siRNA_b2_63 | y |
| siRNA_b2_64 | y |
| siRNA_b2_65 | y |
| siRNA_b2_66 | n |
| siRNA_b2_67 | y |
| siRNA_b2_68 | n |
| siRNA_b2_69 | n |
| siRNA_b2_70 | y |
| siRNA_b2_71 | y |
| siRNA_b2_72 | y |
| siRNA_b2_73 | y |
| siRNA_b2_74 | n |
| siRNA_b2_75 | n |
| siRNA_b2_76 | y |
| siRNA_b2_77 | y |
| siRNA_b2_78 | n |
| siRNA_b2_79 | y |
| siRNA_b2_80 | y |
| siRNA_b2_81 | n |
| siRNA_b2_82 | n |
| siRNA_b2_83 | n |
| siRNA_b2_84 | y |
| siRNA_b2_85 | n |
| siRNA_b2_86 | n |
| siRNA_b2_87 | y |
| siRNA_b2_88 | n |
| siRNA_b2_89 | n |
| siRNA_b2_90 | n |
| siRNA_b2_91 | n |
| siRNA_b2_92 | n |
| siRNA_b2_93 | n |
| siRNA_b2_94 | y |
| siRNA_b2_95 | y |
| siRNA_b2_96 | y |
| siRNA_b2_97 | y |
| siRNA_b2_98 | y |
| siRNA_b2_99 | n |
| siRNA_b2_100 | y |
| siRNA_b2_101 | y |
| siRNA_b2_102 | y |
| siRNA_b2_103 | y |
| siRNA_b2_104 | y |
| siRNA_b2_105 | n |
| siRNA_b2_106 | n |
| siRNA_b2_107 | y |
| siRNA_b2_108 | y |
| siRNA_b2_109 | y |
| siRNA_b2_110 | y |
| siRNA_b2_111 | y |
| siRNA_b2_112 | y |
| siRNA_b2_113 | n |
| siRNA_b2_114 | y |
| siRNA_b2_115 | y |
| siRNA_b2_116 | y |
| siRNA_b2_117 | n |
| siRNA_b2_118 | n |
| siRNA_b2_119 | y |
| siRNA_b2_120 | y |
| siRNA_b2_121 | n |
| siRNA_b2_122 | n |
| siRNA_b2_123 | n |
| siRNA_b2_124 | n |
| siRNA_b2_125 | n |
| siRNA_b2_126 | n |
| siRNA_b2_127 | y |
| siRNA_b2_128 | n |
| siRNA_b2_129 | y |
| siRNA_b2_130 | y |
| siRNA_b2_131 | y |
| siRNA_b2_132 | y |
| siRNA_b2_133 | y |
| siRNA_b2_134 | y |
| siRNA_b2_135 | y |
| siRNA_b2_136 | y |
| siRNA_b2_137 | y |
| siRNA_b2_138 | y |
| siRNA_b2_139 | y |
| siRNA_b2_140 | y |
| siRNA_b2_141 | y |
| siRNA_b2_142 | y |
| siRNA_b2_143 | y |
| siRNA_b2_144 | y |
| siRNA_b2_145 | y |
| siRNA_b2_146 | y |
| siRNA_b2_147 | y |
| siRNA_b2_148 | y |
| siRNA_b2_149 | n |
| siRNA_b2_150 | n |
| siRNA_b2_151 | y |
| siRNA_b2_152 | y |
| siRNA_b2_153 | n |
| siRNA_b2_154 | y |
| siRNA_b2_155 | y |
| siRNA_b2_156 | y |
| siRNA_b2_157 | y |
| siRNA_b2_158 | y |
| siRNA_b2_159 | y |
| siRNA_b2_160 | n |
| siRNA_b2_161 | y |
| siRNA_b2_162 | y |
| siRNA_b2_163 | n |
| siRNA_b2_164 | n |
| siRNA_b2_165 | n |
| siRNA_b2_166 | n |
| siRNA_b2_167 | y |
| siRNA_b2_168 | y |
| siRNA_b2_169 | y |
| siRNA_b2_170 | n |
| siRNA_b2_171 | n |
| siRNA_b2_172 | y |
| siRNA_b2_173 | n |
| siRNA_b2_174 | n |
| siRNA_b2_175 | n |
| siRNA_b2_176 | n |
| siRNA_b2_177 | n |
| siRNA_b2_178 | n |
| siRNA_b2_179 | n |
| siRNA_b2_180 | y |

The normalized proteomic eigenvalues, signal pathwayomic eigenvalues, and core genomic eigenvalues were taken as input values, and the binarized cell survival indexes were taken as an output value into a Probabilistic Neural Network (PNN). PNN is a feedforward neural network based on density function estimation and Bayesian decision theory. It is often used for pattern classification. In this example, the PNN Learner (DDA) node in the KNIME® Analytics Platform software was used. The PNN model generated by this node is based on the Dynamic Decay Adjustment (DDA) algorithm, wherein the main adjustable parameters are Theta Minus and Theta Plus. In the preferred solution, Theta Minus may be set to 0.2 and Theta Plus may be set to 0.4.

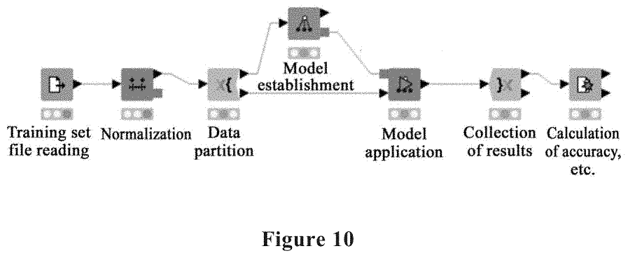

The model was evaluated by 10-fold cross validation, and the specific nodes and their connection order are shown in FIG. 10. The data set was divided into 10 parts (X-partitioner), 9 of which were used for training and 1 for verifying in turn. 10 results (X-aggregator) were collected and then the algorithm accuracy (Scorer) was calculated. The 10-fold cross validation was repeated 5 times and the average of the algorithm accuracy was taken. The accuracy of the above algorithm could reach 55.2%.

(2) Establishing a Machine Learning Model Through the Machine Learning Algorithm SVM

As described above, proteomic eigenvalues, signal pathwayomic eigenvalues, and core genomic eigenvalues were obtained for a specific siRNA. These data need to be normalized before being used as input values for machine learning algorithms. The data were mapped one-to-one to the interval 0-1 using the formula: (a value-minimum)/(maximum-minimum). The normalization of the above data could be achieved by the Normalizer node in the KNIME® Analytics Platform software. The results are the same as those reported in Table 16.

For the output value data of the machine learning algorithm, that is, the survival indexes of the cells in the presence of the siRNAs, they were binarized before being used as the output value data (for example, with a survival index of 0.75 as a boundary value, higher than or equal to 0.75 being set to y, and the rest being set to n). The results are the same as those reported in Table 17.

The normalized proteomic eigenvalues, signal pathwayomic eigenvalues, and core genomic eigenvalues were taken as input values, and the binarized cell survival indexes were taken as output values into the support vector machine algorithm (SVM). The SVM Learner node in the KNIME® Analytics Platform software was used, wherein the main adjustable parameter was the kernel and parameters, and the preferred setting thereof was RBF.

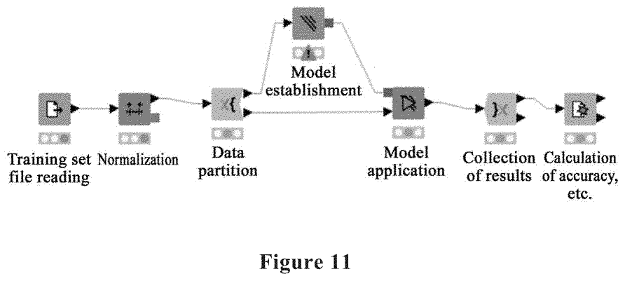

The model was evaluated using 10-fold cross validation. The specific nodes and their connection order are shown in FIG. 11. The data set was divided into 10 parts (X-partitioner), 9 of which were used for training and 1 for verifying in turn. 10 results (X-aggregator) were collected and the algorithm accuracy (Scorer) was calculated. The 10-fold cross validation was repeated 5 times and the average of the algorithm accuracy was taken. The accuracy of the above algorithm could reach 59.9%.

1. A method of establishing a machine learning model for predicting toxicity of an siRNA to a certain type of cells, comprising the following steps:
A) providing n siRNAs, wherein n≥2, and wherein the siRNAs are 19-29 bp in length;
B) separately obtaining an input value and an output value for establishing a machine learning model from each of the siRNAs;
wherein, the input value of any one of the n siRNAs is obtained as follows:
i) aligning a sequence of the siRNA with sequences of genomic mRNAs, respectively, and selecting one or more off-target genes located in the genomic mRNAs, which are complementary to the siRNA and the number of mismatched bases therebetween is less than or equal to 7;
ii) obtaining an off-target weight of each of the selected off-target genes regarding each complementary region of the off-target gene's mRNA to the siRNA sequence, independently, according to characteristic of the mismatched bases and secondary structure characteristic of the off-target gene's mRNA sequence;
iii) independently of ii) and unsequentially with ii), annotating each of the selected off-target genes using bioinformatics databases, and therefore obtaining omic weights of the off-target gene, including at least one selected from the group consisting of: protein interaction weight, signal pathway weight and core gene weight of the off-target gene; and
iv) calculating each omic eigenvalue based on the respective omic weights and the off-target weights of all the selected off-target genes, and using each of the eigenvalues as the input value;
and wherein, the output value of the siRNA is obtained as follows:
using the siRNA to conduct experiments in a certain type of cells to obtain a cell survival index in the presence of the siRNA, and using the cell survival index as the output value; and
C) establishing the machine learning model by calculating all the input values and the output values of the n siRNAs through a machine learning algorithm.

2. The method according to claim 1, wherein the characteristic of the mismatched bases comprises the number of the mismatched bases, and optionally, the position of the mismatched bases.

3. The method according to claim 1, wherein the secondary structural characteristic of the off-target gene's mRNA sequence is a probability of the mRNA itself not forming a secondary structure in the complementary region.

4. The method according to claim 3, wherein for each of the selected off-target genes, an interference rate of the siRNA on the expression level of the off-target gene's mRNA is calculated according to the characteristic of the mismatched bases, and then, a product of the interference rate and the probability of not forming the secondary structure is calculated to obtain the off-target weight of the off-target gene.

5. The method according to claim 3, wherein the probability of the mRNA of each off-target gene not forming a secondary structure is predicted using a software selected from the group consisting of: RNAPLFOLD, mfold or RNAstructure.

6. The method according to claim 1, wherein the omic eigenvalues include at least one selected from the group consisting of: a proteomic eigenvalue, a signal pathwayomic eigenvalue, and a core genomic eigenvalue; and wherein the proteomic eigenvalue, the signal pathwayomic eigenvalue and the core genomic eigenvalue are calculated according to the following a) to c), respectively:
a) calculating a product a′ of the off-target weight of each of the selected off-target genes and its protein interaction weight, and then calculating a sum of all the products a′ obtained for each of the selected off-target genes to generate a proteomic eigenvalue;
b) calculating a product b′ of the off-target weight of each of the selected off-target genes and its signal pathway weight, and then calculating a sum of all the products b′ obtained for each of the selected off-target genes to generate a signal pathwayomic eigenvalue;
c) calculating a product c′ of the off-target weight of each of the selected off-target genes and its core gene weight, and then calculating a sum of all the products c′ obtained for each of the selected off-target genes to generate a core genomic eigenvalue.

7. The method according to claim 1, wherein all the input values are normalized prior to establishing the machine learning model.

8. The method according to claim 1, wherein the machine learning algorithm comprises: a support vector machine, an artificial neural network, a decision tree, or a regression model.

9. The method according to claim 1, wherein in the step i), the selected off-target gene does not comprise such an off-target gene that a complementary region of its mRNA to the siRNA sequence is located only in its 5′ UTR.

10. The method according to claim 1, wherein in the step i), the selected off-target gene does not include a gene which is not expressed in the certain type of cells in a normal state.

11. (canceled)

12. A computer readable medium, wherein the computer readable medium can be used to establish the machine learning model on the basis of the method according to claim 1, and the computer readable medium comprises the following modules:
a sequence alignment module for performing the step i) in the method according to claim 1;
an off-target weight calculation module for performing the step ii) in the method according to claim 1;
an omic annotation module for performing the step iii) in the method according to claim 1;
an omic eigenvalue calculation module for performing the step iv) in the method according to claim 1; and
a machine learning algorithm calculation module for performing the step C) in the method according to claim 1.

13. A device for predicting toxicity of an siRNA to a certain type of cells, comprising:
1) an input unit for inputting a sequence of the siRNA to be tested;
2) a storage unit for storing a machine learning model established for a certain type of cells using the method according to claim 1;
3) an execution unit for executing the machine learning model on the sequence of the siRNA; and
4) an output unit for displaying a predicted result of the toxicity of the siRNA to the certain type of cells.

14. A method of predicting toxicity of an siRNA to a certain type of cells, comprising:
providing a sequence of the siRNA to be tested; and
inputting the sequence of the siRNA to a device for predicting toxicity of an siRNA to a certain type of cells, comprising:
1) an input unit for inputting a sequence of the siRNA to be tested;
2) a storage unit for storing a machine learning model established for a certain type of cells using the method according to claim 1;
3) an execution unit for executing the machine learning model on the sequence of the siRNA; and
4) an output unit for displaying a predicted result of the toxicity of the siRNA to the certain type of cells, and
allowing the device to execute the machine learning model established for the certain type of cells using the method according to claim 1, thereby obtaining result of the prediction of the toxicity of the siRNA to the certain type of cells.


SEQUENCE LISTING (398 sequences)

SEQ ID NO: 1 - DNA, 21 bp, artificial sequence
  siRNA sequence
ggaagccuauuuccgugaatt

SEQ ID NO: 2 - DNA, 21 bp, artificial sequence
  siRNA sequence
uucacggaaauaggcuucctt

SEQ ID NO: 3 - DNA, 21 bp, artificial sequence
  siRNA sequence
gacaaggauugugaaaugatt

SEQ ID NO: 4 - DNA, 21 bp, artificial sequence
  siRNA sequence
ucauuucacaauccuuguctt

SEQ ID NO: 5 - DNA, 21 bp, Artificial Sequence
  siRNA sequence
auggcuucuggcccaugaatt

SEQ ID NO: 6 - DNA, 21 bp, Artificial Sequence
  siRNA sequence
uucaugggccagaagccautt

SEQ ID NO: 7 - DNA, 21 bp, artificial sequence
  siRNA sequence
ccagacagguguuauggaatt

SEQ ID NO: 8 - DNA, 21 bp, artificial sequence
  siRNA sequence
uuccauaacaccugucuggtt

SEQ ID NO: 9 - DNA, 21 bp, artificial sequence
  siRNA sequence
ccagacagguguuauggautt

SEQ ID NO: 10 - DNA, 21 bp, artificial sequence
  siRNA sequence
auccauaacaccugucuggtt

SEQ ID NO: 11 - DNA, 21 bp, artificial sequence
  siRNA sequence
ccagacagguguuaugguutt

SEQ ID NO: 12 - DNA, 21 bp, artificial sequence
  siRNA sequence
aaccauaacaccugucuggtt

SEQ ID NO: 13 - DNA, 21 bp, artificial sequence
  siRNA sequence
ccagacagguguuaugcuutt

SEQ ID NO: 14 - DNA, 21 bp, artificial sequence
  siRNA sequence
aagcauaacaccugucuggtt

SEQ ID NO: 15 - DNA, 21 bp, artificial sequence
  siRNA sequence
ccagacagguguuauccuutt

SEQ ID NO: 16 - DNA, 21 bp, artificial sequence
  siRNA sequence
aaggauaacaccugucuggtt

SEQ ID NO: 17 - DNA, 21 bp, artificial sequence
  siRNA sequence
ccagacagguguuaaccuutt

SEQ ID NO: 18 - DNA, 21 bp, artificial sequence
  siRNA sequence
aagguuaacaccugucuggtt

SEQ ID NO: 19 - DNA, 21 bp, artificial sequence
  siRNA sequence
ccagacagguguuuaccuutt

SEQ ID NO: 20 - DNA, 21 bp, artificial sequence
  siRNA sequence
aagguaaacaccugucuggtt

SEQ ID NO: 21 - DNA, 21 bp, artificial sequence
  siRNA sequence
ccagacagguguauaccuutt

SEQ ID NO: 22 - DNA, 21 bp, artificial sequence
  siRNA sequence
aagguauacaccugucuggtt

SEQ ID NO: 23 - DNA, 21 bp, artificial sequence
  siRNA sequence
gcagacagguguuauggaatt

SEQ ID NO: 24 - DNA, 21 bp, artificial sequence
  siRNA sequence
uuccauaacaccugucugctt

SEQ ID NO: 25 - DNA, 21 bp, artificial sequence
  siRNA sequence
ggagacagguguuauggaatt

SEQ ID NO: 26 - DNA, 21 bp, artificial sequence
  siRNA sequence
uuccauaacaccugucucctt

SEQ ID NO: 27 - DNA, 21 bp, artificial sequence
  siRNA sequence
ggugacagguguuauggaatt

SEQ ID NO: 28 - DNA, 21 bp, artificial sequence
  siRNA sequence
uuccauaacaccugucacctt

SEQ ID NO: 29 - DNA, 21 bp, artificial sequence
  siRNA sequence
ggucacagguguuauggaatt

SEQ ID NO: 30 - DNA, 21 bp, artificial sequence
  siRNA sequence
uuccauaacaccugugacctt

SEQ ID NO: 31 - DNA, 21 bp, artificial sequence
  siRNA sequence
ggucucagguguuauggaatt

SEQ ID NO: 32 - DNA, 21 bp, artificial sequence
  siRNA sequence
uuccauaacaccugagacctt

SEQ ID NO: 33 - DNA, 21 bp, artificial sequence
  siRNA sequence
ggucugagguguuauggaatt

SEQ ID NO: 34 - DNA, 21 bp, artificial sequence
  siRNA sequence
uuccauaacaccucagacctt

SEQ ID NO: 35 - DNA, 21 bp, artificial sequence
  siRNA sequence
ggucugugguguuauggaatt

SEQ ID NO: 36 - DNA, 21 bp, artificial sequence
  siRNA sequence
uuccauaacaccacagacctt

SEQ ID NO: 37 - DNA, 21 bp, artificial sequence
  siRNA sequence
ccagacagcacuuauggaatt

SEQ ID NO: 38 - DNA, 21 bp, artificial sequence
  siRNA sequence
uuccauaagugcugucuggtt

SEQ ID NO: 39 - RNA, 19 nt, artificial sequence
  siRNA sequence
auauuccuuaagggcuucg

SEQ ID NO: 40 - RNA, 19 nt, artificial sequence
  siRNA sequence
cgaagcccuuaaggaauau

SEQ ID NO: 41 - RNA, 19 nt, artificial sequence
  siRNA sequence
augauccagacugcaaugc

SEQ ID NO: 42 - RNA, 19 nt, artificial sequence
  siRNA sequence
gcauugcagucuggaucau

SEQ ID NO: 43 - RNA, 19 nt, artificial sequence
  siRNA sequence
aguacaaccaaggguuucc

SEQ ID NO: 44 - RNA, 19 nt, artificial sequence
  siRNA sequence
ggaaacccuugguuguacu

SEQ ID NO: 45 - RNA, 19 nt, artificial sequence
  siRNA sequence
agaaagacccuucaauucg

SEQ ID NO: 46 - RNA, 19 nt, artificial sequence
  siRNA sequence
cgaauugaagggucuuucu

SEQ ID NO: 47 - RNA, 19 nt, artificial sequence
  siRNA sequence
aauaaaguuggcagagucc

SEQ ID NO: 48 - RNA, 19 nt, artificial sequence
  siRNA sequence
ggacucugccaacuuuauu

SEQ ID NO: 49 - RNA, 19 nt, artificial sequence
  siRNA sequence
ucugaagggagagaaagag

SEQ ID NO: 50 - RNA, 19 nt, artificial sequence
  siRNA sequence
cucuuucucucccuucaga

SEQ ID NO: 51 - RNA, 19 nt, artificial sequence
  siRNA sequence
uaagauucugaagggagag

SEQ ID NO: 52 - RNA, 19 nt, artificial sequence
  siRNA sequence
cucucccuucagaaucuua

SEQ ID NO: 53 - RNA, 19 nt, artificial sequence
  siRNA sequence
ucuucuaagauccaaagcc

SEQ ID NO: 54 - RNA, 19 nt, artificial sequence
  siRNA sequence
ggcuuuggaucuuagaaga

SEQ ID NO: 55 - RNA, 19 nt, artificial sequence
  siRNA sequence
uaauagggaugggcucaac

SEQ ID NO: 56 - RNA, 19 nt, artificial sequence
  siRNA sequence
guugagcccaucccuauua

SEQ ID NO: 57 - RNA, 19 nt, artificial sequence
  siRNA sequence
uuucugggaaagcuuguag

SEQ ID NO: 58 - RNA, 19 nt, artificial sequence
  siRNA sequence
cuacaagcuuucccagaaa

SEQ ID NO: 59 - RNA, 19 nt, artificial sequence
  siRNA sequence
uauacuugaggccacaguc

SEQ ID NO: 60 - RNA, 19 nt, artificial sequence
  siRNA sequence
gacuguggccucaaguaua

SEQ ID NO: 61 - RNA, 19 nt, artificial sequence
  siRNA sequence
ucaaaugaacgcccaaugc

SEQ ID NO: 62 - RNA, 19 nt, artificial sequence
  siRNA sequence
gcauugggcguucauuuga

SEQ ID NO: 63 - RNA, 19 nt, artificial sequence
  siRNA sequence
uaacuuucagcuggucauc

SEQ ID NO: 64 - RNA, 19 nt, artificial sequence
  siRNA sequence
gaugaccagcugaaaguua

SEQ ID NO: 65 - RNA, 19 nt, artificial sequence
  siRNA sequence
ucaguguagaagucagcug

SEQ ID NO: 66 - RNA, 19 nt, artificial sequence
  siRNA sequence
cagcugacuucuacacuga

SEQ ID NO: 67 - RNA, 19 nt, artificial sequence
  siRNA sequence
ugagacaucugauccuugg

SEQ ID NO: 68 - RNA, 19 nt, artificial sequence
  siRNA sequence
ccaaggaucagaugucuca

SEQ ID NO: 69 - RNA, 19 nt, artificial sequence
  siRNA sequence
auuuuggucugacugcuug

SEQ ID NO: 70 - RNA, 19 nt, artificial sequence
  siRNA sequence
caagcagucagaccaaaau

SEQ ID NO: 71 - RNA, 19 nt, artificial sequence
  siRNA sequence
aauggagacagucauguac

SEQ ID NO: 72 - RNA, 19 nt, artificial sequence
  siRNA sequence
guacaugacugucuccauu

SEQ ID NO: 73 - RNA, 19 nt, artificial sequence
  siRNA sequence
auaaacauggcagugacac

SEQ ID NO: 74 - RNA, 19 nt, artificial sequence
  siRNA sequence
gugucacugccauguuuau

SEQ ID NO: 75 - RNA, 19 nt, artificial sequence
  siRNA sequence
uuucuggaggguacauuuc

SEQ ID NO: 76 - RNA, 19 nt, artificial sequence
  siRNA sequence
gaaauguacccuccagaaa

SEQ ID NO: 77 - RNA, 19 nt, artificial sequence
  siRNA sequence
uguccauucaccauuaucc

SEQ ID NO: 78 - RNA, 19 nt, artificial sequence
  siRNA sequence
ggauaauggugaauggaca

SEQ ID NO: 79 - RNA, 19 nt, artificial sequence
  siRNA sequence
uuugaaguaggacaccgag

SEQ ID NO: 80 - RNA, 19 nt, artificial sequence
  siRNA sequence
cucgguguccuacuucaaa

SEQ ID NO: 81 - RNA, 19 nt, artificial sequence
  siRNA sequence
uguagaugcacagcuucuc

SEQ ID NO: 82 - RNA, 19 nt, artificial sequence
  siRNA sequence
gagaagcugugcaucuaca

SEQ ID NO: 83 - RNA, 19 nt, artificial sequence
  siRNA sequence
uguucaaugaaaucgugcg

SEQ ID NO: 84 - RNA, 19 nt, artificial sequence
  siRNA sequence
cgcacgauuucauugaaca

SEQ ID NO: 85 - RNA, 19 nt, artificial sequence
  siRNA sequence
ucacacuugaucacucugg

SEQ ID NO: 86 - RNA, 19 nt, artificial sequence
  siRNA sequence
ccagagugaucaaguguga

SEQ ID NO: 87 - RNA, 19 nt, artificial sequence
  siRNA sequence
ucugguaucaaaaugcucc

SEQ ID NO: 88 - RNA, 19 nt, artificial sequence
  siRNA sequence
ggagcauuuugauaccaga

SEQ ID NO: 89 - RNA, 19 nt, artificial sequence
  siRNA sequence
auuaggaugguuaagcucc

SEQ ID NO: 90 - RNA, 19 nt, artificial sequence
  siRNA sequence
ggagcuuaaccauccuaau

SEQ ID NO: 91 - RNA, 19 nt, artificial sequence
  siRNA sequence
uguaaguacgaacagggac

SEQ ID NO: 92 - RNA, 19 nt, artificial sequence
  siRNA sequence
gucccuguucguacuuaca

SEQ ID NO: 93 - RNA, 19 nt, artificial sequence
  siRNA sequence
aauauuugcagcccaggag

SEQ ID NO: 94 - RNA, 19 nt, artificial sequence
  siRNA sequence
cuccugggcugcaaauauu

SEQ ID NO: 95 - RNA, 19 nt, artificial sequence
  siRNA sequence
aaucucagaaucuccaggg

SEQ ID NO: 96 - RNA, 19 nt, artificial sequence
  siRNA sequence
cccuggagauucugagauu

SEQ ID NO: 97 - RNA, 19 nt, artificial sequence
  siRNA sequence
uuacuaaaaucuugccggg

SEQ ID NO: 98 - RNA, 19 nt, artificial sequence
  siRNA sequence
cccggcaagauuuuaguaa

SEQ ID NO: 99 - RNA, 19 nt, artificial sequence
  siRNA sequence
uuagaaggaggaacuccag

SEQ ID NO: 100 - RNA, 19 nt, artificial sequence
  siRNA sequence
cuggaguuccuccuucuaa

SEQ ID NO: 101 - RNA, 19 nt, artificial sequence
  siRNA sequence
uaauuccaggccaacaaac

SEQ ID NO: 102 - RNA, 19 nt, artificial sequence
  siRNA sequence
guuuguuggccuggaauua

SEQ ID NO: 103 - RNA, 19 nt, artificial sequence
  siRNA sequence
auuccauucagcacuuugc

SEQ ID NO: 104 - RNA, 19 nt, artificial sequence
  siRNA sequence
gcaaagugcugaauggaau

SEQ ID NO: 105 - RNA, 19 nt, artificial sequence
  siRNA sequence
uaccuguuuauucaguggc

SEQ ID NO: 106 - RNA, 19 nt, artificial sequence
  siRNA sequence
gccacugaauaaacaggua

SEQ ID NO: 107 - RNA, 19 nt, artificial sequence
  siRNA sequence
aauucaguacucucucugg

SEQ ID NO: 108 - RNA, 19 nt, artificial sequence
  siRNA sequence
ccagagagaguacugaauu

SEQ ID NO: 109 - RNA, 19 nt, artificial sequence
  siRNA sequence
uaguucuugggaaugaagc

SEQ ID NO: 110 - RNA, 19 nt, artificial sequence
  siRNA sequence
gcuucauucccaagaacua

SEQ ID NO: 111 - RNA, 19 nt, artificial sequence
  siRNA sequence
uuuugccaaaaaaccacgg

SEQ ID NO: 112 - RNA, 19 nt, artificial sequence
  siRNA sequence
ccgugguuuuuuggcaaaa

SEQ ID NO: 113 - RNA, 19 nt, artificial sequence
  siRNA sequence
aaacuugacagagagggag

SEQ ID NO: 114 - RNA, 19 nt, artificial sequence
  siRNA sequence
cucccucucugucaaguuu

SEQ ID NO: 115 - RNA, 19 nt, artificial sequence
  siRNA sequence
aauaucugcugguuucugg

SEQ ID NO: 116 - RNA, 19 nt, artificial sequence
  siRNA sequence
ccagaaaccagcagauauu

SEQ ID NO: 117 - RNA, 19 nt, artificial sequence
  siRNA sequence
ugaguuauccaugacaugg

SEQ ID NO: 118 - RNA, 19 nt, artificial sequence
  siRNA sequence
ccaugucauggauaacuca

SEQ ID NO: 119 - RNA, 19 nt, artificial sequence
  siRNA sequence
aaagaaggguugcacuugc

SEQ ID NO: 120 - RNA, 19 nt, artificial sequence
  siRNA sequence
gcaagugcaacccuucuuu

SEQ ID NO: 121 - RNA, 19 nt, artificial sequence
  siRNA sequence
uaaggaucaacaaggcucc

SEQ ID NO: 122 - RNA, 19 nt, artificial sequence
  siRNA sequence
ggagccuuguugauccuua

SEQ ID NO: 123 - RNA, 19 nt, artificial sequence
  siRNA sequence
uuuuguuccgaagcccaug

SEQ ID NO: 124 - RNA, 19 nt, artificial sequence
  siRNA sequence
caugggcuucggaacaaaa

SEQ ID NO: 125 - RNA, 19 nt, artificial sequence
  siRNA sequence
uaucugugaaggcagaagg

SEQ ID NO: 126 - RNA, 19 nt, artificial sequence
  siRNA sequence
ccuucugccuucacagaua

SEQ ID NO: 127 - RNA, 19 nt, artificial sequence
  siRNA sequence
uuaugggcgaaguccuuug

SEQ ID NO: 128 - RNA, 19 nt, artificial sequence
  siRNA sequence
caaaggacuucgcccauaa

SEQ ID NO: 129 - RNA, 19 nt, artificial sequence
  siRNA sequence
aaauucaccagaaggcauc

SEQ ID NO: 130 - RNA, 19 nt, artificial sequence
  siRNA sequence
gaugccuucuggugaauuu

SEQ ID NO: 131 - RNA, 19 nt, artificial sequence
  siRNA sequence
uuuccaaguucuccacuug

SEQ ID NO: 132 - RNA, 19 nt, artificial sequence
  siRNA sequence
caaguggagaacuuggaaa

SEQ ID NO: 133 - RNA, 19 nt, artificial sequence
  siRNA sequence
uaugguaacagcuuccucc

SEQ ID NO: 134 - RNA, 19 nt, artificial sequence
  siRNA sequence
ggaggaagcuguuaccaua

SEQ ID NO: 135 - RNA, 19 nt, artificial sequence
  siRNA sequence
auacugagugucaccguug

SEQ ID NO: 136 - RNA, 19 nt, artificial sequence
  siRNA sequence
caacggugacacucaguau

SEQ ID NO: 137 - RNA, 19 nt, artificial sequence
  siRNA sequence
ucuucauccucgaucuugg

SEQ ID NO: 138 - RNA, 19 nt, artificial sequence
  siRNA sequence
ccaagaucgaggaugaaga

SEQ ID NO: 139 - RNA, 19 nt, artificial sequence
  siRNA sequence
uguuuccugcacauguuug

SEQ ID NO: 140 - RNA, 19 nt, artificial sequence
  siRNA sequence
caaacaugugcaggaaaca

SEQ ID NO: 141 - RNA, 19 nt, artificial sequence
  siRNA sequence
uuccacaccgaacuuguug

SEQ ID NO: 142 - RNA, 19 nt, artificial sequence
  siRNA sequence
caacaaguucgguguggaa

SEQ ID NO: 143 - RNA, 19 nt, artificial sequence
  siRNA sequence
uuaacgugcuuccauuccg

SEQ ID NO: 144 - RNA, 19 nt, artificial sequence
  siRNA sequence
cggaauggaagcacguuaa

SEQ ID NO: 145 - RNA, 19 nt, artificial sequence
  siRNA sequence
uaguaugacccucgaugag

SEQ ID NO: 146 - RNA, 19 nt, artificial sequence
  siRNA sequence
cucaucgagggucauacua

SEQ ID NO: 147 - RNA, 19 nt, artificial sequence
  siRNA sequence
ucauaguagacauucaccc

SEQ ID NO: 148 - RNA, 19 nt, artificial sequence
  siRNA sequence
gggugaaugucuacuauga

SEQ ID NO: 149 - RNA, 19 nt, artificial sequence
  siRNA sequence
aguaacuggacaucgaacc

SEQ ID NO: 150 - RNA, 19 nt, artificial sequence
  siRNA sequence
gguucgauguccaguuacu

SEQ ID NO: 151 - RNA, 19 nt, artificial sequence
  siRNA sequence
agaauggugaugcguucac

SEQ ID NO: 152 - RNA, 19 nt, artificial sequence
  siRNA sequence
gugaacgcaucaccauucu

SEQ ID NO: 153 - RNA, 19 nt, artificial sequence
  siRNA sequence
uguaucuauagauggcgag

SEQ ID NO: 154 - RNA, 19 nt, artificial sequence
  siRNA sequence
cucgccaucuauagauaca

SEQ ID NO: 155 - RNA, 19 nt, artificial sequence
  siRNA sequence
uuuggagcacugaaaaucg

SEQ ID NO: 156 - RNA, 19 nt, artificial sequence
  siRNA sequence
cgauuuucagugcuccaaa

SEQ ID NO: 157 - RNA, 19 nt, artificial sequence
  siRNA sequence
uagaguaucgucaaguucc

SEQ ID NO: 158 - RNA, 19 nt, artificial sequence
  siRNA sequence
ggaacuugacgauacucua

SEQ ID NO: 159 - RNA, 19 nt, artificial sequence
  siRNA sequence
uaaagcggccauugucuug

SEQ ID NO: 160 - RNA, 19 nt, artificial sequence
  siRNA sequence
caagacaauggccgcuuua

SEQ ID NO: 161 - RNA, 19 nt, artificial sequence
  siRNA sequence
ugaaucacagucucuccug

SEQ ID NO: 162 - RNA, 19 nt, artificial sequence
  siRNA sequence
caggagagacugugauuca

SEQ ID NO: 163 - RNA, 19 nt, artificial sequence
  siRNA sequence
uucuucuauagcugucucg

SEQ ID NO: 164 - RNA, 19 nt, artificial sequence
  siRNA sequence
cgagacagcuauagaagaa

SEQ ID NO: 165 - RNA, 19 nt, artificial sequence
  siRNA sequence
uaagacguucccacuuguc

SEQ ID NO: 166 - RNA, 19 nt, artificial sequence
  siRNA sequence
gacaagugggaacgucuua

SEQ ID NO: 167 - RNA, 19 nt, artificial sequence
  siRNA sequence
aaaacuguuguacugcugg

SEQ ID NO: 168 - RNA, 19 nt, artificial sequence
  siRNA sequence
ccagcaguacaacaguuuu

SEQ ID NO: 169 - RNA, 19 nt, artificial sequence
  siRNA sequence
uuacuuugugacuguccac

SEQ ID NO: 170 - RNA, 19 nt, artificial sequence
  siRNA sequence
guggacagucacaaaguaa

SEQ ID NO: 171 - RNA, 19 nt, artificial sequence
  siRNA sequence
uauaaucgcucuucaccug

SEQ ID NO: 172 - RNA, 19 nt, artificial sequence
  siRNA sequence
caggugaagagcgauuaua

SEQ ID NO: 173 - RNA, 19 nt, artificial sequence
  siRNA sequence
uuaguguuuuggccuugac

SEQ ID NO: 174 - RNA, 19 nt, artificial sequence
  siRNA sequence
gucaaggccaaaacacuaa

SEQ ID NO: 175 - RNA, 19 nt, artificial sequence
  siRNA sequence
uugguauugauggcaaagc

SEQ ID NO: 176 - RNA, 19 nt, artificial sequence
  siRNA sequence
gcuuugccaucaauaccaa

SEQ ID NO: 177 - RNA, 19 nt, artificial sequence
  siRNA sequence
aaucauuugaggacaccag

SEQ ID NO: 178 - RNA, 19 nt, artificial sequence
  siRNA sequence
cugguguccucaaaugauu

SEQ ID NO: 179 - RNA, 19 nt, artificial sequence
  siRNA sequence
uguaauacuggaccaacuc

SEQ ID NO: 180 - DNA, 21 bp, artificial sequence
  siRNA sequence
gaguugguccaguauuacatt

SEQ ID NO: 181 - RNA, 19 nt, artificial sequence
  siRNA sequence
aagaaucaaaccguucucc

SEQ ID NO: 182 - RNA, 19 nt, artificial sequence
  siRNA sequence
ggagaacgguuugauucuu

SEQ ID NO: 183 - RNA, 19 nt, artificial sequence
  siRNA sequence
uguaaucugaaacaggcuc

SEQ ID NO: 184 - RNA, 19 nt, artificial sequence
  siRNA sequence
gagccuguuucagauuaca

SEQ ID NO: 185 - RNA, 19 nt, artificial sequence
  siRNA sequence
uuguguggcaauguaacuc

SEQ ID NO: 186 - RNA, 19 nt, artificial sequence
  siRNA sequence
gaguuacauugccacacaa

SEQ ID NO: 187 - RNA, 19 nt, artificial sequence
  siRNA sequence
uuucuuggaacaccauccg

SEQ ID NO: 188 - RNA, 19 nt, artificial sequence
  siRNA sequence
cggaugguguuccaagaaa

SEQ ID NO: 189 - RNA, 19 nt, artificial sequence
  siRNA sequence
uuguucggcaagaaaacac

SEQ ID NO: 190 - RNA, 19 nt, artificial sequence
  siRNA sequence
guguuuucuugccgaacaa

SEQ ID NO: 191 - RNA, 19 nt, artificial sequence
  siRNA sequence
uuucauaaggcagucaugc

SEQ ID NO: 192 - RNA, 19 nt, artificial sequence
  siRNA sequence
gcaugacugccuuaugaaa

SEQ ID NO: 193 - RNA, 19 nt, artificial sequence
  siRNA sequence
uuuaccuuuguguucgugg

SEQ ID NO: 194 - RNA, 19 nt, artificial sequence
  siRNA sequence
ccacgaacacaaagguaaa

SEQ ID NO: 195 - RNA, 19 nt, artificial sequence
  siRNA sequence
uugagcaggaauuucugac

SEQ ID NO: 196 - RNA, 19 nt, artificial sequence
  siRNA sequence
gucagaaauuccugcucaa

SEQ ID NO: 197 - RNA, 19 nt, artificial sequence
  siRNA sequence
ucugauguuacuccagucc

SEQ ID NO: 198 - RNA, 19 nt, artificial sequence
  siRNA sequence
ggacuggaguaacaucaga

SEQ ID NO: 199 - RNA, 19 nt, artificial sequence
  siRNA sequence
aaaguuuggcugcucuuuc

SEQ ID NO: 200 - RNA, 19 nt, artificial sequence
  siRNA sequence
gaaagagcagccaaacuuu

SEQ ID NO: 201 - RNA, 19 nt, artificial sequence
  siRNA sequence
auuacuacuaugcugaccc

SEQ ID NO: 202 - RNA, 19 nt, artificial sequence
  siRNA sequence
gggucagcauaguaguaau

SEQ ID NO: 203 - RNA, 19 nt, artificial sequence
  siRNA sequence
uuuacauugccaaucccac

SEQ ID NO: 204 - RNA, 19 nt, artificial sequence
  siRNA sequence
gugggauuggcaauguaaa

SEQ ID NO: 205 - RNA, 19 nt, artificial sequence
  siRNA sequence
acuuaaaagaggcaggagc

SEQ ID NO: 206 - RNA, 19 nt, artificial sequence
  siRNA sequence
gcuccugccucuuuuaagu

SEQ ID NO: 207 - RNA, 19 nt, artificial sequence
  siRNA sequence
uuuagaggcaucacaagcc

SEQ ID NO: 208 - RNA, 19 nt, artificial sequence
  siRNA sequence
ggcuugugaugccucuaaa

SEQ ID NO: 209 - RNA, 19 nt, artificial sequence
  siRNA sequence
uuuauaaccuaggaccucc

SEQ ID NO: 210 - RNA, 19 nt, artificial sequence
  siRNA sequence
ggagguccuagguuauaaa

SEQ ID NO: 211 - RNA, 19 nt, artificial sequence
  siRNA sequence
uaaguuuguucuccugagg

SEQ ID NO: 212 - RNA, 19 nt, artificial sequence
  siRNA sequence
ccucaggagaacaaacuua

SEQ ID NO: 213 - RNA, 19 nt, artificial sequence
  siRNA sequence
uauucugcauugcuagcac

SEQ ID NO: 214 - RNA, 19 nt, artificial sequence
  siRNA sequence
gugcuagcaaugcagaaua

SEQ ID NO: 215 - RNA, 19 nt, artificial sequence
  siRNA sequence
auuuucuucuggcgacuug

SEQ ID NO: 216 - RNA, 19 nt, artificial sequence
  siRNA sequence
caagucgccagaagaaaau

SEQ ID NO: 217 - RNA, 19 nt, artificial sequence
  siRNA sequence
uucuguuucacuuucaggg

SEQ ID NO: 218 - RNA, 19 nt, artificial sequence
  siRNA sequence
cccugaaagugaaacagaa

SEQ ID NO: 219 - RNA, 19 nt, artificial sequence
  siRNA sequence
uuauauucggcguuucggg

SEQ ID NO: 220 - RNA, 19 nt, artificial sequence
  siRNA sequence
cccgaaacgccgaauauaa

SEQ ID NO: 221 - RNA, 19 nt, artificial sequence
  siRNA sequence
aaaaucagugccgugguuc

SEQ ID NO: 222 - RNA, 19 nt, artificial sequence
  siRNA sequence
gaaccacggcacugauuuu

SEQ ID NO: 223 - RNA, 19 nt, artificial sequence
  siRNA sequence
aaauuguuggugggugagc

SEQ ID NO: 224 - RNA, 19 nt, artificial sequence
  siRNA sequence
gcucacccaccaacaauuu

SEQ ID NO: 225 - RNA, 19 nt, artificial sequence
  siRNA sequence
ucaacauccaucuucucac

SEQ ID NO: 226 - RNA, 19 nt, artificial sequence
  siRNA sequence
gugagaagauggauguuga

SEQ ID NO: 227 - RNA, 19 nt, artificial sequence
  siRNA sequence
auaaauaaaugggcagcgc

SEQ ID NO: 228 - RNA, 19 nt, artificial sequence
  siRNA sequence
gcgcugcccauuuauuuau

SEQ ID NO: 229 - RNA, 19 nt, artificial sequence
  siRNA sequence
agccucugucccagugccc

SEQ ID NO: 230 - RNA, 19 nt, artificial sequence
  siRNA sequence
gggcacugggacagaggcu

SEQ ID NO: 231 - RNA, 19 nt, artificial sequence
  siRNA sequence
ucagccucugucccagugc

SEQ ID NO: 232 - RNA, 19 nt, artificial sequence
  siRNA sequence
gcacugggacagaggcuga

SEQ ID NO: 233 - RNA, 19 nt, artificial sequence
  siRNA sequence
uuucucaaacucagccucu

SEQ ID NO: 234 - RNA, 19 nt, artificial sequence
  siRNA sequence
agaggcugaguuugagaaa

SEQ ID NO: 235 - RNA, 19 nt, artificial sequence
  siRNA sequence
agcuuucucaaacucagcc

SEQ ID NO: 236 - RNA, 19 nt, artificial sequence
  siRNA sequence
ggcugaguuugagaaagcu

SEQ ID NO: 237 - RNA, 19 nt, artificial sequence
  siRNA sequence
uccucauccgauggcuugg

SEQ ID NO: 238 - RNA, 19 nt, artificial sequence
  siRNA sequence
ccaagccaucggaugagga

SEQ ID NO: 239 - RNA, 19 nt, artificial sequence
  siRNA sequence
ucaaucuugcuuguuugac

SEQ ID NO: 240 - RNA, 19 nt, artificial sequence
  siRNA sequence
gucaaacaagcaagauuga

SEQ ID NO: 241 - RNA, 19 nt, artificial sequence
  siRNA sequence
ucucaaucuugcuuguuug

SEQ ID NO: 242 - RNA, 19 nt, artificial sequence
  siRNA sequence
caaacaagcaagauugaga

SEQ ID NO: 243 - RNA, 19 nt, artificial sequence
  siRNA sequence
uaauccaugucagauucag

SEQ ID NO: 244 - RNA, 19 nt, artificial sequence
  siRNA sequence
cugaaucugacauggauua

SEQ ID NO: 245 - RNA, 19 nt, artificial sequence
  siRNA sequence
aauuucggaaggaauagac

SEQ ID NO: 246 - RNA, 19 nt, artificial sequence
  siRNA sequence
gucuauuccuuccgaaauu

SEQ ID NO: 247 - RNA, 19 nt, artificial sequence
  siRNA sequence
uugaauuugccuuugaacc

SEQ ID NO: 248 - RNA, 19 nt, artificial sequence
  siRNA sequence
gguucaaaggcaaauucaa

SEQ ID NO: 249 - RNA, 19 nt, artificial sequence
  siRNA sequence
ugaaaucacagcaucguug

SEQ ID NO: 250 - RNA, 19 nt, artificial sequence
  siRNA sequence
caacgaugcugugauuuca

SEQ ID NO: 251 - RNA, 19 nt, artificial sequence
  siRNA sequence
auuuacuccagaaagguuc

SEQ ID NO: 252 - RNA, 19 nt, artificial sequence
  siRNA sequence
gaaccuuucuggaguaaau

SEQ ID NO: 253 - RNA, 19 nt, artificial sequence
  siRNA sequence
uuaccauagcguuuguuug

SEQ ID NO: 254 - RNA, 19 nt, artificial sequence
  siRNA sequence
caaacaaacgcuaugguaa

SEQ ID NO: 255 - RNA, 19 nt, artificial sequence
  siRNA sequence
auuucuucugucauugucc

SEQ ID NO: 256 - RNA, 19 nt, artificial sequence
  siRNA sequence
ggacaaugacagaagaaau

SEQ ID NO: 257 - RNA, 19 nt, artificial sequence
  siRNA sequence
uagaauguggcgauacauc

SEQ ID NO: 258 - RNA, 19 nt, artificial sequence
  siRNA sequence
gauguaucgccacauucua

SEQ ID NO: 259 - RNA, 19 nt, artificial sequence
  siRNA sequence
ugaaucaucccauuguucc

SEQ ID NO: 260 - RNA, 19 nt, artificial sequence
  siRNA sequence
ggaacaaugggaugauuca

SEQ ID NO: 261 - RNA, 19 nt, artificial sequence
  siRNA sequence
uauaacuguggcuuaacgc

SEQ ID NO: 262 - RNA, 19 nt, artificial sequence
  siRNA sequence
gcguuaagccacaguuaua

SEQ ID NO: 263 - RNA, 19 nt, artificial sequence
  siRNA sequence
auucugaugcgaugguuug

SEQ ID NO: 264 - RNA, 19 nt, artificial sequence
  siRNA sequence
caaaccaucgcaucagaau

SEQ ID NO: 265 - RNA, 19 nt, artificial sequence
  siRNA sequence
auucucaagacucguaaug

SEQ ID NO: 266 - RNA, 19 nt, artificial sequence
  siRNA sequence
cauuacgagucuugagaau

SEQ ID NO: 267 - RNA, 19 nt, artificial sequence
  siRNA sequence
ucauaaacuggcuuuagac

SEQ ID NO: 268 - RNA, 19 nt, artificial sequence
  siRNA sequence
gucuaaagccaguuuauga

SEQ ID NO: 269 - RNA, 19 nt, artificial sequence
  siRNA sequence
aaugauguccaaugaguug

SEQ ID NO: 270 - RNA, 19 nt, artificial sequence
  siRNA sequence
caacucauuggacaucauu

SEQ ID NO: 271 - RNA, 19 nt, artificial sequence
  siRNA sequence
ugaauuagggcacauugag

SEQ ID NO: 272 - RNA, 19 nt, artificial sequence
  siRNA sequence
cucaaugugcccuaauuca

SEQ ID NO: 273 - RNA, 19 nt, artificial sequence
  siRNA sequence
uauuauucgccucuuucgg

SEQ ID NO: 274 - RNA, 19 nt, artificial sequence
  siRNA sequence
ccgaaagaggcgaauaaua

SEQ ID NO: 275 - RNA, 19 nt, artificial sequence
  siRNA sequence
uauaguucagcaguugaag

SEQ ID NO: 276 - RNA, 19 nt, artificial sequence
  siRNA sequence
cuucaacugcugaacuaua

SEQ ID NO: 277 - RNA, 19 nt, artificial sequence
  siRNA sequence
ucacuaaccuguaaugugc

SEQ ID NO: 278 - RNA, 19 nt, artificial sequence
  siRNA sequence
gcacauuacagguuaguga

SEQ ID NO: 279 - RNA, 19 nt, artificial sequence
  siRNA sequence
uuauggaaggcaaagucuc

SEQ ID NO: 280 - RNA, 19 nt, artificial sequence
  siRNA sequence
gagacuuugccuuccauaa

SEQ ID NO: 281 - RNA, 19 nt, artificial sequence
  siRNA sequence
ugauacaacugugaaagac

SEQ ID NO: 282 - RNA, 19 nt, artificial sequence
  siRNA sequence
gucuuucacaguuguauca

SEQ ID NO: 283 - RNA, 19 nt, artificial sequence
  siRNA sequence
aaauuaggguugcauuugg

SEQ ID NO: 284 - RNA, 19 nt, artificial sequence
  siRNA sequence
ccaaaugcaacccuaauuu

SEQ ID NO: 285 - RNA, 19 nt, artificial sequence
  siRNA sequence
uaaaccaucuugauugugc

SEQ ID NO: 286 - RNA, 19 nt, artificial sequence
  siRNA sequence
gcacaaucaagaugguuua

SEQ ID NO: 287 - RNA, 19 nt, artificial sequence
  siRNA sequence
uuauaacgccuguaacucc

SEQ ID NO: 288 - RNA, 19 nt, artificial sequence
  siRNA sequence
ggaguuacaggcguuauaa

SEQ ID NO: 289 - RNA, 19 nt, artificial sequence
  siRNA sequence
auuauaacgccuguaacuc

SEQ ID NO: 290 - RNA, 19 nt, artificial sequence
  siRNA sequence
gaguuacaggcguuauaau

SEQ ID NO: 291 - RNA, 19 nt, artificial sequence
  siRNA sequence
agaauaaagcgauaacugc

SEQ ID NO: 292 - RNA, 19 nt, artificial sequence
  siRNA sequence
gcaguuaucgcuuuauucu

SEQ ID NO: 293 - RNA, 19 nt, artificial sequence
  siRNA sequence
auuaguaggaguaauuccc

SEQ ID NO: 294 - RNA, 19 nt, artificial sequence
  siRNA sequence
gggaauuacuccuacuaau

SEQ ID NO: 295 - RNA, 19 nt, artificial sequence
  siRNA sequence
acuuucacacgguaacugg

SEQ ID NO: 296 - RNA, 19 nt, artificial sequence
  siRNA sequence
ccaguuaccgugugaaagu

SEQ ID NO: 297 - RNA, 19 nt, artificial sequence
  siRNA sequence
auugugaucaaguagaagg

SEQ ID NO: 298 - RNA, 19 nt, artificial sequence
  siRNA sequence
ccuucuacuugaucacaau

SEQ ID NO: 299 - RNA, 19 nt, artificial sequence
  siRNA sequence
uauauuagggcaaucaugc

SEQ ID NO: 300 - RNA, 19 nt, artificial sequence
  siRNA sequence
gcaugauugcccuaauaua

SEQ ID NO: 301 - RNA, 19 nt, artificial sequence
  siRNA sequence
uacaagaaucacuuugugc

SEQ ID NO: 302 - RNA, 19 nt, artificial sequence
  siRNA sequence
gcacaaagugauucuugua

SEQ ID NO: 303 - RNA, 19 nt, artificial sequence
  siRNA sequence
aaagugauguucguuguag

SEQ ID NO: 304 - RNA, 19 nt, artificial sequence
  siRNA sequence
cuacaacgaacaucacuuu

SEQ ID NO: 305 - RNA, 19 nt, artificial sequence
  siRNA sequence
auauuggaucgaaucaacg

SEQ ID NO: 306 - RNA, 19 nt, artificial sequence
  siRNA sequence
cguugauucgauccaauau

SEQ ID NO: 307 - RNA, 19 nt, artificial sequence
  siRNA sequence
uuguuugagggauucugag

SEQ ID NO: 308 - RNA, 19 nt, artificial sequence
  siRNA sequence
cucagaaucccucaaacaa

SEQ ID NO: 309 - RNA, 19 nt, artificial sequence
  siRNA sequence
uuuacaguugcguaguugc

SEQ ID NO: 310 - RNA, 19 nt, artificial sequence
  siRNA sequence
gcaacuacgcaacuguaaa

SEQ ID NO: 311 - RNA, 19 nt, artificial sequence
  siRNA sequence
auauguuucgggaguuuac

SEQ ID NO: 312 - RNA, 19 nt, artificial sequence
  siRNA sequence
guaaacucccgaaacauau

SEQ ID NO: 313 - RNA, 19 nt, artificial sequence
  siRNA sequence
aaaucuaugggcaaugucg

SEQ ID NO: 314 - RNA, 19 nt, artificial sequence
  siRNA sequence
cgacauugcccauagauuu

SEQ ID NO: 315 - RNA, 19 nt, artificial sequence
  siRNA sequence
ugaaaguguucaucaacac

SEQ ID NO: 316 - RNA, 19 nt, artificial sequence
  siRNA sequence
guguugaugaacacuuuca

SEQ ID NO: 317 - RNA, 19 nt, artificial sequence
  siRNA sequence
auguugaucuagaguuucc

SEQ ID NO: 318 - RNA, 19 nt, artificial sequence
  siRNA sequence
ggaaacucuagaucaacau

SEQ ID NO: 319 - RNA, 19 nt, artificial sequence
  siRNA sequence
agaaacaauguguguaugc

SEQ ID NO: 320 - RNA, 19 nt, artificial sequence
  siRNA sequence
gcauacacacauuguuucu

SEQ ID NO: 321 - RNA, 19 nt, artificial sequence
  siRNA sequence
uuagaguuguguugaaucg

SEQ ID NO: 322 - RNA, 19 nt, artificial sequence
  siRNA sequence
cgauucaacacaacucuaa

SEQ ID NO: 323 - RNA, 19 nt, artificial sequence
  siRNA sequence
ucacuauagggcguaaugc

SEQ ID NO: 324 - RNA, 19 nt, artificial sequence
  siRNA sequence
gcauuacgcccuauaguga

SEQ ID NO: 325 - RNA, 19 nt, artificial sequence
  siRNA sequence
auauccuagaacauuaggc

SEQ ID NO: 326 - RNA, 19 nt, artificial sequence
  siRNA sequence
gccuaauguucuaggauau

SEQ ID NO: 327 - RNA, 19 nt, artificial sequence
  siRNA sequence
ucauauugcuaggaaaugc

SEQ ID NO: 328 - RNA, 19 nt, artificial sequence
  siRNA sequence
gcauuuccuagcaauauga

SEQ ID NO: 329 - RNA, 19 nt, artificial sequence
  siRNA sequence
uauugaacccgagaugaug

SEQ ID NO: 330 - RNA, 19 nt, artificial sequence
  siRNA sequence
caucaucucggguucaaua

SEQ ID NO: 331 - RNA, 19 nt, artificial sequence
  siRNA sequence
auauuguuccgaaauccag

SEQ ID NO: 332 - RNA, 19 nt, artificial sequence
  siRNA sequence
cuggauuucggaacaauau

SEQ ID NO: 333 - RNA, 19 nt, artificial sequence
  siRNA sequence
ugauauuguuccgaaaucc

SEQ ID NO: 334 - RNA, 19 nt, artificial sequence
  siRNA sequence
ggauuucggaacaauauca

SEQ ID NO: 335 - RNA, 19 nt, artificial sequence
  siRNA sequence
uuguugaugaucuuagagg

SEQ ID NO: 336 - RNA, 19 nt, artificial sequence
  siRNA sequence
ccucuaagaucaucaacaa

SEQ ID NO: 337 - RNA, 19 nt, artificial sequence
  siRNA sequence
uaucuucuguccuugaucc

SEQ ID NO: 338 - RNA, 19 nt, artificial sequence
  siRNA sequence
ggaucaaggacagaagaua

SEQ ID NO: 339 - RNA, 19 nt, artificial sequence
  siRNA sequence
gccagaaugcuacggagau

SEQ ID NO: 340 - RNA, 19 nt, artificial sequence
  siRNA sequence
aucuccguagcauucuggc

SEQ ID NO: 341 - RNA, 19 nt, artificial sequence
  siRNA sequence
gcugauucagaacaguaua

SEQ ID NO: 342 - RNA, 19 nt, artificial sequence
  siRNA sequence
uauacuguucugaaucagc

SEQ ID NO: 343 - RNA, 19 nt, artificial sequence
  siRNA sequence
gccuuacucaucugaugau

SEQ ID NO: 344 - RNA, 19 nt, artificial sequence
  siRNA sequence
aucaucagaugaguaaggc

SEQ ID NO: 345 - RNA, 19 nt, artificial sequence
  siRNA sequence
ccugaaugaugccacauau

SEQ ID NO: 346 - RNA, 19 nt, artificial sequence
  siRNA sequence
auauguggcaucauucagg

SEQ ID NO: 347 - RNA, 19 nt, artificial sequence
  siRNA sequence
gagaaggguacucccuggu

SEQ ID NO: 348 - RNA, 19 nt, artificial sequence
  siRNA sequence
accagggaguacccuucuc

SEQ ID NO: 349 - RNA, 19 nt, artificial sequence
  siRNA sequence
gcagaggaguaugacaauu

SEQ ID NO: 350 - RNA, 19 nt, artificial sequence
  siRNA sequence
aauugucauacuccucugc

SEQ ID NO: 351 - RNA, 19 nt, artificial sequence
  siRNA sequence
gcacuuacauugaacacaa

SEQ ID NO: 352 - RNA, 19 nt, artificial sequence
  siRNA sequence
uuguguucaauguaagugc

SEQ ID NO: 353 - RNA, 19 nt, artificial sequence
  siRNA sequence
ggauguuucuagcaaugau

SEQ ID NO: 354 - RNA, 19 nt, artificial sequence
  siRNA sequence
aucauugcuagaaacaucc

SEQ ID NO: 355 - RNA, 19 nt, artificial sequence
  siRNA sequence
gcggaaaugcucgcaaaua

SEQ ID NO: 356 - RNA, 19 nt, artificial sequence
  siRNA sequence
uauuugcgagcauuuccgc

SEQ ID NO: 357 - RNA, 19 nt, artificial sequence
  siRNA sequence
gguucuauagaaccugcaa

SEQ ID NO: 358 - RNA, 19 nt, artificial sequence
  siRNA sequence
uugcagguucuauagaacc

SEQ ID NO: 359 - RNA, 19 nt, artificial sequence
  siRNA sequence
ggaguuucaauucugaauc

SEQ ID NO: 360 - RNA, 19 nt, artificial sequence
  siRNA sequence
gauucagaauugaaacucc

SEQ ID NO: 361 - RNA, 19 nt, artificial sequence
  siRNA sequence
gacuccaauccucagauga

SEQ ID NO: 362 - RNA, 19 nt, artificial sequence
  siRNA sequence
ucaucugaggauuggaguc

SEQ ID NO: 363 - RNA, 19 nt, artificial sequence
  siRNA sequence
ggagaagaauccugcccuu

SEQ ID NO: 364 - RNA, 19 nt, artificial sequence
  siRNA sequence
aagggcaggauucuucucc

SEQ ID NO: 365 - RNA, 19 nt, artificial sequence
  siRNA sequence
gguguugcauuugacccaa

SEQ ID NO: 366 - RNA, 19 nt, artificial sequence
  siRNA sequence
uugggucaaaugcaacacc

SEQ ID NO: 367 - RNA, 19 nt, artificial sequence
  siRNA sequence
gcgaagagcaacagccauu

SEQ ID NO: 368 - RNA, 19 nt, artificial sequence
  siRNA sequence
aauggcuguugcucuucgc

SEQ ID NO: 369 - RNA, 19 nt, artificial sequence
  siRNA sequence
gggaaagacgagcaaucaa

SEQ ID NO: 370 - RNA, 19 nt, artificial sequence
  siRNA sequence
uugauugcucgucuuuccc

SEQ ID NO: 371 - RNA, 19 nt, artificial sequence
  siRNA sequence
gcaucaacuccugaggcau

SEQ ID NO: 372 - DNA, 21 bp, artificial sequence
  siRNA sequence
augccucaggaguugaugctt

SEQ ID NO: 373 - RNA, 19 nt, artificial sequence
  siRNA sequence
ggaguagaugaauauucca

SEQ ID NO: 374 - RNA, 19 nt, artificial sequence
  siRNA sequence
uggaauauucaucuacucc

SEQ ID NO: 375 - RNA, 19 nt, artificial sequence
  siRNA sequence
gcucucagcggucgaaauu

SEQ ID NO: 376 - RNA, 19 nt, artificial sequence
  siRNA sequence
aauuucgaccgcugagagc

SEQ ID NO: 377 - RNA, 19 nt, artificial sequence
  siRNA sequence
gcaggugcuagcagaacuu

SEQ ID NO: 378 - RNA, 19 nt, artificial sequence
  siRNA sequence
aaguucugcuagcaccugc

SEQ ID NO: 379 - RNA, 19 nt, artificial sequence
  siRNA sequence
gcagggcuacugaauauau

SEQ ID NO: 380 - RNA, 19 nt, artificial sequence
  siRNA sequence
auauauucaguagcccugc

SEQ ID NO: 381 - RNA, 19 nt, artificial sequence
  siRNA sequence
gcaguaggccaagugucaa

SEQ ID NO: 382 - RNA, 19 nt, artificial sequence
  siRNA sequence
uugacacuuggccuacugc

SEQ ID NO: 383 - RNA, 19 nt, artificial sequence
  siRNA sequence
caacuccuuccucacacau

SEQ ID NO: 384 - RNA, 19 nt, artificial sequence
  siRNA sequence
augugugaggaaggaguug

SEQ ID NO: 385 - RNA, 19 nt, artificial sequence
  siRNA sequence
agggaguguacauaaauac

SEQ ID NO: 386 - RNA, 19 nt, artificial sequence
  siRNA sequence
guauuuauguacacucccu

SEQ ID NO: 387 - RNA, 19 nt, artificial sequence
  siRNA sequence
gcucucauggaguggauaa

SEQ ID NO: 388 - RNA, 19 nt, artificial sequence
  siRNA sequence
uuauccacuccaugagagc

SEQ ID NO: 389 - RNA, 19 nt, artificial sequence
  siRNA sequence
ggacuaguaugugccacuu

SEQ ID NO: 390 - RNA, 19 nt, artificial sequence
  siRNA sequence
aaguggcacauacuagucc

SEQ ID NO: 391 - RNA, 19 nt, artificial sequence
  siRNA sequence
gcacuacggcuaaggcuau

SEQ ID NO: 392 - RNA, 19 nt, artificial sequence
  siRNA sequence
auagccuuagccguagugc

SEQ ID NO: 393 - RNA, 19 nt, artificial sequence
  siRNA sequence
ggaagaauaucggcaggaa

SEQ ID NO: 394 - RNA, 19 nt, artificial sequence
  siRNA sequence
uuccugccgauauucuucc

SEQ ID NO: 395 - RNA, 19 nt, artificial sequence
  siRNA sequence
ggaguggauaaagacaaga

SEQ ID NO: 396 - RNA, 19 nt, artificial sequence
  siRNA sequence
ucuugucuuuauccacacc

SEQ ID NO: 397 - RNA, 19 nt, artificial sequence
  siRNA sequence
cuaacuccaguacaggucu

SEQ ID NO: 398 - RNA, 19 nt, artificial sequence
  siRNA sequence
agaccuguacuggaguuag